(19) United States
(12) Patent Application Publication
(10) Pub. No.: US 2003/0039965 A1
(43) Pub. Date: Feb. 27, 2003

(54) Algorithmic determination of flanking DNA sequences that control the expression of sets of genes in prokaryotic, archea and eukaryotic genomes

(76) Inventor: Richard J. Feldmann, Derwood, MD (US)

Correspondence Address: Law Office of Jim Zegeer, 801 North Pitt Street, #108, Alexandria, VA, 22314, US

(21) Appl. No.: 09/866,925

(22) Filed: May 30, 2001

Related U.S. Application Data
(60) Provisional application No. 60/208,650, filed on Jun. 2, 2000.

(51) Int. Cl. C12Q 1/68

(52) U.S. Cl. 435/6

(57) ABSTRACT

An algorithm has been developed to identify four DNA sequences of 20 bases or more that form a structure called a connectron. Two sequences C1 and C2 are adjacent to each other. These sequences are expressed as RNA in the 3′UTR of some genes in many prokaryotic, archea and eukaryotic genomes. The other half of a connectron is two DNA sequences T1 and T2 that are on the same chromosome and range in distance from each other by about 1 kb to 105 kb. The C1 sequence is identical to the T1 sequence and the C2 sequence is identical to the T2 sequence. C1/C2 and T1-T2 can be on different chromosomes. The C1/C2 RNA sequence of the gene transcript finds the two double-stranded DNA sequences T1 and T2. The single-stranded RNA and double-stranded DNA then form a triple-stranded Hoogsteen helix of the RNA/DNA/DNA variety. Because the C1 sequence is adjacent to the C2 sequence, the T1 sequence is made spatially adjacent to the T2 sequence in a compact X-shaped structure. Chromatin particles form as compact 30 nm assemblies in the DNA between T1 and T2 thus eliminating the intervening genes from promotion and expression. Connectrons remove sets of genes from expression and thus modulate the behavior of many types of cells.

### REFERENCE TO RELATED APPLICATION

The present application is the subject of Provisional Application Ser. No. 60/208,650 filed Jun. 2, 2000 entitled ALGORITHMIC DETERMINATION OF CONNECTRONS FOR THE HIGH LEVEL REGULATION OF GENE EXPRESSION.

### INTRODUCTION

RNA introduced into a cell by a virus is now known to trigger a cellular defense mechanism known as post-transcriptional gene silencing (PTGS). If the viral RNA sequence matches a sequence within the cell's genome the associated genes are turned off or silenced. This phenomenon is also called ‘RNA interference’ or RNAi. A single-stranded RNA can interact with another single-stranded RNA (known as antisense RNA). The single-stranded RNA can also form a triple-stranded complex with double-stranded DNA. This triple-stranded complex is known as a Hoogsteen helix. This patent application shows how two specific adjacent RNA single-stranded sequences (called C1 and C2—for Control Sequence 1 and Control Sequence 2) interact with two distant double-stranded DNA sequences (called T1 and T2—for Target Sequence 1 and Target Sequence 2) to form a tetradic relationship which is called a “connectron”. The two distant DNA double-stranded sequences (T1 and T2) must be on the same chromosome in a genome and they must be between about 1 kb and 105 kb of each other. The adjacent single-stranded RNA sequences (C1/C2) can be on the same or different chromosome as the T1 and T2 sequences. The C1 sequence is identical to the T1 sequence and the C2 sequence is identical to the T2 sequence. The connectron acts to stabilize the double-stranded DNA by allowing 30 nm chromatin particles to form. Genes that lie between the T1 and T2 sequences when wrapped up in 30 nm chromatin particles are not open to promotion and expression. The connectron (i.e. the tetradic relationship between the T1-T2 sequences and C1/C2 sequences) provides a general explanation for PTGS. A connectron can implemented by RNA sequences, PNA (Peptide Nucleic Acid) sequences or by a zinc-finger DNA Binding Protein (DBP) specific to the T1 and T2 sequences.

Characteristically the adjacent C1/C2 sequences lie in the 3′UTR of a gene. The T1 and T2 sequences do not lie within the translated region of any gene. These sequences “surround” one or more genes. There are, however, T1 and T2 sequence pairs that surround one or more C1/C2 sequences that are not 3′UTR to any gene. These are called “geneless connectrons”. There may be promoter sequences that cause the transcription of these 3′UTR sequences.

A computer-based algorithm that is similar to the algorithm used in the U.S. Pat. No. 6,205,404 has been developed to determine the connectron structure of any genome. This algorithm determines the existence of all the connectrons in the genomic DNA. Connectrons exist in prokaryotes, archea, single-celled eukaryotes, multi-celled eukaryotes, plants and higher animals. Connectron relationships exist between prokaryotes and their plasmids. The geneless connectrons provide a possible mechanism for forming a hierarchy of gene expression control that will produce an understanding of cell differentiation and tissue development.

Each connectron is a unique tetrad of sequences. Each connectron changes the expression of the genes between the T1 and T2 sequences. The C1 sequence (which is equivalent to the T1 sequence) and the C2 sequence (which is equivalent to the T2 sequence) are determined by the invention described in this patent application. In general, the tetrad of connectron sequences can be patented because the structure of matter is known and the function of specific gene expression modulation is also known. Gene expression modification can be produced by introducing antisense RNA or PNA to interact C1/C2 RNA sequences or zinc-finger DBPs to interact with the T1 and T2 sequences. Using connectrons it will be possible to modify cellular and tissue behavior in a very general manner.

Examples will be given from different genomes to illustrate that the connectron is a perfectly general and universal concept.

Definitions

Double stranded DNA—Watson and Crick showed in 1953 that DNA naturally forms a double-stranded helix. A typical double stranded sequence is

5′-TAGAGGAGTACCAC-3′
3′-ATCTCCTCATGGTG-5′

Hydrogen Bond—The force between a hydrogen atom and another heavier atom such as Oxygen (O), Nitrogen (N), Phosphorus (P), or Sulfur (S).

Positive strand—The positive strand is normally represented 5′ to 3′ running left to right as in

5′-TAGAGGAGTACCAC-3′

Negative strand—The negative strand is normally represented 5′ to 3′ running right to left as in

3′-ATCTCCTCATGGTG-5′

Single stranded RNA—Either the positive or the negative strand of the double-stranded DNA can be transcribed by the polymerase. In RNA U replaces T.

| | |
|---|---|
| RNA of positive strand sequence | 5′-UAGAGGAGUACCAC-3′ |
| RNA of negative strand sequence | 5′-GUGGUACUCCUCUA-3′ |

Antisense RNA—The antisense strand of any RNA sequence is the compliment sequence

| | |
|---|---|
| RNA sequence | 5′-UAGAGGAGUACCAC-3′ |
| Antisense RNA sequence | 3′-AUCUCCUCAUGGUG-5′ |

Triple Strand Helix—The RNA sequence of a RNA/DNA triple-strand complex is the same as the positive strand of the DNA

| | |
|---|---|
| DNA positive strand | 5′-TAGAGGAGTACCAC-3′ |
| DNA negative strand | 3′-ATCTCCTCATGGTG-5′ |
| RNA strand | 5′-UAGAGGAGUACCAC-3′ |

Promoter—Any region of DNA, that binds proteins which engage the polymerase transcription mechanism.

TATA Box—A region near the 3′ end of a promoter with the sequence TATA.

mRNA—The RNA produced from the DNA by the polymerase as a result of transcription

Start of transcription—The 3′ end of a promoter where the polymerase mechanism begins to transcribe DNA into mRNA.

Exon—Any region of mRNA which is used to code for proteins

Intron—Any region of mRNA lying between two exons which is not used to code for proteins. The introns are edited out of the initial RNA transcript to form the mature mRNA.

3′ UTR—The untranslated 3′ end of an mRNA is beyond the end of the last exon. A stop codon in the mRNA causes the ribosome to stop the translation of mRNA into protein.

End of translation—The 3′ end of the 3′-most exon.

Translated region—Any collection of exons and introns.

Gene—Any DNA region that codes for a protein. Introns do not occur in prokaryotic genes and they sometime fail to occur in eukaryotic genes. A typical model of a gene is

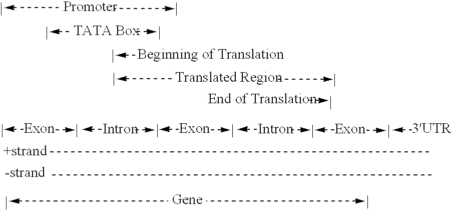

Positive strand gene—Any gene in which the features run 5′ to 3′ on the positive strand

Negative strand gene—Any gene in which the features run 5′ to 3′ on the negative strand

C1 sequence—Any positive or negative strand DNA sequence of 20 bases or more.

The C2 sequence must occur in the same chromosome as the C1 sequence.

C2 sequence—Any positive or negative strand DNA sequence of 20 bases or more. The C1 sequence must occur in the same chromosome as the C2 sequence.

C1/C2—Any positive or negative strand DNA sequence of 40 or more bases such that the C1 sequence is adjacent to the C2 sequence

T1 sequence—Any positive or negative strand DNA sequence of 20 bases or more that is on the same chromosome as the T2 sequence. The T1 and T2 sequences must be between about 1 kb and 105 kb apart.

T2 sequence—Any positive or negative strand DNA sequence of 20 bases or more that is on the same chromosome as the T1 sequence. The T2 and T1 sequences must be between about 1 kb and 105 kb apart.

Last exon gap or Gap-Distance—The number of bases between the end of transcription and the beginning of the C1/C2 sequence. In prokaryotes and single-celled eukaryotes this gap can range from no bases to 500 bases. In multi-celled eukaryotes the gap can be as large as 10,000 bases.

Poly-adenylation signal—A number of Adenosine (A) bases are added to the mRNA at the end of the 3′UTR.

Possible Connectron—Any set of T1, T2 and C1/C2 sequences such that the C1 sequence is identical to the T1 sequence and the C2 sequence is identical to the T2 sequence. The promoter of some gene causes the mRNA of the gene to be expressed. The mRNA is edited to eliminate the introns. The whole mRNA including the 3′UTR can move about in the cell or the nucleus of the cell. The C1/C2 RNA that is part of the 3′UTR moves to the T1 and T2 DNA sequences. A triple-stranded complex of the DNA and the RNA forms such that the C1 sequence forms hydrogen bonds with the T1 sequence and the C2 sequence forms hydrogen bonds with the T2 sequence.

Because the C1 sequence is adjacent to the C2 sequence, the T1 sequence is brought physically close to the T2 sequence. This produces a loop of between about 1 kb and 105 kb in the DNA. Histone proteins reduce the length of the DNA by binding 200 bases. Histone/DNA complexes form six-fold symmetry chromatin assemblies. The diameter of the chromatin assemblies is approximately 30 nm.

Real Connectron—Any Possible Connectron which is within the Gap-Distance of some gene

Homologous connectron—The T1 sequence and the T2 sequence are on the same chromosome as the C1/C2 sequence

Heterologous connectron—The T1 sequence and the T2 sequence are on a chromosome different from chromosome of the C1/C2 sequence

Permanent connectron—Any C1/C2 sequence, which is 3′ UTR to some gene that is not surrounded by any T1 and T2 sequence pairs

Transient connectron—Any C1/C2 sequence, which is 3′ UTR to some gene that is surrounded by one or more T1 and T2 sequence pairs

Self-limiting connectron—Any C1/C2 sequence which is 3′UTR to some gene that is surrounded by the T1 and T2 sequences such that C1=T1 and C2=T2

Geneless connectron—Any C1/C2 sequence which is not 3′UTR to some gene but is surrounded by some T1 and T2. A promoter may lie 5′ to the C1/C2 sequence.

Bidirectionality of Connectron Excitation—A C1/C2 short loop on one strand selects a T1-T2 long loop pair on the same or the opposite strand. The C1/C2 short loop has a complementary C1′/C2′ sequence on the opposite strand. Similarly the T1-T2 long loop pair has a complementary long loop pair T1′-T2′. Wherever a C1/C2, T1-T2 tetrad exists there is a complementary C1′/C2′, T1′-T2′ tetrad. The C1/C2 short loop can be transcribed as a 3′UTR to a gene on the same strand. The C1′/C2′ short loop which is on the strand opposite to the C1/C2 short loop can also can be transcribed as a 3′UTR to a gene on the same strand. There are four possible models of action

```
                      T1 T2 gene− C1/C2
+ strand ---------------------------------------------
− strand ---------------------------------------------
                      T1 T2
+ strand ---------------------------------------------
− strand ---------------------------------------------
                                                                      C2/C1 − gene
+ strand ---------------------------------------------
− strand ---------------------------------------------
                            T2′ T1′ C2′/C1′− gene
                                                    gene − C1′/C2′
+ strand ---------------------------------------------
− strand ---------------------------------------------
                T2′ T1′
```

Of course, the short loops and the long loops do not have to be on the same chromosome.

Hierarchy of connectron action—When a C1/C2 is expressed it forms a T1-T2 loop by forming a connectron. The C1/C2 sequence does not have to be on the same chromosome as the T1 and T2 sequences. This provides a way of causing interaction between chromosomes. When the T1-T2 loop forms, any genes in that loop region which had been expressing C1/C2 sequences in their 3′UTRs, now cease expressing the C1/C2 sequences. The connectrons formed by these C1/C2 sequences will cease to exist after some time thus opening up the genes inside the respective T1-T2 loops to expression. The hierarchy of connectron action is alternates between repression and expression. The connectron hierarchies can be of any depth.

One-to-Many connectron action—One C1/C2 sequence can form connectrons in many different places on many different chromosomes. The only requirement is that C1=T1 and C2=T2. This makes it possible for one expression event to control the expression of many genes on different chromosomes.

Many-to-One connectron action—C1/C2s that come from many different places on many different chromosomes can form a connectron for a specific T1-T2 sequence pair. The only requirement is that C1=T1 and C2=T2. This makes it possible for many different expression events to control the expression of one set of genes on a particular chromosome.

Many-to-Many connectron action—The arrangement of C1/C2s and T1-T2s across chromosomes can form a complex web of gene expression control relationships.

Percentage of the Genome Regulated by Connectrons—Since the connectrons for a sequenced genome can be calculated, the percentage of the genome that is open to connectron regulation can be known.

Emergent Property—The network of connectrons in any genome emerges from a knowledge of the complete DNA sequence of the genome. Because both the C1/C2 sequences and the T1-T2 sequences can be any place in the genome, the whole genomic sequence must be known before all the connectrons can be determined.

Paradigm Shift—For the past fifty years since the discovery by Watson and Crick of the double-helical nature of DNA, the reigning paradigm for scientific discovery has been the study of one gene and its effects on the behavior of a cell. The advent of genomic sequencing and this invention of connectrons that emerge from the whole genome will produce a shift in the way scientists view biological systems and the way they formulate and execute experiments. The many-to-many relationships between the connectrons means that there are many ways in which the expression of a set of genes can be modulated. The multiplicity of control pathways means produces a system stability that makes it possible for biological systems to be stable for long periods of evolutionary time. The thinking that goes into formulating scientific experiments will have to change to accommodate the changes in understanding that will be induced by the application and extension of this patent application.

Hierarchy of DNA Structuring—The DNA of a cell's genome is structured in a hierarchy of six levels. FIGS. 1, 2 and 3 have been adapted from The Molecular Biology of the Cell by Alberts, Bray, Lewis, Raff, Roberts and Watson [third edition pages 354, 345 and 348]. As shown in FIG. 1, the double stranded DNA is level 1. The double-stranded DNA is wrapped around histone proteins to form a chromatin particle that is level 2 of the hierarchy. Level 2 is described as “beads-on-a-string” in FIG. 1. The chromatin particles are packed in a six-fold symmetry as shown in FIG. 2a and FIG. 2b. These six-fold assemblies have a diameter of 30 nm. Each 30 nm assembly contains from 18 (i.e. 6*3) to 30 (i.e. 6*5) chromatin particles. The 30 nm assemblies aggregate into large loops which range in length from 5,000 bases to 100,000 bases of DNA. The size of these large loops as shown in FIG. 1 is approximately 300 nm. These large loops constitute level 4 of the structuring hierarchy. As shown in FIG. 1, level 5 of the DNA structuring hierarchy many large loops are condensed to form a structure which is approximately 700 nm in diameter. The complete chromosome that constitutes level 6 of the hierarchy is composed of two very long sections of level 5 DNA.

Model of Chromatin Structure—The level 4 structure of DNA as shown in FIG. 1 ranges in length from 5,000 to 105,000 bases of DNA. FIG. 3 shows that proteins are thought to connect portions of the long loops formed by the 30 nm particles to form a chromosome axis. These condensed long loops are described as chromomeres in The Molecular Biology of the Cell.

### PRIOR ART

The chromomere model of DNA structuring was presented by N. A Resnik, et al.[1] and is based on electron microscopic data. There are more recent papers studying a variety of genomes with electron microscopy but no equivalent study of chromomeres has been done on a fully sequenced genome.

A recent News Feature in Nature by T. Gura [2] described the discovery of post-transcriptional gene silencing in which viral RNA interacts with the transcribed RNA of the cell to silence the expression of genes. This article describes experiments in C. elegans and D. megalomaster in which RNA that is complementary to mRNA introduced into a cell. This “antisense” RNA has the effect of turning off the expression of one or more genes. The introduced complementary RNA produces an “RNA interference” called RNAi.

Thomas Werner and his colleagues at Genomatix in Munich, Germany have developed an approach to understanding what they call “Matrix Attachment Region” (MAR). FIG. 5 shows their interpretation of the structure of DNA surrounding a gene. The following description of the MAR is copied from the Genomatrix web site

“Matrix Attachment Regions (MARs) MARs are sequence regions that are responsible for the attachment of genomic DNA to the nuclear matrix or scaffold. Transcription absolutely requires anchorage of genomic DNA to the nuclear matrix.

Functional features of MARs:

Anchoring of regulatory elements like promoters and enhancers to the nuclear matrix.

Ensuring long term activity of promoters and enhancers in chromatin.

Insulation, rendering a functional domain insensitive to position effects.

Genomatix is conducting a research project to define and detect MARs by computer-analysis.”

### BRIEF DESCRIPTION OF THE OBJECTS OF THE INVENTION

An object of the invention is to provide a method of identifying DNA sequences that control the expression of different collections of genes in a genome comprising, detecting selected DNA sequences adjacent to some genes excluding exons and introns.

An object of the invention is to provide a method of identifying DNA sequences that control the expression of different collections of genes comprising, detecting, by computer, one or more pairs of non-adjacent DNA sequences to which are bound to two RNA sequences.

An object of the invention is to provide a method of identifying DNA sequences that control the expression of different collections of genes in a genome comprising detecting changes in connectron behavior in the genome.

An object of the invention is to provide a method of modifying the expression of different gene collections in a genome, comprising detecting changes in connectron behavior as a result of an exogenous stimulus.

An object of the invention is to provide a method of detecting where and when new genes are being integrated into a host genome comprising detecting the connectrons in said host genome.

An object of the invention is to provide a method of detecting the expression effect of different gene collections in a given body comprising detecting the back and forth flow of connectrons between the chromosomes thereof.

An object of the invention is to provide a method of modifying a given body comprising modifying the connectron organization therein.

An object of the invention is to provide a method of detecting connectron control and target sequences in a given genome comprising:

determining the base composition of said genome,

determining one or more sites of control sequence organization, and/or

determining one or more sites of target application.

An object of the invention is to provide a method of determining the response of a cell in any tissue to changes in the cell's environment and/or genetic composition comprising providing a complete genomic DNA sequence for the organism and determining the effect of changes in connectrons due to application of a given exogenous stimulus to the gnome.

An object of the invention is to provide a method of determining in prokaryotes, archea, single-celled eukaryotes and multi-celled eukaryotes, the tetradic relationship T1=C1 and T2=C2 where T1 and T2 are DNA sequences 20 or more bases in length, where the C1 sequence is adjacent to the C2 sequence, where the T1 and T2 sequences are on the same chromosome, and where the C1/C2 sequences are on the same chromosome as T1 and T2 or where the C1/C2 sequences are on a chromosome different from T1 and T2, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 40 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart, and

T2 sequence—any positive or negative strand DNA sequence of 20 bases or more that is on the same chromosome as the T1 sequence, the T2 or T1 sequences must be between about 1 kb and 105 kb apart.

An object of the invention is to provide a method of determining in prokaryotes, archea, single-celled eukaryotes and multi-celled eukaryotes, the connectron relationship that permits many different C1/C2 short loops to control the existence of a T1-T2 long loop and wherein said C1/C2 short lops can be on the same chromosome or on different chromosomes from the T1-T2 long loop, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 40 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart, and

T2 sequence—any positive or negative strand DNA sequence of 20 bases or more that is on the same chromosome as the T1 sequence, the T2 or T1 sequences must be between about 1 kb and 105 kb apart.

An object of the invention is to provide a method of determining in prokaryotes, archea, single-celled eukaryotes and multi-celled eukaryotes, the connectron relationship that permits one C1/C2 short loop to control the existence of many T1-T2 long loops, the C1/C2 short loop can be on the same chromosome or on different chromosomes from the T1-T2 long loops, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 40 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart, and

T2 sequence—any positive or negative strand DNA sequence of 20 bases or more that is on the same chromosome as the T1 sequence, the T2 or T1 sequences must be between about 1 kb and 105 kb apart.

An object of the invention is to provide a method of determining in the connectron relationships between prokaryotes and their plasmids wherein said connectrons implement a control mechanism between the two genomes that makes it possible from them to form a symbiotic relationship, and in the case of D. radiodurans the relationship is not symmetric, and the D. radiodurans genome sends C1/C2 short loops to the MP1 plasmid, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 40 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart, and

T2 sequence—any positive or negative strand DNA sequence of 20 bases or more that is on the same chromosome as the T1 sequence, the T2 or T1 sequences must be between about 1 kb and 105 kb apart.

An object of the invention is to provide a method of determining that connectron relationships that exist in plant and higher animals.

An object of the invention is to provide a method of determining in prokaryotes, archea, single-celled eukaryotes and multi-celled eukaryotes, the connectron relationship that permits one C1/C2 short loop to control the existence of one or more T1-T2 long loops without being subject to any expression controls other than those of the gene to which the C1/C2 is 3′UTR, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 540 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart,

T2 sequence—any positive or negative strand DNA sequence of 20 bases or more that is on the same chromosome as the T1 sequence, the T2 or T1 sequences must be between about 1 kb and 105 kb apart, and

3′UTR—untranslated 3′ end of an mRNA is beyond the end of the last exon, a stop codon in the mRNA causes the ribosome to stop the translation of mRNA into protein.

An object of the invention is to provide a method of determining in prokaryotes, archea, single-celled eukaryotes and multi-celled eukaryotes, the connectron relationship that permits one C1/C2 short loop to control the existence of one or more T1-T2 long loops such that this C1/C2 short loop is itself subject to expression control by another T1-T2 long loop which surrounds it, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 540 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart, and

T2 sequence—any positive or negative strand DNA sequence of 20 bases or more that is on the same chromosome as the T1 sequence, the T2 or T1 sequences must be between about 1 kb and 105 kb apart.

An object of the invention is to provide a method of determining in prokaryotes, archea, single-celled eukaryotes and multi-celled eukaryotes, the connectron relationship that permits one C1/C2 short loop to control the existence of the T1-T2 long loop that surrounds it, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 40 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart, and

T2 sequence—any positive or negative strand DNA sequence of 20 bases or more that is on the same chromosome as the T1 sequence, the T2 or T1 sequences must be between about 1 kb and 105 kb apart.

An object of the invention is to provide a method of determining the connectron relationships that do not have any genes within the T1-T2 long loop, wherein:

T1 sequence is any positive or negative strand DNA sequence of 20 bases or more that is on the same chromosome as the T2 sequence, and

T2 sequence—any positive or negative strand DNA sequence of 20 bases or more that is on the same chromosome as the T1 sequence, and the T2 or T1 sequences must be between about 1 kb and 105 kb apart.

An object of the invention is to provide a method of determining the geneless connectron relationship where one C1/C2 short loop controls the existence of many geneless T1-T2 long loops, wherein:

C1 sequence—any positive or negative strand DNA sequence of 20 bases or more, the C2 sequence must occur in the same chromosome as the C1 sequence,

C2 sequence—any positive or negative strand DNA sequence of 20 bases or more, the C1 sequence must occur in the same chromosome as the C2 sequence,

C1/C2—any positive or negative strand DNA sequence of 40 or more bases such that the C1 sequence is adjacent to the C2 sequence,

T1 sequence—any positive or negative strand DNA sequence of 20 bases or more that is on the same chromosome as the T2 sequence, the T1 and T2 sequences must be between about 1 kb and 105 kb apart, and

T2 sequence—any positive or negative strand DNA sequence of 20 bases or more that is on the same chromosome as the T1 sequence, the T2 or T1 sequences must be between about 1 kb and 105 kb apart.

### DESCRIPTION OF THE DRAWINGS AND TABLES

The above and other objects, advantages and features of the invention will become more apparent when considered with the following specification and accompanying drawings and tables wherein:

FIG. 1 DNA is structured in six levels of increasing condensation. Double stranded DNA is level 1. Two turns of DNA are wrapped about each chromatin particle at level 2. The chromatin particles which each containing 200 base pairs form into 30 nm particles at level 3. The 30 nm particles form into large loops with an approximate dimension of 300 nm at level 4. Metaphase chromosomes form a condensed structure with an approximate dimension of 700 nm at level 5. An entire metaphase chromosome has a width of approximately 1400 nm at level 6. The large loops at level 4 of the DNA structuring are thought to have between 20,000 (20 kb) and 100,000 (100 kb) base pairs.

The Molecular Biology of the Cell by Alberts, Bray, Lewis, Raff, Roberts and Watson, 3rd. ed., Garland Publishing, Inc., New York, 1994, p. 354

FIG. 2 (a) Chromatin DNA forms into a six-fold symmetry 30 nm particles.

(b) The six-fold symmetry 30 nm particles form a linear chain with a varying number of repeat units.

The Molecular Biology of the Cell by Alberts, Bray, Lewis, Raff, Roberts and Watson, 3rd. ed., Garland Publishing, Inc., New York, 1994, p. 345

FIG. 3 Long loops of 30 nm particles are thought to be closed at the bottom of the loop by proteins.

The Molecular Biology of the Cell by Alberts, Bray, Lewis, Raff, Roberts and Watson, 3rd. ed., Garland Publishing, Inc., New York, 1994, p. 348

FIG. 4 (a) Transcription and Editing. (b) Movement of the RNA through the Nucleus. (c) Connectron Formation

FIG. 5 Overview of schematic organization of a typical transcriptionally active chromosomal loop.

From http://genomatix.gsf.de/funcgenomics/functional_genomics.html

Table 1 Connectron Properties for Prokaryotic, Archea and Eukaryotic Genomes

Table 2 Yeast Inter-Chromosomal Connectron Distribution

FIG. 6 Genome size plotted as a log-log function of the Number of Connectrons

FIG. 7 Number of Sequence Instances plotted as a function of the Number of Fragments

FIG. 8 Level 0—The overall view of the algorithm

FIG. 9 Level 1—Process Flow of the Algorithm

FIG. 10 Level 2a—two pages—Process Genome into Blocking Fragment File

FIG. 11 Level 2b—two pages—Compute the Connectrons for a Genome

FIG. 12 Level 2c—two pages—Analyze Possible Connectrons

FIG. 13 Level 3a—Setup Genome Usage Memory

FIG. 14 Level 3b—Find DBP-Size Blocking File for T1-Window

FIG. 15 Level 1—Find DBP-Size Blocking File for T2-Window

FIG. 16 Level 2a—two pages—Find C1/C2 Entries

FIG. 17 Level 2b—two pages—Scan Genome Usage Memory for Potential Connectrons

### DESCRIPTION OF THE INVENTION

A connectron is a relationship among four DNA sequences. Each sequence must be at least 20 bases long. There is a report by Sharp and Zamore [3] that RNA sequences of “about length 25” are important as sources of RNAi. 27 bases were actually used as the minimum length of each of the sequences. The T1 sequence is on one strand of some chromosome in a genome. The T2 sequence is on the same strand of the same chromosome as the T1 sequence. The T1 and T2 sequences (which are each at least 20 bases in length) must be at least 5,000 bases distant from each other but they can not be more than 105,000 bases distant from each other. The C1 sequence and the C2 sequence (which are each at least 20 bases in length) are adjacent to each other on some strand of some chromosome in the genome. The C1/C2 sequences—called the “short loop”—can be on the same strand as the T1 and T2 sequences or they can be on the opposite strand. The C1/C2 sequences of the short loop can be on the same chromosome as the T1 and T2 sequences but they can also be on a different chromosome in the genome. When a genome has only one chromosome, then the point is moot. Many genomes, of course, have several chromosomes. The C1 sequence is identical to the T1 sequence and the C2 sequence is identical to the T2 sequence.

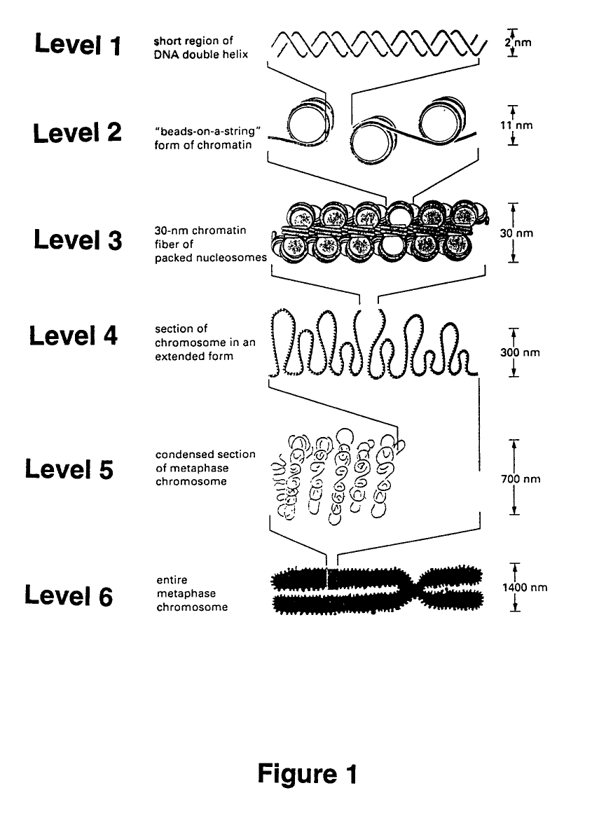

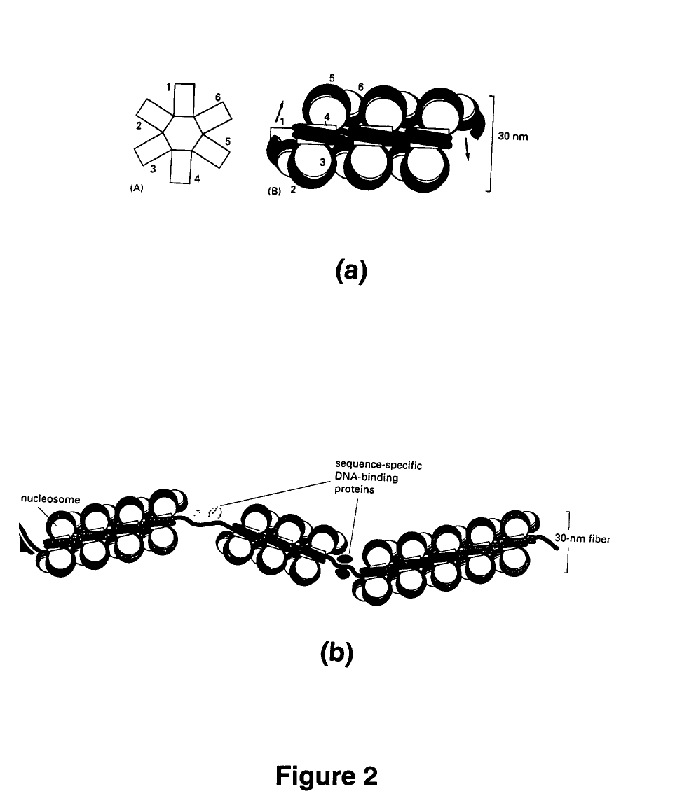

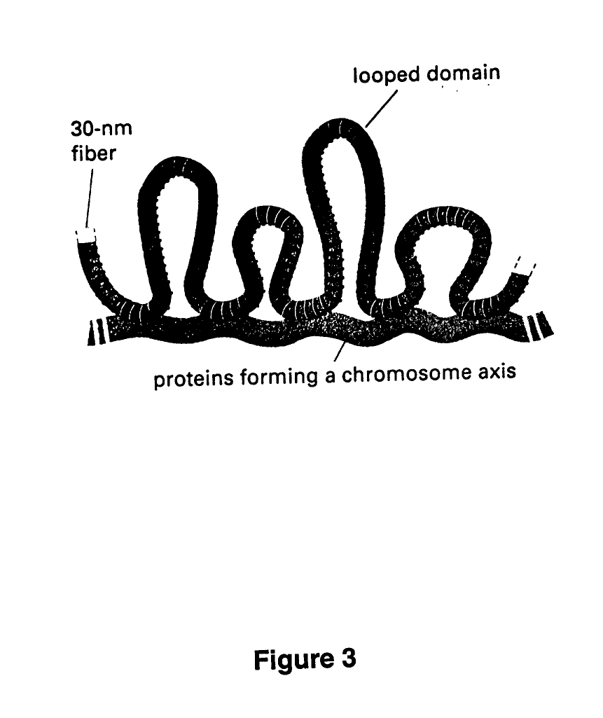

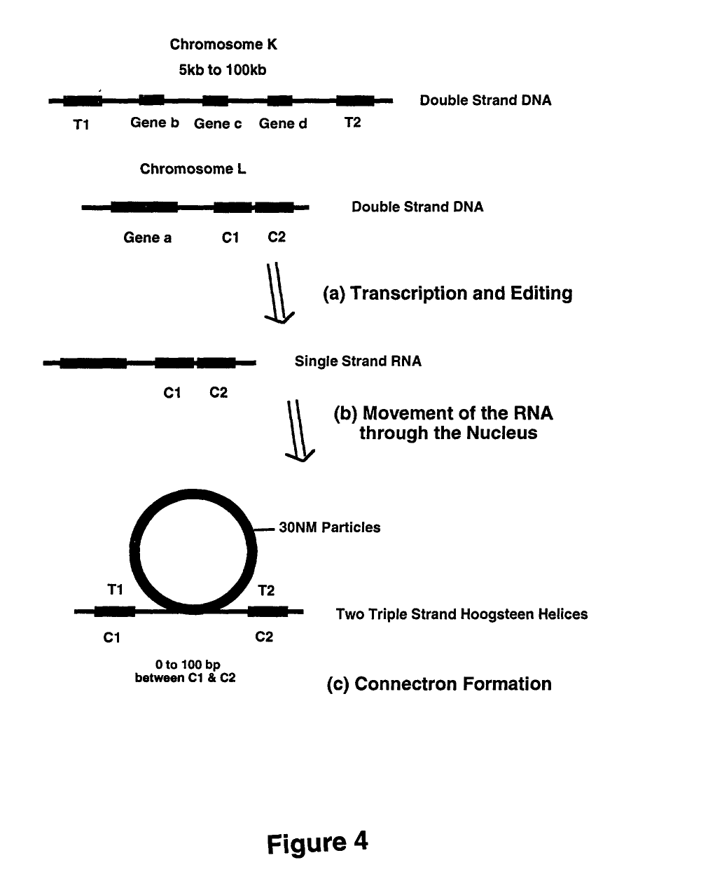

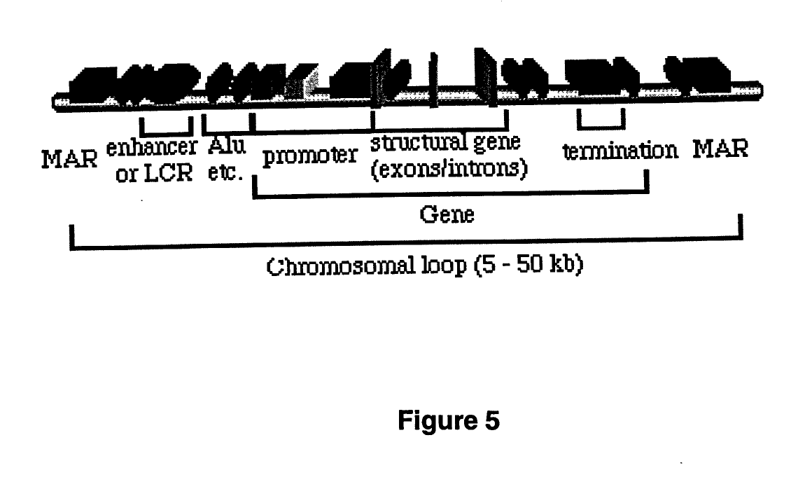

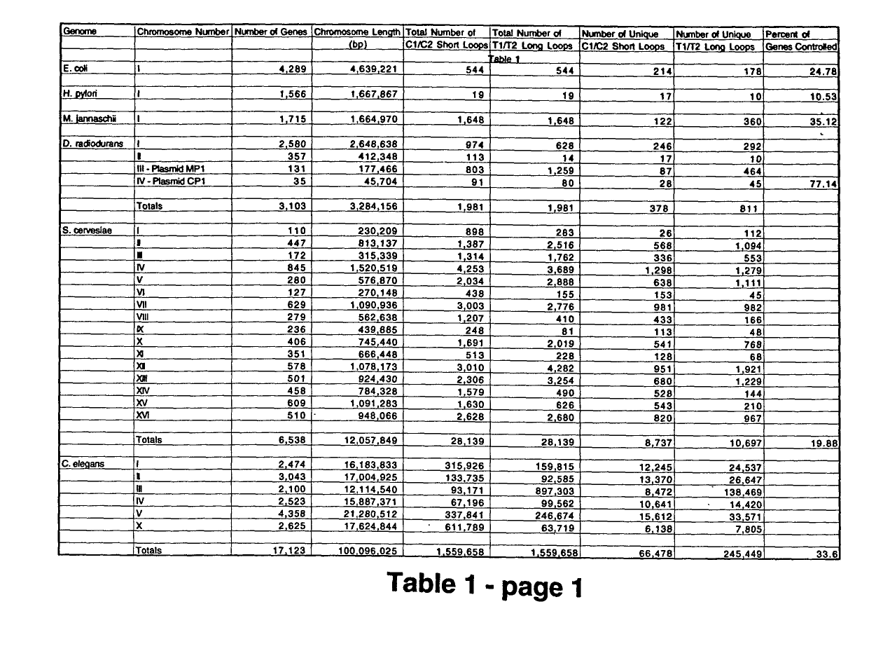

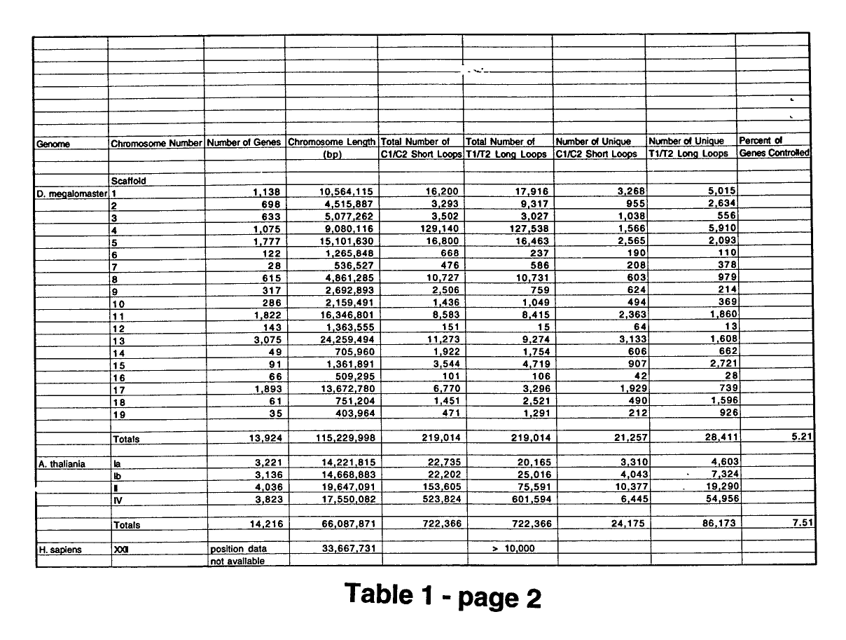

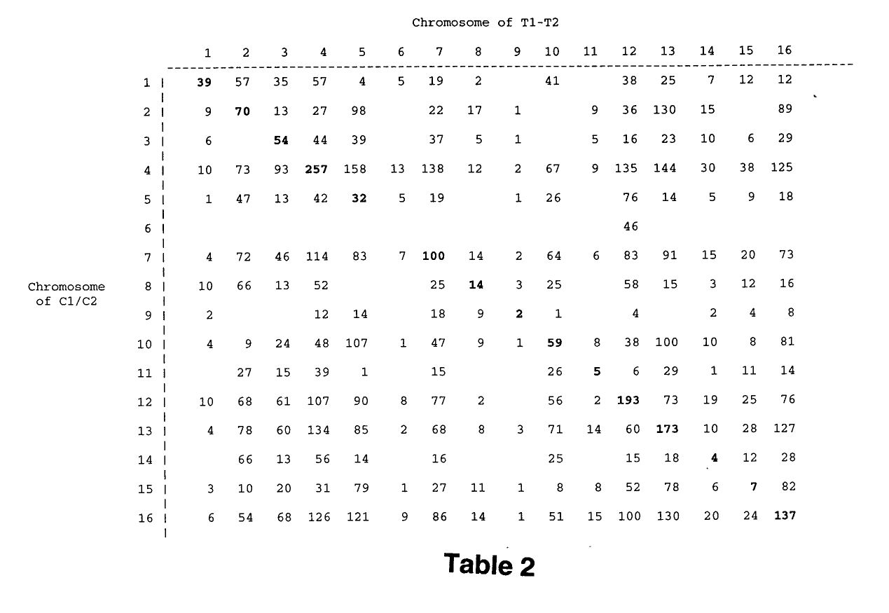

The C1/C2 sequence must be on the same strand as a gene, either be directly adjacent to the gene (i.e. a gap of 0 bases) for prokaryotic genomes or at this time be within 10,000 bases for eukaryotic genomes. The size of the gap between the end of the gene and the beginning of the C1/C2 sequence is a variable. The C1/C2 short loop is expressed as the 3′UTR (Un-Translated Region) of the gene. In the case of prokaryotic genes that do not normally have introns, the whole mRNA becomes the active species for connectron formation. In the case of eukaryotic genes, the whole transcript is the active species for connectron formation upon editing of the transcript to eliminate the introns. The whole transcript then can move about in the cytoplasm of prokaryotic cells or the nucleus of eukaryotic cells. Since the C1 sequence is equivalent to the T1 sequence and the C2 sequence is equivalent to the T2 sequence, the C1 RNA can form a Hoogsteen triple-stranded RNA/DNA/DNA helix with the double-stranded T1 sequence. Similarly the C2 RNA can form a Hoogsteen triple-stranded RNA/DNA/DNA helix with the double-stranded T2 sequence. Because the C1 sequence and the C2 sequence are adjacent to each other, the C1/T2 RNA/DNA/DNA Hoogsteen triple helix is brought into physical adjacency to the C2/T2 RNA/DNA/DNA Hoogsteen triple helix. RNA/DNA/DNA hybrid helices are the most stable form of triple helix. RNA double helices, DNA double helices, RNA triple helices and DNA triple helices are all significantly less stable than a RNA/double-stranded DNA triple helix. The stable physical adjacency of the two triple-stranded Hoogsteen helices ensures that the long loop of double-stranded DNA between the T1 sequence and the T2 sequence can then be structured into 30 nm chromatin particles as shown in level 4 of FIG. 1. The genes on either strand of the DNA between the T1 sequence and the T2 sequence when they are structured into the 30 nm chromatin particles are not open to promotion and expression.

The tetradic relationship between the T1 and T2 sequences that form the long loop and the C1/C2 sequences that form the short loop are called a connectron. The name “connectron” was suggested by J. David Rawn Ph.D. of Towson University. A connectron is possible if the T1, T2, C1 and C2 sequences exist. A connectron is real if the C1/C2 short loop sequence is adjacent to an expressible gene. If the expression of the adjacent gene is inside one or more T1 —T2 long loops then this connectron is said to be transient. If the adjacent gene is not inside any possible T1-T2 long loop then the connectron is said to be permanent. If a connectron is inside of a T1-T2 long loop that has the same sequences (i.e. T1 is really equal to C1 and T2 is really equal to C2) then the connectron is said to be self-limiting. This is true because once the C1/C2 sequence is expressed it forms the T1-T2 long loop that then shuts off the expression of the gene adjacent to the C1/C2 sequence. Self-limiting conectrons can also be called “spike” connectrons since they generate a short-duration spike of the C1/C2 short loop sequence. If a T1-T2 long loop does not contain any genes but it contains C1/C2 short loop sequences then this type of connectrons is said to be geneless. The C1/C2 short loops within a geneless T1-T2 long loop can, of course, control the expression of genes.

The physical existence and lifetimes of the connectrons must be proved by molecular biological experimentation. This physical experimental process, however, is logically quite separate from the computational experimentation that have been conducted from June of 1999 to May of 2001. The computational search for the existence of connectrons has been extremely positive. These computations have shown that connectrons exist in prokaryotes, in archea, between prokaryotes and their plasmids, in single-celled eukaryotes, in multi-celled eukaryotes, in plants, in higher animals and in humans. All of these features and properties are described in the claims section that follows.

The connectron invention is very powerful. It depends only on sequence equivalency. The minimum length of the four sequences seems to be about 20 bases. In the calculations shown in this patent application, 27 bases have been used as a minimum. The Nature News Feature [1] says that other scientists have found RNA sequences of length about 25 that have interesting gene silencing properties. The Nature article does not give any mechanism. Because of my algorithm and its use on a variety of genomes, this patent application provides the computational proof that a particular mechanism is highly probable. The connectron invention provides an explanation for how communication occurs with a chromosome as well as between chromosomes in genomes that have more than one chromosome. Since each T1-T2 long loop can contain one or more genes, the connectron invention provides a mechanism for turning on and turning off sets of genes simultaneously. In time, the connectron invention will provide an explanation for how differentiation of how one cell's behavior differs from the behavior of another adjacent cell. It is already clear from the computational experiments that have been made on S. cervesiae, C. elegans and D. megalomaster that the number of geneless connectrons increases dramatically as evolution proceeds from single-celled eukaryotes (i.e. S. cervesiae) to 1,000 cell eukaryotes (i.e. C. elegans) to visible creatures (i.e. D. megalomaster). The extension of this evolutionary progress to plants (i.e. A. thaliania) for which only three chromosomes are sequenced and humans (i.e. H. sapiens) for which only one chromosome is completely sequenced. Although the complete human genome was published in Nature and Science in February of 2001, the NIH-sponsored genomic sequencing results are available for about ⅓ of the bases in the whole genome. The human genomic sequence determined by Celera Genomics, Inc. is available only by subscription. Table 1 shows how the genome size, the number of genes, the number of gene-containing and geneless connectrons and the percentage of genes controlled are related in many different genomes.

The C1/C2 short loops originate on one chromosome. The T1-T2 long loops can be on the same or different chromosomes. Table 2 which is for yeast (S. cervesiae) is a square matrix of how many C1/C2 short loops on a given chromosome are sent to form T1-T2 long loops on other chromosomes. The diagonal of this matrix shows that many chromosomes send connectrons to themselves. The striking feature of this particular table is that chromosome 6 only sends connectrons to chromosome 12 but that it receives connectrons from chromosomes 4,5,7,10,12,13,15 and 16.

Any tetrad of connectron sequences (i.e. the T1, T2, C1 and C2 sequences) as well as the fact of the adjacency of the C1/C2 short loop sequence to the transcribing gene can be patented because the content of matter and the utility can be exactly described. The utility of a connectron is that the T1-T2 long loop shuts off the expression of the genes that lie between the T1 sequence and the T2 sequence. In the case of geneless connectrons, the utility is of a higher level in that the C1/C2 short loops contained in the higher-level geneless T1-T2 long loop, eventually form other lower-level T1-T2 long loops around a set of genes.

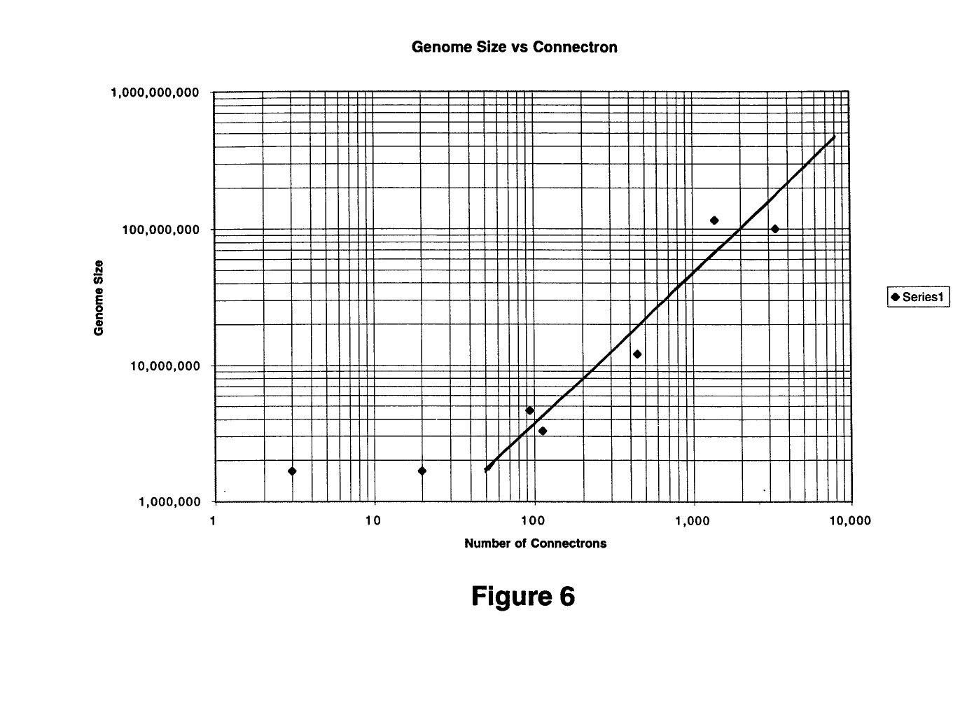

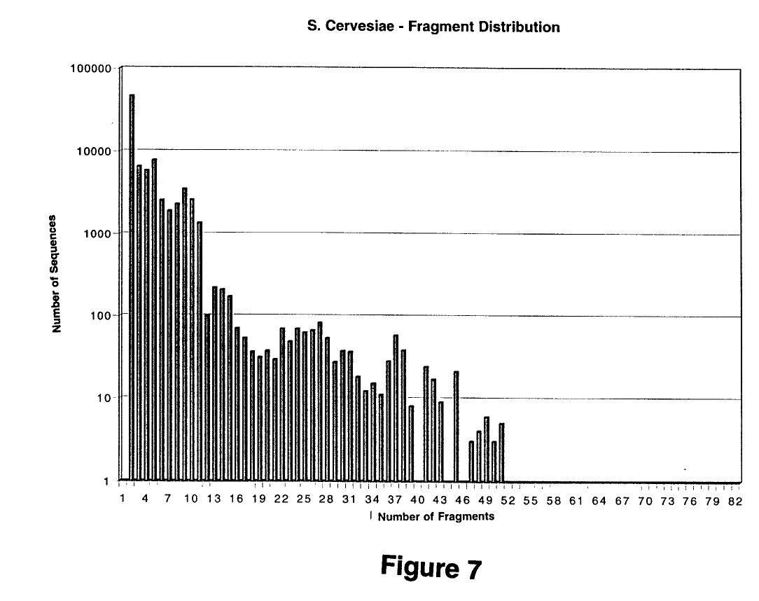

The invention of connectrons comes at a particularly important time in biological discovery. The geneless connectrons make a many-to-many hierarchical control mechanism possible. It is already clear from the determination of the conectrons for C. elegans and D. megalomaster that there are as many or more geneless connectrons than there are genes. It has been clear for some time that the number of genes in a genome is not particularly correlated with the size of the genome. FIG. 6 shows that the size of a genome is roughly linearly correlated with the number of connectrons.

The connectron invention can be used to generate a model of behavior in any cell. The simulation of connectron behavior in different genomes will be the subject of another patent application.

The connectron invention provides for a rational exploitation of the information contained in the raw genomic DNA sequence by forming a hierarchy of relationships between geneless connectrons, transient connectrons, permanent connectrons, self-limiting connectrons and the expression of genes.

### DETAILED DESCRIPTION OF THE INVENTION

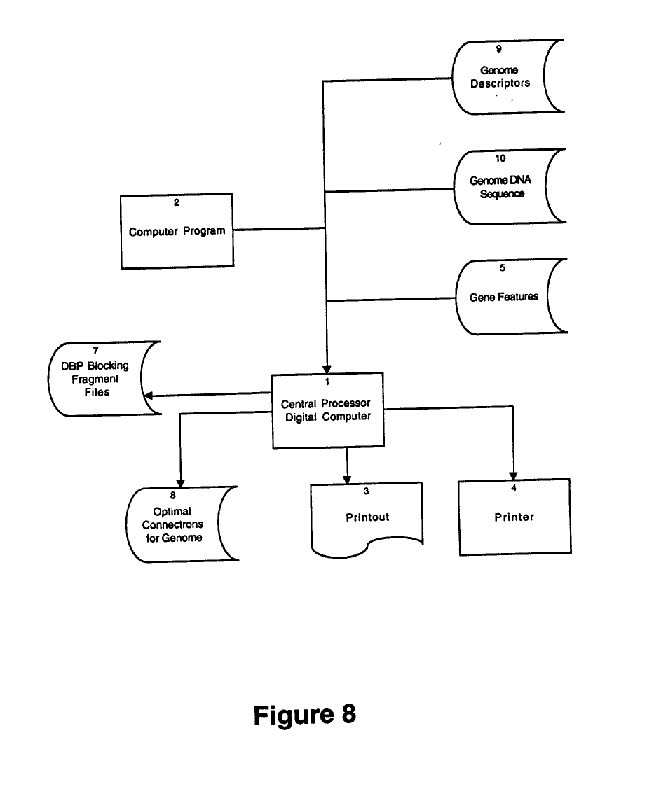

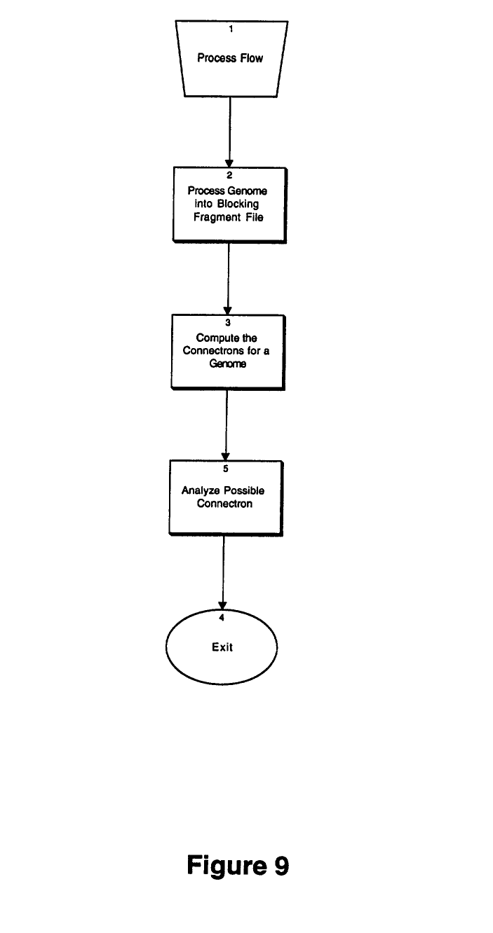

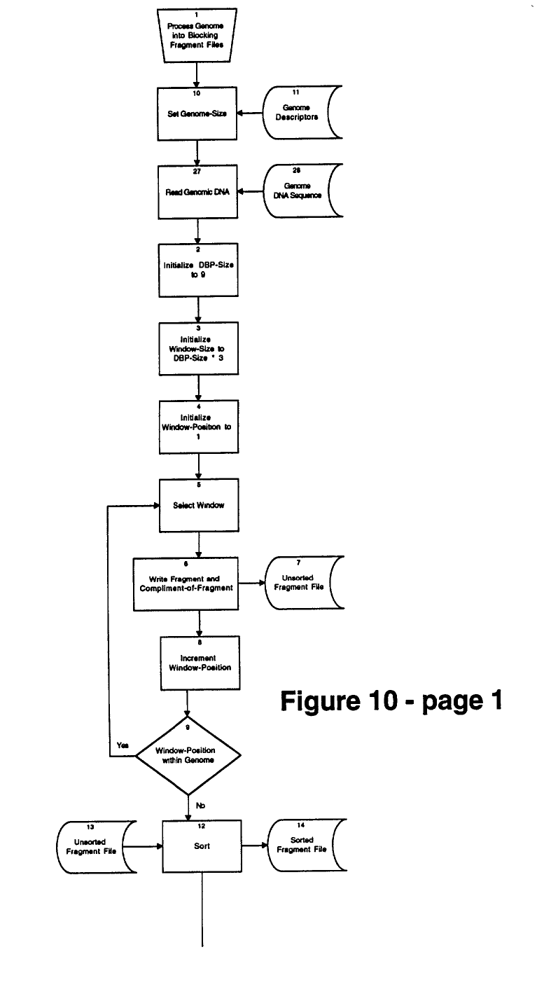

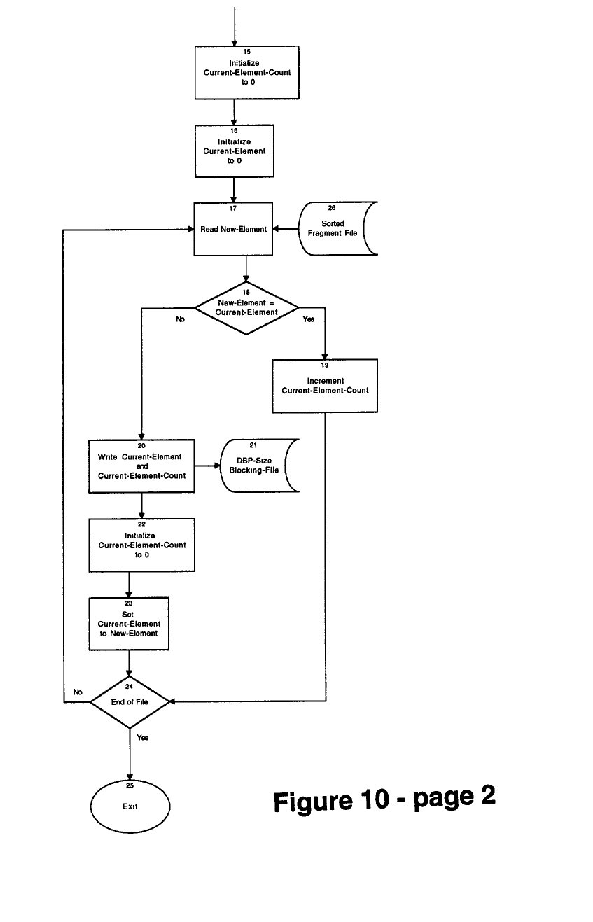

The algorithm for the determination of connectrons in any genome or any genome fragment is represented in the following flow diagrams. The Level 0 diagram in FIG. 8 shows the general relationships in a digital computer. The central processor of the digital computer uses the computer program to take genome descriptors, the genomic DNA sequences and the tables of gene features to produce a file of blocking fragments and a file of the optimal connectrons for the genome. The printer serves to make hard copies of the files and this patent application. The level 1 diagram in FIG. 9 shows the three essential steps in the determination of connectrons. The genome is first processed into a blocking fragment file. Then the blocking fragments are used to compute the connectrons for the genome. Finally the potential connectrons are analyzed to determine if the C1/C2 sequences are in the 3′UTR of a gene. The level 2a diagram in FIG. 10 shows the steps required for the processing of the genome into a file of blocking fragments. The genomic DNA sequence is decomposed into 27-base frames for both the positive and negative strands. These fragments are written to the unsorted fragment file. The fragment file is then sorted is then read and formed into groups of equivalent sequences. The (.blk) file contains the sequence and a pointer to the (.gptr) file which contains the pointers to the position of the fragments in the genomes. The position in the genome includes the chromosome number, the position in the chromosome and the strand (i.e. positive and negative). A sample of these files follows

Sample of the (.blk) file for S. cervesiae

Sample of the (.blk) file for S. cervesiae

| | Number | Pointer |
|---|---|---|
| 27-base fragment | of instances | to (.gptr) file |
| 111111111111111111111111111 | 0 | 1 |
| 111111123244233313332443414 | 1 | 2 |
| 111111141113443133314333341 | 2 | 4 |
| 111111232442333133324434141 | 1 | 5 |
| 111111323311133323144423444 | 2 | 7 |
| 111111332213331341414443413 | 2 | 9 |
| 111111333444112343412323243 | 1 | 10 |
| 111111333444113343412323243 | 9 | 19 |
| 111111411134431333143333414 | 2 | 21 |
| 111111443223134142124434124 | 2 | 23 |
| 111112223234344444443144442 | 2 | 25 |
| 111112244123441122214421213 | 8 | 33 |
| 111112311241114344334134431 | 2 | 35 |
| 111112324423331333244341414 | 1 | 36 |
| 111112344232231344242234342 | 1 | 37 |
| 111112433444244421144134211 | 1 | 38 |
| 111112444311313442332142224 | 1 | 39 |
| 111113131241131114424413231 | 1 | 40 |
| 111113143332344311113133411 | 1 | 41 |
| 111113233111333231444234441 | 2 | 43 |

In fragments above 1 = G, 2 = C, 3 = A, 4 = T

In fragments above 1=G, 2=C, 3=A, 4=T

Sample of the (.gptr) file for S. cervesiae

There are 16 chromosomes in S. cervesiae

Sample of the (.gptr) file for S. cervesiae
There are 16 chromosomes in S. cervesiae

| | | Position | |
|---|---|---|---|
| Item | Chromosome | in Chromosome | Direction |
| 1 | 0 | 0 | 0 |
| 2 | 4 | 11137 | 1 |
| 3 | 12 | 467619 | 1 |
| 4 | 12 | 458482 | 1 |
| 5 | 4 | 11138 | 1 |
| 6 | 12 | 465759 | 2 |
| 7 | 12 | 456622 | 1 |
| 8 | 1 | 219366 | 1 |
| 9 | 8 | 539978 | 1 |
| 10 | 14 | 522451 | 1 |
| 11 | 4 | 1099073 | 1 |
| 12 | 4 | 1210003 | 1 |
| 13 | 7 | 539068 | 1 |
| 14 | 12 | 654136 | 1 |
| 15 | 12 | 596455 | 1 |
| 16 | 15 | 121016 | 1 |
| 17 | 15 | 598127 | 2 |
| 18 | 16 | 847724 | 1 |
| 19 | 16 | 59765 | 1 |
| 20 | 12 | 467620 | 1 |
| 21 | 12 | 458483 | 1 |
| 22 | 12 | 461657 | 1 |
| 23 | 12 | 452520 | 1 |
| 24 | 13 | 838006 | 1 |
| 25 | 15 | 288270 | 1 |
| 26 | 4 | 83593 | 1 |
| 27 | 4 | 992867 | 1 |
| 28 | 6 | 162265 | 1 |
| 29 | 7 | 845687 | 1 |
| 30 | 10 | 531560 | 2 |
| 31 | 15 | 282208 | 1 |
| 32 | 16 | 860418 | 1 |
| 33 | 16 | 572308 | 1 |
| 34 | 12 | 465992 | 1 |
| 35 | 12 | 456855 | 1 |
| 36 | 4 | 11139 | 1 |
| 37 | 8 | 89343 | 1 |
| 38 | 4 | 10302 | 1 |
| 39 | 1 | 19894 | 2 |
| 40 | 16 | 9311 | 1 |
| 41 | 10 | 735203 | 1 |
| 42 | 12 | 465760 | 1 |
| 43 | 12 | 456623 | 1 |

In direction collumn above 1 = positive strand, 2 = negative strand

In direction column above 1=positive strand, 2=negative strand

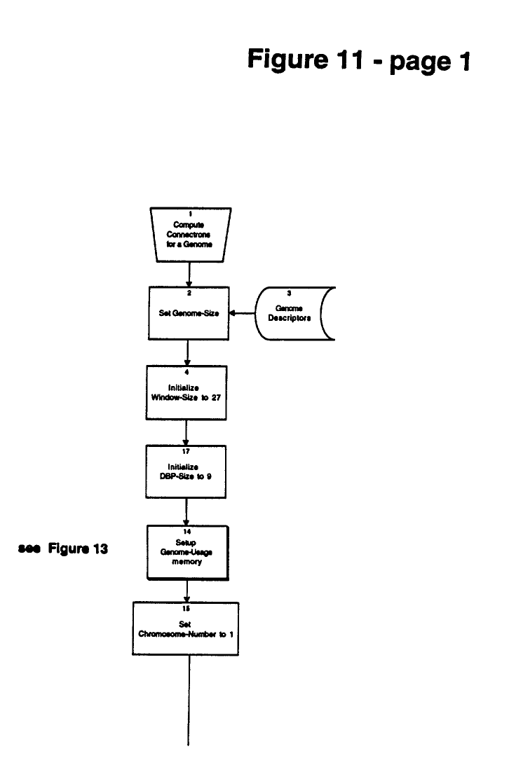

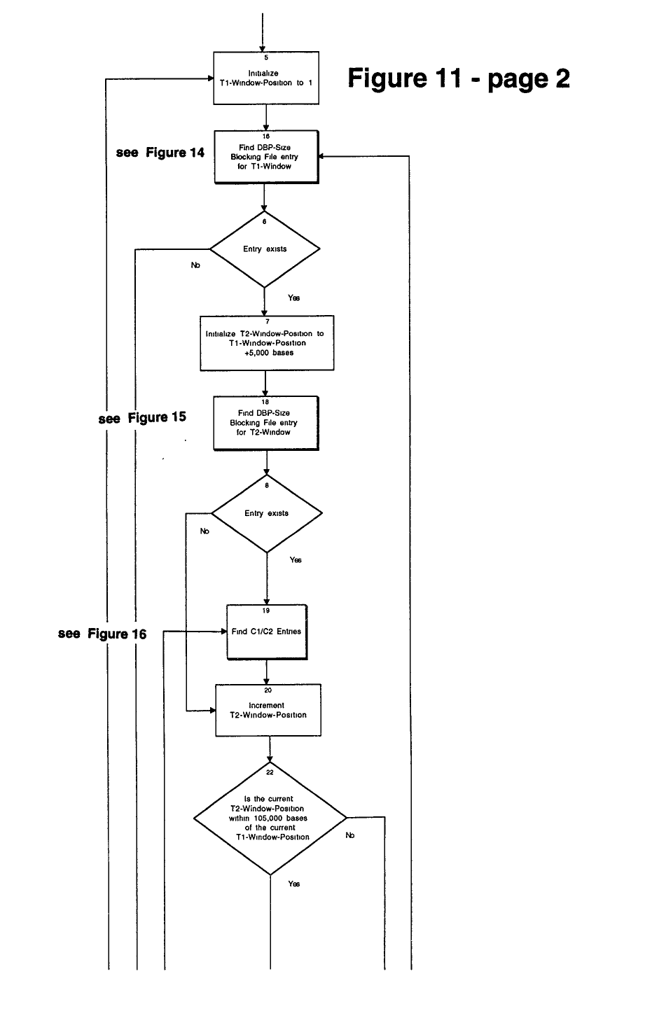

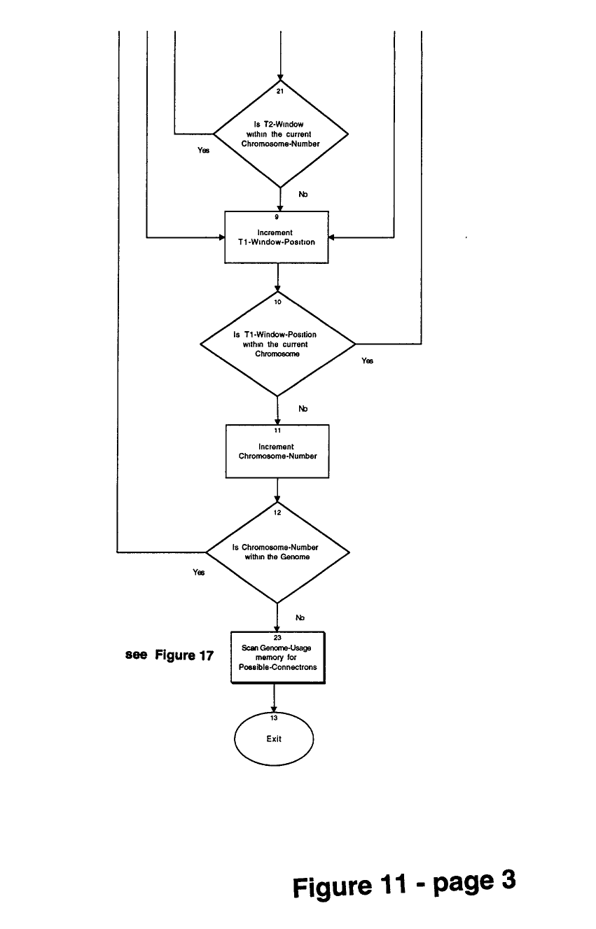

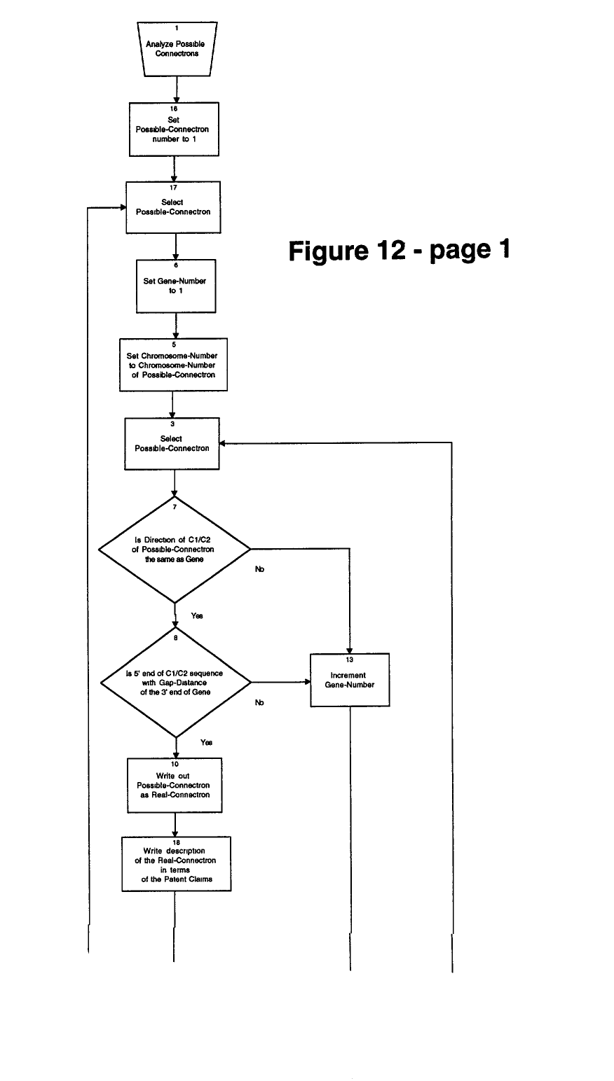

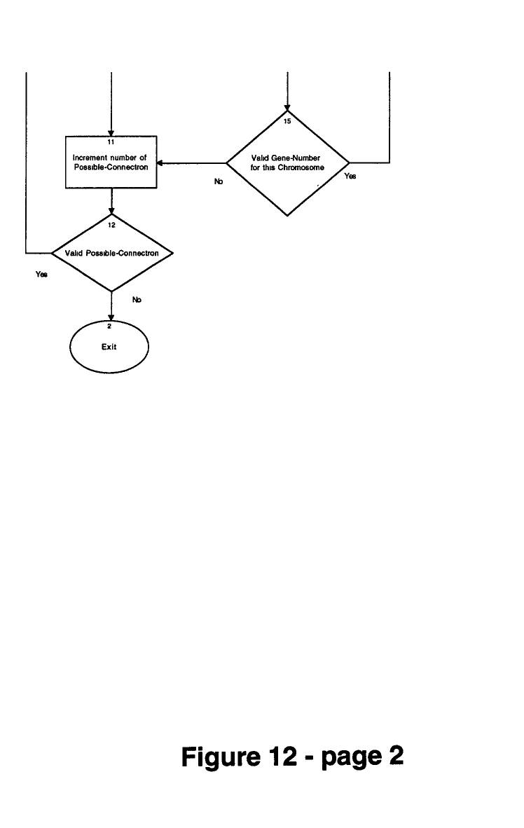

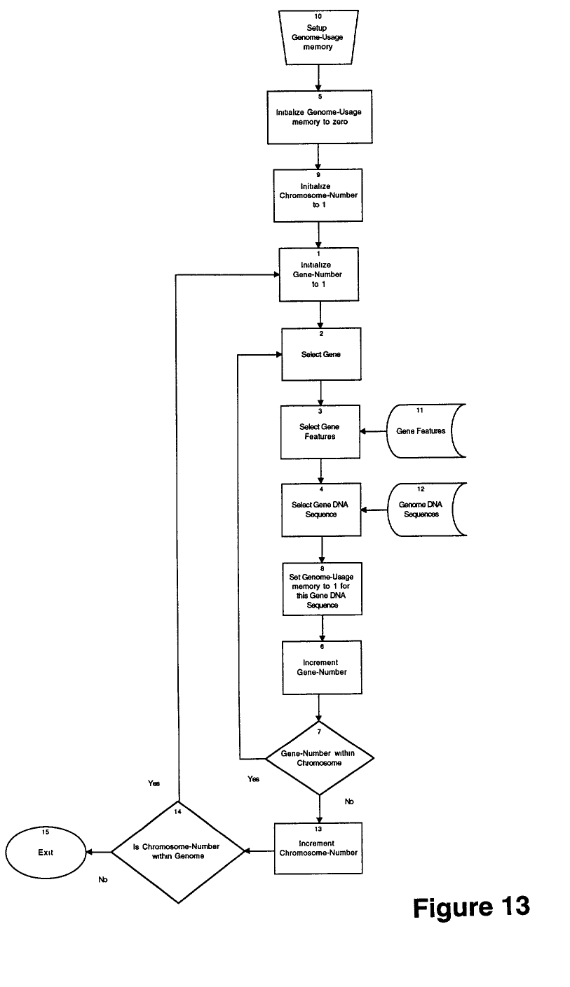

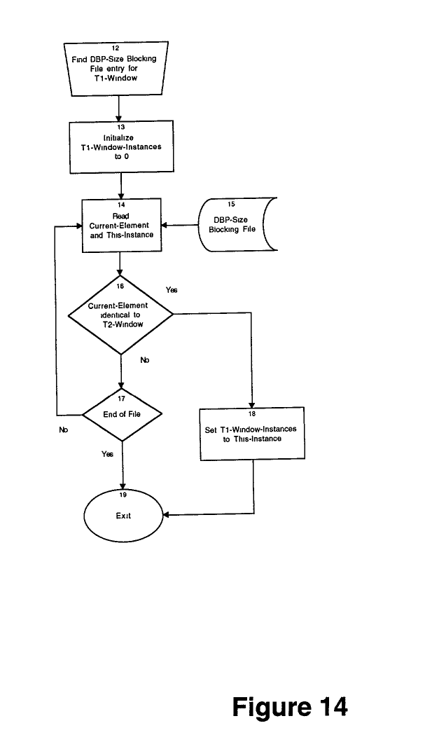

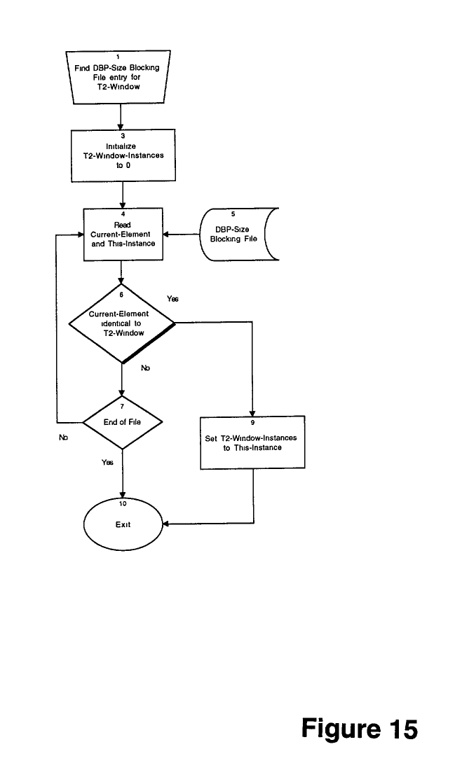

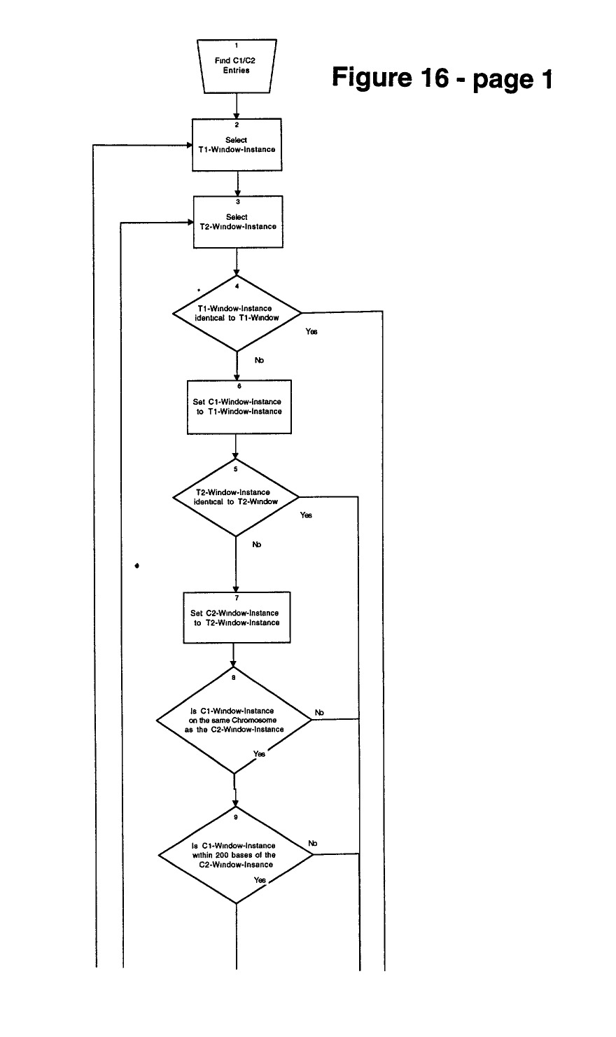

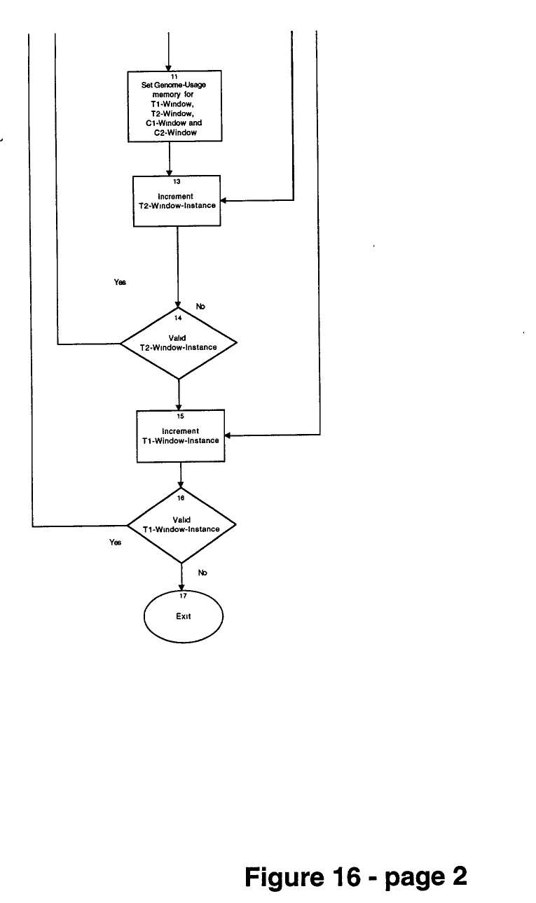

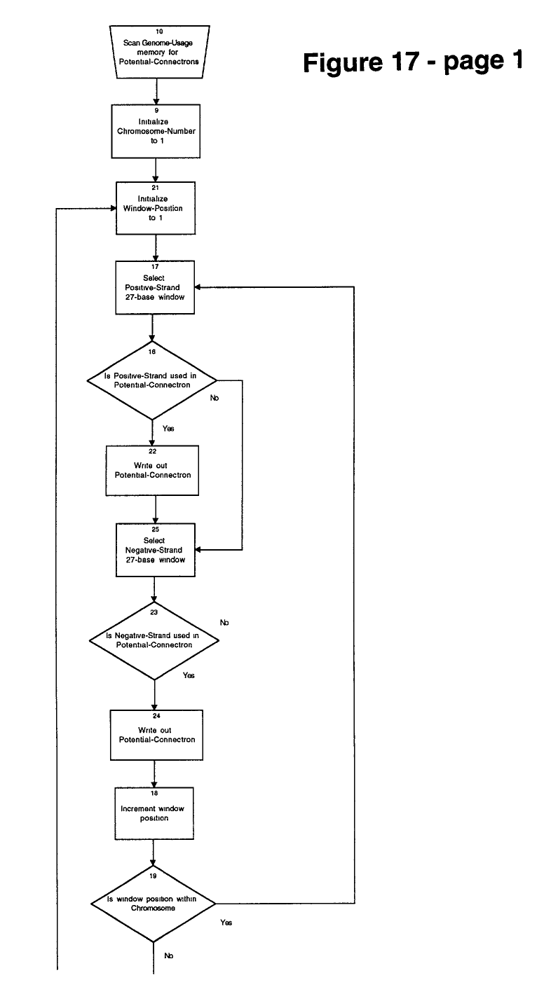

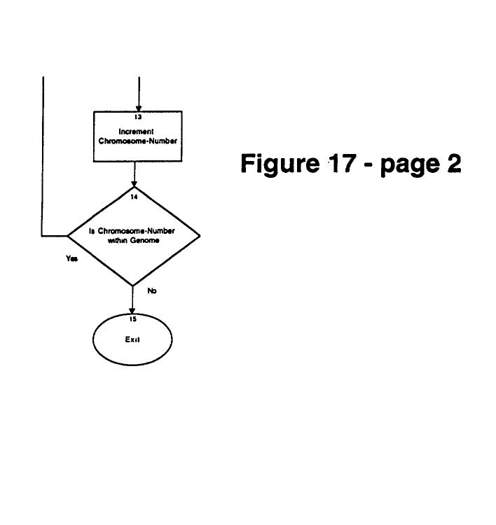

The level 2b diagram in FIG. 11 shows the computation of the connectrons. The genome descriptors consist of the number and length of the chromosomes. The algorithm uses an array that represents several facts about each base position in the genome. The level 3a diagram in FIG. 13 shows the setup of the Genome-Usage memory. The gene features are used to prevent the region of the genome that codes for proteins from being used for the connectron sequences (i.e. the T1s, the T2s, the C1s and the C2s). In the level 2a diagram of FIG. 10, the algorithm steps through each chromosome and within each chromosome through each base position looking for acceptable T1-windows of 27 bases. A T1-window can be used to form a connectron relationship if there are two or more instances of this fragment in the blocking fragment file. The computation in the level 3b diagram of FIG. 14 determines if the T1-window is acceptable of not. Once an acceptable T1-window is found, the algorithm (in the level 2a diagram of FIG. 10) looks for acceptable T2-window positions that lie between 5,000 and 105,000 bases from the T1-window. The computation for determining acceptable T2-window positions is done in the level 3c diagram of FIG. 15. Once a pair of T1 and T2 window positions are found, the algorithm looks among the instances of these T1 and T2 sequences for a pair of sequences C1 and C2 that lie within 200 bases of each other on the same chromosome. The computation for determining acceptable C1/C2 windows is shown in the level 3d diagram in FIG. 16. In the level 3e diagram of FIG. 17 the Genome-Usage memory is scanned for the Possible-Connectrons. In the level 2c diagram of FIG. 12 the Possible-Connectrons are scanned to determine if the C1/C2 sequences are within the Gap-Distance of a gene on either the positive or the negative strands. The Real-Connectrons are then written out in several different files including the descriptions in the claims section.

### EXAMPLES

The algorithm for the determination of optimal connectrons has been applied to a number of different publicly available genomes. The connectron is a tetradic relationship between four sequence elements—T1, T2, C1 and C2. The claims presented in this section are written by the program NearGene that implements the flow diagram Level 2c of FIG. 12. The examples are written a uniform type of English. Each example contains some or all of the following elements

Name of genome

Description of T1

Length of T1 -T2 loop

The chromosome on which the T1-T2 loop exists

The identifier number within the genome of the T1 sequence

The T1 sequence

Description of T2

The identifier number within the genome of the T2 sequence

The T2 sequence

A list of genes whose expression is controlled by the T1-T2 loop

The common names of the genes as obtained from the NCBI gene feature file (.ptt)

A list of C1/C2 short loops whose expression if controlled by the T1-T2 loop

The chromosome on which the C1/C2 short loop exists

The common name of the gene which expresses the C1/C2 short loop as an RNA

The sequence of the C1/C2 short loop

A list of C1/C2 short loops that control the formation of the T1-T2 loop

The chromosome on which the C1/C2 short loop exists

The common name of the gene which expresses the C1/C2 short loop as an RNA

The sequence of the C1/C2 short loop

The match between the C1/C2 sequence and the T1 sequence

The match between the C1/C2 sequence and the T2 sequence

The uniform descriptions make it possible to rapidly comprehend the specifics in each example.

When a sequence element is very long a series of four dots has been inserted between the beginning and ending sequence groups. A variable number of bases have been deleted.

Index of Pages for Connectron Samples

Page 39

1. Connectrons occur in prokaryotes, archea, single-celled eukaryotes and multi-celled eukaryotes.

Page 57

2. Many Connectrons control the expression of one set of genes in prokaryotes, archea, single-celled eukaryotes and multi-celled eukaryotes.

Page 83

3. One connectron controls the expression of many sets of genes in prokaryotes, archea, single-celled eukaryotes and multi-celled eukaryotes.

Page 107

4. Connectrons occur between prokaryotes and their plasmids.

Page 117

5. Connectrons occur in plants and higher animals

Page 126

6. Permanent connectrons exist in prokaryotes, archea, single-celled eukaryotes and multi-celled eukaryotes.

Page 135

7. Transient connectrons exist in prokaryotes, archea, single-celled eukaryotes and multi-celled eukaryotes.

Page 152

8. Self-limiting connectrons occur in prokaryotes, archea, single-celled eukaryotes and multi-celled eukaryotes

Page 164

9. Geneless connectrons exist in single-celled and multi-celled eukaryotes

Page 174

10. One connectron controls many geneless connectrons in single-celled and multi-celled eukaryotes

1. Connectrons Occur in Prokaryotes, Archea, Single-celled Eukaryotes and Multi-celled Eukaryotes.

Connectrons exist as tetradic relationships where the sequence T1 is equivalent to the sequence C1 (written T1=C1) and where the sequence T2 equals the sequence C2 (written T2=C2) where T1 and T2 are DNA sequences 20 or more bases in length, where the C1 sequence is adjacent to the C2 sequence, where the T1 and T2 sequences are on the same chromosome, and where the C1/C2 sequences are on the same chromosome as T1 and T2 or where the C1/C2 sequences are on a chromosome different from T1 and T2. The connectron relationship has been found to exist in prokaryotes, archea, single-celled eukaryotes and multi-celled eukaryotes.

Example of a Prokaryote Connectron—E. coli

In this example the existence of the T1-T2 (3197-3308) long loop is controlled by three C1/C2 short loops (3307, 3432 and 2218). The T1-T2 long loop controls the expression of 64 genes on chromosome 1 in addition to six C1/C2 (3204, 3206, 3223, 3228, 3301 and 3327) short loops. The C1/C2 short loop 3327 lies outside the range of the T1-T2 long loop (3197-3308) but this C1/C2 is expressed as a 3′UTR to the gene hemG that is within the range of the T1-T2 long loop.

```
                                3307 Chromosome 1
                                3432 Chromosome 1
                                2218 Chromosome 1
                                |
*------------------*------------------*
| Chromosome 1 |
3197 3308
| 3204 3206 |
| 3224 3228 |
| 3301 3327 |
--------------------------------------------------------
```

Connectron Control Elements for Chromosome 1 of the E. coli Genome

A double stranded DNA loop of length 93.542 kilo-bases on chromosome 1 is bounded on the left by a T1 sequence whose identifier is 3197. This T1 control element has the DNA sequence

AAAAAATGCGCGGTCAGAAAATTATTTTAAATTTCCTCTTGTCAGGCCGG
AATAACTCCCTATAATGCGCCACCACTGACACGGAACAACGGCAAACACG
CCGCCGGGTCAGCGGGGTTCTCCTGAGAACTCCGGCAGAGAAAGCAAAA
ATAAATGCTTGACTCTGTAGCGGGAA

This double stranded DNA loop is bounded on the right by a T2 control element whose identifier is 3308. This T2 control element has the DNA sequence

TAAATTTCCTCTTGTCAGGCCGGAATAACTCCCTATAATGCGCCACCACTG
ACACGGAACAACGGCAAACACGCCGCCGGGTCAGCGGGGTTCTCCTGAG
AACTCCGGCAGAGAAAGCAAAAATAAATGCTTGACTCTGTAGCGGGAAG
GCGTATTATGCACACCCCGCGCCGCT

This long T1/T2 double stranded DNA loop modulates the expression of the following genes

| | | | | | | | | |
|---|---|---|---|---|---|---|---|---|
| rrsC | gltU | rrlC | rrfC | aspT | trpT | yifA | yifE | yifB |
| ilvL | ilvG_1 | ilvM | ilvE | ilvD | ilvA | ilvY | ilvC | ppiC |
| b3776 | rep | gppA | rhlB | trxA | rhoL | rho | rfe | wzzE |
| wecB | rffH | weeD | wecE | wzxE | yifM_2 | wecG | yifK | |
| argX | hisR | leuT | proM | aslB | aslA | hemY | hemX | |
| hemD | cyaA | cyaY | b3808 | dapF | uvrD | b38l4 | corA | |
| yigF | yigG | rarD | yigI | pldA | recQ | yigJ | yigK | pldB |
| yigL | yigM | metR | metE | ysgA | udp | yigN | ubiE | yigP |
| b3836 | yigU | yigW_1 | rfaH | yigC | ubiB | fadA | fadB | |
| pepQ | trkH | hemG | | | | | | |

This long T1/T2 double stranded DNA loop modulates the expression of the following C1/C2 short loops

A C1/C2 short loop on chromosome 1 whose identifier is 3204 controls the expression of the genes of one or more other T1/T2 long loops. This C1/C2 short loop is expressed as a RNA single strand that is 3′UTR to the gene rrsC and has the DNA sequence

GATGTGCCCAGATGGGATTAGCTAGTAGGTGGGGTAACGGCTCACCTAGG
CGACGATCCCTAGCTGGTCTGAGAGGATGACCAGCCACACTGGAACTGAG
ACACGGTCCAGACTCCTACGGGAGGCAGCAGTGGGGAATATTGCACAATG
GGCGCAAGCCTGATGCAGCCATGCCGCGTGTATGAA

A C1/C2 short loop on chromosome 1 whose identifier is 3206 controls the expression of the genes of one or more other T1/T2 long loops. This C1/C2 short loop is expressed as a RNA single strand that is 3′UTR to the gene rrsC and has the DNA sequence

GTCCCCTTCGTCTAGAGGCCCAGGACACCGCCCTTTCACGGCGGTAACAG
GGGTTCGAATCCCCTAGGGGACGCCACTTGCTGGTTTGTGAGTGAAAGTC
ACCTGCCTTAATATCTCAAAACTCATCTTCGGGTGATGTTTGAGATATTTG
CTCTTTAAAAATCTGGATCAAGCTGAAAATTGAAA

A C1/C2 short loop on chromosome 1 whose identifier is 3223 controls the expression of the genes of one or more other T1/T2 long loops. This C1/C2 short loop is expressed as a RNA single strand that is 3′UTR to the gene rrlC and has the DNA sequence

GCTGAAGTAGGTCCCAAGGGTATGGCTGTTCGCCATTTAAAGTGGTACGC
GAGCTGGGTTTAGAACGTCGTGAGACAGTTCGGTCCCTATCTGCCGTGGG
CGCTGGAGAACTGAGGGGGGCTGCTCCTAGTACGAGAGGACCGGAGTGG
ACGCATCACTGGTGTTCGGGTTGTCATGCCAATGGCA

A C1/C2 short loop on chromosome 1 whose identifier is 3225 controls the expression of the genes of one or more other T1/T2 long loops. This C1/C2 short loop is expressed as a RNA single strand that is 3′UTR to the gene rrlC and has the DNA sequence

AAACAGAATTTGCCTGGCGGCCGTAGCGCGGTGGTCCCACCTGACCCCAT
GCCGAACTCAGAAGTGAAACGCCGTAGCGCCGATGGTAGTGTGGGGTCTC
CCCATGCGAGAGTAGGGAACTGCCAGGCATCAAATTAAGCAGTA

A C1/C2 short loop on chromosome 1 whose identifier is 3228 controls the expression of the genes of one or more other T1/T2 long loops. This C1/C2 short loop is expressed as a RNA single strand that is 3′UTR to the gene rrfC and has the DNA sequence

GGTCATAAAACCGGTGGTTGTAAAAGAATTCGGTGGAGCGGTAGTTCAGT
CGGTTAGAATACCTGCCTGTCACGCAGGGGGTCGCGGGTTCGAGTCCCGT
CCGTTCCGCCAC

A C1/C2 short loop on chromosome 1 whose identifier is 3301 controls the expression of the genes of one or more other T1/T2 long loops. This C1/C2 short loop is expressed as a RNA single strand that is 3′UTR to the gene ubiB and has the DNA sequence

TTATCGTGCCTACAAATAGTCCGAACCGTAGGCCGGATAAGGCGTTTACG
CCGCATC

A C1/C2 short loop on chromosome 1 whose identifier is 3307 controls the expression of the genes of one or more other T1/T2 long loops. This C1/C2 short loop is expressed as a RNA single strand that is 3′UTR to the gene fadA and has the DNA sequence

TGCCGGATGCGGCGTAAACGCCTTATCCGGCCTACGGTTCGGACTATTTGT
AGGCA

A C1/C2 short loop on chromosome 1 whose identifier is 3327 controls the expression of the genes of one or more other T1/T2 long loops. This C1/C2 short loop is expressed as a RNA single strand that is 3′UTR to the gene hemG and has the DNA sequence

AAAAAATGCGCGGTCAGAAAATTATTTTAAATTTCCTCTTGTCAGGCCGG
AATAACTCCCTATAATGCGCCACCACTGACACGGAACAACGGCAAACACG
CCGCCGGGTCAGCGGGGTTCTCCTGAGAACTCCGGCAGAGAAAGCAAAA
ATAAATGCTTGACTCTGTAGCGGGAAGGCGTATTATG...CCCGTCACACCA
TGGGAGTGGGTTGCAAAAGAAGTAGGTAGCTTAACCTTCGGGAGGGCGCT
TACCACTTTGTGATTCATGACTGGGGTGAAGTCGTAACAAGGTAACCGTA
GGGGAACCTGCGGTTGGATCACCTCCTTACCTTAAAGAAGCGTTCTTTG

The expression of genes in this T1/T2 long loop is controlled by the following C1/C2 short loops.

A C1/C2 short loop on chromosome 1 whose identifier is 3307 controls the expression of the genes in this T1/T2 long loop. This C1/C2 short loop is expressed as a RNA single strand that is 3′UTR to the gene hemG and has the DNA sequence

AAAAAATGCGCGGTCAGAAAATTATTTTAAATTTCCTCTTGTCAGGCCGG
AATAACTCCCTATAATGCGCCACCACTGACACGGAACAACGGCAAACACG
CCGCCGGGTCAGCGGGGTTCTCCTGAGAACTCCGGCAGAGAAAGCAAAA
ATAAATGCTTGACTCTGTAGCGGGAAGGCGTATTATG...CCCGTCACACCA
TGGGAGTGGGTTGCAAAAGAAGTAGGTAGCTTAACCTTCGGGAGGGCGCT
TACCACTTTGTGATTCATGACTGGGGTGAAGTCGTAACAAGGTAACCGTA
GGGGAACCTGCGGTTGGATCACCTCCTTACCTTAAAGAAGCGTTCTTTG

The match between the T1 sequence and the C1/C2 sequence is

AAAAAATGCGCGGTCAGAAAATTATTTTAAATTTCCTCTTGTCAGGCCGG
AATAACTCCCTATAATGCGCCACCACTGACACGGAACAACGGCAAACACG
CCGCCGGGTCAGCGGGGTTCTCCTGAGAACTCCGGCAGAGAAAGCAAAA
ATAAATGCTTGACTCTGTAGCGGGAA

The match between the T2 sequence and the C1/C2 sequence is

TAAATTTCCTCTTGTCAGGCCGGAATAACTCCCTATAATGCGCCACCACTG
ACACGGAACAACGGCAAACACGCCGCCGGGTCAGCGGGGTTCTCCTGAG
AACTCCGGCAGAGAAAGCAAAAATAAATGCTTGACTCTGTAGCGGGAAG
GCGTATTATGCACACCCCGCGCCGCT

A C1/C2 short loop on chromosome 1 whose identifier is 3432 controls the expression of the genes in this T1/T2 long loop. This C1/C2 short loop is expressed as a RNA single strand that is 3′UTR to the gene btuB and has the DNA sequence

TGCGCGGTCAGAAAATTATTTTAAATTTCCTCTTGTCAGGCCGGAATAACT
CCCTATAATGCGCCACCACTGACACGGAACAACGGCAAACACGCCGCCGG
GTCAGCGGGGTTCTCCTGAGAACTCCGGCAGAGAAAGCAAAAATAAATG
CTTGACTCTGTAGCGGGAAGGCGTATTATGCACACC...ACACCATGGGAGT
GGGTTGCAAAAGAAGTAGGTAGCTTAACCTTCGGGAGGGCGCTTACCACT
TTGTGATTCATGACTGGGGTGAAGTCGTAACAAGGTAACCGTAGGGGAAC
CTGCGGTTGGATCACCTCCTTACCTTAAAGAAGCGT

The match between the T1 sequence and the C1/C2 sequence is

TGCGCGGTCAGAAAATTATTTTAAATTTCCTCTTGTCAGGCCGGAATAACT
CCCTATAATGCGCCACCACTGACACGGAACAACGGCAAACACGCCGCCGG
GTCAGCGGGGTTCTCCTGAGAACTCCGGCAGAGAAAGCAAAAATAAATG
CTTGACTCTGTAGCGGGAA

The match between the T2 sequence and the C1/C2 sequence is

TAAATTTCCTCTTGTCAGGCCGGAATAACTCCCTATAATGCGCCACCACTG
ACACGGAACAACGGCAAACACGCCGCCGGGTCAGCGGGGTTCTCCTGAG
AACTCCGGCAGAGAAAGCAAAAATAAATGCTTGACTCTGTAGCGGGAAG
GCGTATTATGCACACCCCGCGCCGCT

A C1/C2 short loop on chromosome 1 whose identifier is 2218 controls the expression of the genes in this T1/T2 long loop. This C1/C2 short loop is expressed as a RNA single strand that is 3′UTR to the gene clpB and has the DNA sequence

CTTGTCAGGCCGGAATAACTCCCTATAATGCGCCACCACTGACACGGAAC
AACGGCAAACACGCCGCCGGGC

The match between the T1 sequence and the C1/C2 sequence is

CTTGTCAGGCCGGAATAACTCCCTATAATGCGCCACCACTGACACGGAAC
AACGGCAAACACGCCGCCGGGC

The match between the T2 sequence and the C1/C2 sequence is

CTTGTCAGGCCGGAATAACTCCCTATAATGCGCCACCACTGACACGGAAC
AACGGCAAACACGCCGCCGGGTC

Example of an Archea Connectron—H. pylori

In this example the existence of the T1-T2 (812-882) long loop is controlled by three C1/C2 short loops (881, 813 and 1214). The T1-T2 long loop controls the expression of 54 genes on chromosome 1 in addition to one C1/C2 (843) short loop.

```
                                881 Chromosome 1
                                813 Chromosome 1
                                1241 Chromosome 1
                                |
*------------------*------------------*
| Chromosome 1 |
812 882
| 842 |
--------------------------------------------------------
```

Connectron control elements for chromosome 1 of H. pylori genome

A double stranded DNA loop of length 96.385 kilo-bases on chromosome 1 is bounded on the left by a T1 sequence whose identifier is 812. This T1 control element has the DNA sequence

TTTTACTCATAGGGTTTTTATAGTTCCTAGCGGAACTAAAGCA

This double stranded DNA loop is bounded on the right by a T2 control element whose identifier is 882. This T2 control element has the DNA sequence

TAGCGGAACTAAAGCATTCATCCCAAACACTAAAGATATTTGG

This long T1/T2 double stranded DNA loop modulates the expression of the following genes

| | | | | | | |
|---|---|---|---|---|---|---|
| HP0999 | HP1000 | HP1001 | HP1002 | HP1003 | HP1005 | HP1006 |
| HP1008 | HP1009 | HPtRNA-Pro | HP1010 | HP1011 | HP1013 | HP1015 |
| HP1017 | HP1018 | HP1020 | HP1021 | HP1022 | HP1023 | HP1024 |
| HP1025 | HP1027 | HP1028 | HP1030 | HP1031 | HP1033 | HP1034 |
| HP1038 | HP1039 | HP1040 | HP1041 | HP1042 | HP1043 | HP1044 |
| HP1045 | HP1046 | HP1051 | HP1052 | HP1055 | HP1056 | HP1058 |
| HP1060 | HP1065 | HPtRNA-Ser | HP1066 | HP1067 | HP1069 | HP1070 |
| HP1074 | HP1075 | HP1076 | HP1077 | HP1078 | HP1079 | HP1080 |
| HP1081 | HP1083 | HP1084 | HP1085 | HP1088 | HP1091 | HP1092 |
| HP1093 | HP1094 | HP1095 | HP1096 | | | |

This long T1/T2 double stranded DNA loop modulates the expression of the following C1/C2 short loops

A C1/C2 short loop on chromosome 1 whose identifier is 813 controls the expression of the genes of one or more other T1/T2 long loops. This C1/C2 short loop is expressed as a RNA single strand that is 3′UTR to the gene HP0998 and has the DNA sequence

TTTTACTCATAGGGTTTTTATAGTTCCTAGCGGAACTAAAGCATTCATCCC
AAACACTAAAGATATTTGG

The expression of genes in this T1/T2 long loop is controlled by the following C1/C2 short loops.

A C1/C2 short loop on chromosome 1 whose identifier is 881 controls the expression of the genes of one or more other T1/T2 long loops. This C1/C2 short loop is expressed as a RNA single strand that is 3′UTR to the gene HP1096 and has the DNA sequence

TTTTACTCATAGGGTTTTTATAGTTCCTAGCGGAACTAAAGCATTCATCCC
AAACACTAAAGATATTTGG

The match between the T1 sequence and the C1/C2 sequence is

TTTTACTCATAGGGTTTTTATAGTTCCTAGCGGAACTAAAGCA

The match between the T2 sequence and the C1/C2 sequence is

TAGCGGAACTAAAGCATTCATCCCAAACACTAAAGATATTTGG

The expression of genes in this T1/T2 long loop is controlled by the following C1/C2 short loops.

A C1/C2 short loop on chromosome 1 whose identifier is 813 controls the expression of the genes in this T1/T2 long loop. This C1/C2 short loop is expressed as a RNA single strand that is 3′UTR to the gene HP0998 and has the DNA sequence

TTTTACTCATAGGGTTTTTATAGTTCCTAGCGGAACTAAAGCATTCATCCC
AAACACTAAAGATATTTGG

A C1/C2 short loop on chromosome 1 whose identifier is 881 controls the expression of the genes in this T1/T2 long loop. This C1/C2 short loop is expressed as a RNA single strand that is 3′UTR to the gene HP1096 and has the DNA sequence

TTTTACTCATAGGGTTTTTATAGTTCCTAGCGGAACTAAAGCATTCATCCC
AAACACTAAAGATATTTGG

The match between the T1 sequence and the C1/C2 sequence is

TTTTACTCATAGGGTTTTTATAGTTCCTAGCGGAACTAAAGCA

The match between the T2 sequence and the C1/C2 sequence is

TAGCGGAACTAAAGCATTCATCCCAAACACTAAAGATATTTGG

A C1/C2 short loop on chromosome 1 whose identifier is 1241 controls the expression of the genes in this T1/T2 long loop. This C1/C2 short loop is expressed as a RNA single strand that is 3′UTR to the gene HP1535 and has the DNA sequence

TTTTACTCATAGGGTTTTTATAGTTCCTAGCGGAACTAAAGCATTCATCCC
AAACA

The match between the T1 sequence and the C1/C2 sequence is

TTTTACTCATAGGGTTTTTATAGTTCCTAGCGGAACTAAAGCA

The match between the T2 sequence and the C1/C2 sequence is

TAGCGGAACTAAAGCATTCATCCCAAACA

Example of Single-celled Connectron—S. cervesiae

In this example the existence of the T1-T2 (1352-1416) long loop on chromosome 4 is controlled by one C1/C2 short loop (4213) on chromosome 10. The T1-T2 long loop controls the expression of 34 genes on chromosome 4 in addition to one C1/C2 (1356) short loop.

```
                                  4213 Chromosome 10
                                  |
*------------------*------------------*
| Chromosome 4 |
1352 1416
| 1356 |
--------------------------------------------------------
```

Connectron Control Elements for Chromosome 1 of S. cervesiae Genome

A double stranded DNA loop of length 68.908 kilo-bases on chromosome 4 is bounded on the left by a T1 sequence whose identifier is 1352. This T1 control element has the DNA sequence

TTATGAGAAGCTGTCATCGAAGTTAGAGGAAGCTGAA

This double stranded DNA loop is bounded on the right by a T2 control element whose identifier is 1416. This T2 control element has the DNA sequence

ATTAGATCTATTACATTATGGGTGGTATGTTGGAATAAAAATCAACTATCA
TCTACTAACTAGTATTTACGTTACTAGTATATTATCATATACGGTGTTAGA
AGATGACGCAAATGATGAGAAATAGTCATCTAAATTAGTGGAAGCTGAA
ACGCAAGGATTGATAATGTAATAGGATCAATGAATATTAACATATAAAAC
GATGATAATAATATTTATAGAATTGTGTAGAATTGCAGATTCCCTTTTATG
GATTCCTAAATCCTTGAGGAGAACTTCTAGTATATCTACATACCTAATATT
ATAGCCTTAATCACAATGGAATCCCAACAATTACATCAAAATCCACATTC
TCTACAGTA

This long T1/T2 double stranded DNA loop modulates the expression of the following genes

YDR170W-A YDR171W YDR172W YDR173C YDR174W YDR175C
YDR176W YDR177W YDR178W YDR179C YDR179W-A YDR180W
YDR181C YDR182W YDR183W YDR184C YDR185C YDR186C
YDR187C YDR188W YDR189W YDR190C YDR191W YDR192C
YDR193W YDR194C YDR195W YDR196C YDR197W YDR198C
YDR199W YDR200C YDR201W YDR202C YDR203W YDR204W
YDR205W YDR206W YDR207C YDR208W YDR209C YDR210W

This long T1/T2 double stranded DNA loop modulates the expression of the following C1/C2 short loops

A C1/C2 short loop on chromosome 4 whose identifier is 1356 controls the expression of the genes of one or more other T1/T2 long loops. This C1/C2 short loop is expressed as a RNA single strand that is 3′UTR to the gene YDR170W-A and has the DNA sequence

AATCACACTAATCATTCTGATGATGAACTCCCTGGACACCTCCTTCTCGAT
TCAGGAGCATCACGAACCCTTATAAGATCTGCTCATCACATACACTCAGC
ATCATCTAATCCTGACATAAACGTAGTTTGATGCTCAAAAAAGAAATATAC
CAATTAACGCTATTGGTGACCTACAATTCACTTCCAGGACAACACCAAA
ACATCAATAAAGGTATTGCACACTCCTAACATAGCCTATGACTTACTCAGT
TTGAATGAATTGGCTGCAGTAGATATCACAGCATGCTTTACCAAAAACGT
CTTAGAACG

The expression of genes in this T1/T2 long loop is controlled by the following C1/C2 short loops.

A C1/C2 short loop on chromosome 10 whose identifier is 4213 controls the expression of the genes in this T1/T2 long loop. This C1/C2 short loop is expressed as a RNA single strand that is 3′UTR to the gene YJR029W and has the DNA sequence

ATCTATTACATTATGGGTGGTATGTTGGAATAAAAATCCACTATCGTCTAT
CAACTAATAGTTATATTATCAATATATTATCATATACGGTGTTAAGATGAT
GACATAAGTTATGAGAAGCTGTCATCGAAGTTAGAGGAAGCTGAAACGC
AAGGATTGATAATGTAATAGGATCAATGAATATAAACATATAAAACGGA
ATGAGGAATAATCGTAATATTAGTATGTAGAAATATAGATTCCATTTTGA
GGATTCCTATATCCTCGAGGAGAACTTCTAGTATATTCTGTATACCTAATA
TTATAGCCTTTATCAACAATGGAATCCCAACAATTATCTCAACAT

The match between the T1 sequence and the C1/C2 sequence is

TTATGAGAAGCTGTCATCGAAGTTAGAGGAAGCTGAA

The match between the T2 sequence and the C1/C2 sequence is

ATCTATTACATTATGGGTGGTATGTTGGAATAAAAATC

Example of a Multi-celled Connectron—C. elegans

In this example the existence of the T1-T2 (9-138) long loop on chromosome 1 is controlled by three C1/C2 short loops on chromosome 5 (21719, 21949 and 21655). The T1-T2 long loop controls the expression of four genes on chromosome 1 in addition to seven C1/C2 (119, 122, 125, 130, 132, 134 and 136) short loops.

```
                                21719 Chromosome 5
                                21949 Chromosome 5
                                21655 Chromosome 5
                                |
*------------------*------------------*
| Chromosome 1 |
95 138
| 119 122 |
| 125 130 |
| 132 134 |
| 136 |
--------------------------------------------------------
```

A double stranded DNA loop of length 41.978 kilo-bases on chromosome 1 is bounded on the left by a T1 sequence whose identifier is 95. This T1 control element has the DNA sequence

CAGCACGTTCTTAACCATGCAAAATCAGTTGAGAACTCTGCGTCTCTTCTC
CCGC

This double stranded DNA loop is bounded on the right by a T2 control element whose identifier is 138. This T2 control element has the DNA sequence

ACTCTGCGTCTCTTCTCCCGCATTTTTTGTAGATCA

This long T1/T2 double stranded DNA loop modulates the expression of the following genes

Y73A3A.1 Y73A3A.1 ZC123.3 ZC123.2

This long T1/T2 double stranded DNA loop modulates the expression of the following C1/C2 short loops

A C1/C2 short loop on chromosome 1 whose identifier is 119 controls the expression of the genes of one or more other T1/T2 long loops. This C1/C2 short loop is expressed as a RNA single strand that is 3′UTR to the gene ZC123.3 and has the DNA sequence

TTGAGAACTCTGCGTCTCAACTCCCGCATTTTTTGTAGATCTACGTAGATC
AAACCGAAATGGGACACT

A C1/C2 short loop on chromosome 1 whose identifier is 122 controls the expression of the genes of one or more other T1/T2 long loops. This C1/C2 short loop is expressed as a RNA single strand that is 3′UTR to the gene ZC123.3 and has the DNA sequence

GCACGGGGTTCTGGCCTTCCTCATTGAATTTTTCGCGCTCCATTGACAATC
GCCTGCCGGACAACGCGTGGGAAAGTCGTGTACTCCAC

A C1/C2 short loop on chromosome 1 whose identifier is 125 controls the expression of the genes of one or more other T1/T2 long loops. This C1/C2 short loop is expressed as a RNA single strand that is 3′UTR to the gene ZC123.3 and has the DNA sequence

ACGCGCCGTAAATCTACCCCAGATATGGCCGAGCCAAAATGGCCTAGTTTC
GGCAAACTCTTTCATTTCAATTTATGAGGGAAGCCAGAA

A C1/C2 short loop on chromosome 1 whose identifier is 130 controls the expression of the genes of one or more other T1/T2 long loops. This C1/C2 short loop is expressed as a RNA single strand that is 3′UTR to the gene ZC123.2 and has the DNA sequence

CTCCCGCATTTTTTGTAGATCTACGTAGATCAAACCGAAATGAGGCACTTT
CTGAATCCACGAGCTAGGCTTAAGCTTAGGCTTAAGCTTAGGCCTTTTCTC
AGGCTTAGGCTTAGGCTTA

A C1/C2 short loop on chromosome 1 whose identifier is 132 controls the expression of the genes of one or more other T1/T2 long loops. This C1/C2 short loop is expressed as a RNA single strand that is 3′UTR to the gene ZC123.2 and has the DNA sequence

GCTTATGCTTGGGCTTAGGCTTAGGCGTAGGCTTAGGCTTAGGCTTAGGCT
TATGCTTAGACTTAGTCTCACTATCAGTCTTAGGCTTAGGCTTAGACTTAG
GCTTAAGCTTAGGCTTAAGCTTAGACTTAGGCTTAGGCTTAGGCTTAGGCT
TAGGCTTAGGTTTGGGCTTAGGCTTAGGCTTAACCTC

A C1/C2 short loop on chromosome 1 whose identifier is 134 controls the expression of the genes of one or more other T1/T2 long loops. This C1/C2 short loop is expressed as a RNA single strand that is 3′UTR to the gene ZC123.2 and has the DNA sequence

TCTGCGTCTTTTCTCCCGCATTTTTTGTAGATCTACGTAGATCAAACCGAA
ATGAGGCACTTTCTGAATCCACGAGCTAGGCTTAAGCTTAGGCTTAAGCTT
AGGCCTTTTCTCAGGCTTAGGCTTAGGCTTA

A C1/C2 short loop on chromosome 1 whose identifier is 136 controls the expression of the genes of one or more other T1/T2 long loops. This C1/C2 short loop is expressed as a RNA single strand that is 3′UTR to the gene ZC 123.2 and has the DNA sequence

GCTTATGCTTGGGCTTAGGCTTAGGCGTAGGCTTAGGCTTAGGCTTAGGCT
TATGCTTAGACTTAGTCTCACTATCAGTCTTAGGCTTAGGCTTAGACTTAG
GCTTAAGCTTAGGCTTAAGCTTAGACTTAGGCTTAGGCTTAGGCTTAGGCT
TAGGCTTAGGTTTGGGCTTAGGCTTAGGCTTAACCTC

The expression of genes in this T1/T2 long loop is controlled by the following C1/C2 short loops.

A C1/C2 short loop on chromosome 5 whose identifier is 21719 controls the expression of the genes in this T1/T2 long loop. This C1/C2 short loop is expressed as a RNA single strand that is 3′UTR to the gene C39F7.5 and has the DNA sequence

ACGTTCTTAACCATGCAAAATCAGTTGAGAACTCTGCGTCTCTTCTCCCGC
ATTTTTTGTAGATC

The match between the T1 sequence and the C1/C2 sequence is

ACGTTCTTAACCATGCAAAATCAGTTGAGAACTCTGCGTCTCTTCTCCCGC

The match between the T2 sequence and the C1/C2 sequence is

ACTCTGCGTCTCTTCTCCCGCATTTTTTGTAGATC

A C1/C2 short loop on chromosome 5 whose identifier is 21949 controls the expression of the genes in this T1/T2 long loop. This C1/C2 short loop is expressed as a RNA single strand that is 3′UTR to the gene F136B4.4 and has the DNA sequence

ACCATGCAAAATCAGTTGAGAACTCTGCGTCTCTTCTCCCGCATTTTTTGT
AGATCTACGTAGATCAAGCCGAAATGAGACACTCTGACACCACG

The match between the T1 sequence and the C1/C2 sequence is

ACCATGCAAAATCAGTTGAGAACTCTGCGTCTCTTCTCCCGC

The match between the T2 sequence and the C1/C2 sequence is

ACTCTGCGTCTCTTCTCCCGCATTTTTTGTAGATC

A C1/C2 short loop on chromosome 5 whose identifier is 21655 controls the expression of the genes in this T1/T2 long loop. This C1/C2 short loop is expressed as a RNA single strand that is 3′UTR to the gene C39F7.3 and has the DNA sequence

AACCATGCAAAATCAGTTGAGAACTCTGCGTCTCTTCTCCCGCATTTTTTG
TAGATCTACG

The match between the T1 sequence and the C1/C2 sequence is

AACCATGCAAAATCAGTTGAGAACTCTGCGTCTCTTCTCCCGC

The match between the T2 sequence and the C1/C2 sequence is

ACTCTGCGTCTCTTCTCCCGCATTTTTTGTAGATC

2. Many Connectrons Control the Expression of one Set of Genes in Prokaryotes, Archea, Single-celled Eukaryotes and Multi-celled Eukaryotes.

Many different C1/C2 short loops can control the existence of one T1-T2 long loop. The C1/C2 short loops can be on the same chromosome or on different chromosomes from the T1-T2 long loop. This relationship is described as “many-to-one”. This relationship exists in prokaryotes, archea, single-celled eukaryotes and multi-celled eukaryotes

Example of a Many-to-one Connectron in Prokaryotes—E. coli

In this example the existence of the T1-T2 (3197-3308) long loop is controlled by three C1/C2 short loops (3307, 3432 and 2218).

```
                                3307 Chromosome 1
                                3432 Chromosome 1
                                2218 Chromosome 1
                                |
*------------------*------------------*
| Chromosome 1 |
3197 3308
--------------------------------------------------------
```

A double stranded DNA loop of length 93.542 kilo-bases on chromosome 1 is bounded on the left by a T1 sequence whose identifier is 3197. This T1 control element has the DNA sequence

AAAAAATGCGCGGTCAGAAAATTATTTTAAATTTCCTCTTGTCAGGCCGG
AATAACTCCCTATAATGCGCCACCACTGACACGGAACAACGGCAAACACG
CCGCCGGGTCAGCGGGGTTCTCCTGAGAACTCCGGCAGAGAAAGCAAAA
ATAAATGCTTGACTCTGTAGCGGGAA

This double stranded DNA loop is bounded on the right by a T2 control element whose identifier is 3308. This T2 control element has the DNA sequence

TAAATTTCCTCTTGTCAGGCCGGAATAACTCCCTATAATGCGCCACCACTG
ACACGGAACAACGGCAAACACGCCGCCGGGTCAGCGGGGTTCTCCTGAG
AACTCCGGCAGAGAAAGCAAAAATAAATGCTTGACTCTGTAGCGGGAAG
GCGTATTATGCACACCCCGCGCCGCT

This long T1/T2 double stranded DNA loop modulates the expression of the following genes

| | | | | | | | | |
|---|---|---|---|---|---|---|---|---|
| rrsC | gltU | rrlC | rrfC | aspT | trpT | yifA | yifE | yifB |
| ilvL | ilvG_1 | ilvM | ilvE | ilvD | ilvA | ilvY | ilvC | ppiC |
| b3776 | rep | gppA | rhlB | trxA | rhoL | rho | rfe | wzzE |

| | | | | | | | |
|---|---|---|---|---|---|---|---|
| wecB | rffH | wecD | wecE | wzxE | yifM_2 | wecG | yifK |
| argX | hisR | leuT | proM | aslB | aslA | hemY | hemX |
| hemD | cyaA | cyaY | b3808 | dapF | uvrD | b3814 | corA |

| | | | | | | | | |
|---|---|---|---|---|---|---|---|---|
| yigF | yigG | rarD | yigI | pldA | recQ | yigJ | yigK | pldB |
| yigL | yigM | metR | metE | ysgA | udp | yigN | ubiE | yigP |

| | | | | | | | |
|---|---|---|---|---|---|---|---|
| b3836 | yigU | yigW_1 | rfaH | yigC | ubiB | fadA | fadB |
| pepQ | trkH | hemG | | | | | |

The expression of genes in this T1/T2 long loop is controlled by the following C1/C2 short loops.

A C1/C2 short loop on chromosome 1 whose identifier is 3307 controls the expression of the genes in this T1/T2 long loop. This C1/C2 short loop is expressed as a RNA single strand that is 3′UTR to the gene hemG and has the DNA sequence

AAAAAATGCGCGGTCAGAAAATTATTTTAAATTTCCTCTTGTCAGGCCGG
AATAACTCCCTATAATGCGCCACCACTGACACGGAACAACGGCAAACACG
CCGCCGGGTCAGCGGGGTTCTCCTGAGAACTCCGGCAGAGAAAGCAAAA
ATAAATGCTTGACTCTGTAGCGGGAAGGCGTATTATG...GGAGTCTGCAAC
TCGACTCCATGAAGTCGGAATCGCTAGTAATCGTGGATCAGAATGCCACG
GTGAATACGTTCCCGGGCCTTGTACACACCGCCCGTCACACCATGGGAGT
GGGTTGCAAAAGAAGTAGGTAGCTTAACCTTCGGGAGGGCGCTTACCACT
TTGTGATTCATGACTGGGGTGAAGTCGTAACAAGGTAACCGTAGGGGAAC
CTGCGGTTGGATCACCTCCTTACCTTAAAGAAGCGTTCTTTG

The match between the T1 sequence and the C1/C2 sequence is

AAAAAATGCGCGGTCAGAAAATTATTTTAAATTTCCTCTTGTCAGGCCGG
AATAACTCCCTATAATGCGCCACCACTGACACGGAACAACGGCAAACACG
CCGCCGGGTCAGCGGGGTTCTCCTGAGAACTCCGGCAGAGAAAGCAAAA
ATAAATGCTTGACTCTGTAGCGGGAA

The match between the T2 sequence and the C1/C2 sequence is

TAAATTTCCTCTTGTCAGGCCGGAATAACTCCCTATAATGCGCCACCACTG
ACACGGAACAACGGCAAACACGCCGCCGGGTCAGCGGGGTTCTCCTGAG
AACTCCGGCAGAGAAAGCAAAAATAAATGCTTGACTCTGTAGCGGGAAG
GCGTATTATGCACACCCCGCGCCGCT

A C1/C2 short loop on chromosome 1 whose identifier is 3432 controls the expression of the genes in this T1/T2 long loop. This C1/C2 short loop is expressed as a RNA single strand that is 3′UTR to the gene btuB and has the DNA sequence

TGCGCGGTCAGAAAATTATTTTAAATTTCCTCTTGTCAGGCCGGAATAACT
CCCTATAATGCGCCACCACTGACACGGAACAACGGCAAACACGCCGCCGG
GTCAGCGGGGTTCTCCTGAGAACTCCGGCAGAGAAAGCAAAAATAAATG
CTTGACTCTGTAGCGGGAAGGCGTATTATGCACACC...ACACCATGGGAGT
GGGTTGCAAAAGAAGTAGGTAGCTTAACCTTCGGGAGGGCGCTTACCACT
TTGTGATTCATGACTGGGGTGAAGTCGTAACAAGGTAACCGTAGGGGAAC
CTGCGGTTGGATCACCTCCTTACCTTAAAGAAGCGT

The match between the T1 sequence and the C1/C2 sequence is

TGCGCGGTCAGAAAATTATTTTAAATTTCCTCTTGTCAGGCCGGAATAACT
CCCTATAATGCGCCACCACTGACACGGAACAACGGCAAACACGCCGCCGG
GTCAGCGGGGTTCTCCTGAGAACTCCGGCAGAGAAAGCAAAAATAAATG
CTTGACTCTGTAGCGGGAA

The match between the T2 sequence and the C1/C2 sequence is

TAAATTTCCTCTTGTCAGGCCGGAATAACTCCCTATAATGCGCCACCACTG
ACACGGAACAACGGCAAACACGCCGCCGGGTCAGCGGGGTTCTCCTGAG
AACTCCGGCAGAGAAAGCAAAAATAAATGCTTGACTCTGTAGCGGGAAG
GCGTATTATGCACACCCCGCGCCGCT

A C1/C2 short loop on chromosome 1 whose identifier is 2218 controls the expression of the genes in this T1/T2 long loop. This C1/C2 short loop is expressed as a RNA single strand that is 3′UTR to the gene clpB and has the DNA sequence

CTTGTCAGGCCGGAATAACTCCCTATAATGCGCCACCACTGACACGGAAC
AACGGCAAACACGCCGCCGGGC

The match between the T1 sequence and the C1/C2 sequence is

CTTGTCAGGCCGGAATAACTCCCTATAATGCGCCACCACTGACACGGAAC
AACGGCAAACACGCCGCCGGGC

The match between the T2 sequence and the C1/C2 sequence is

CTTGTCAGGCCGGAATAACTCCCTATAATGCGCCACCACTGACACGGAAC
AACGGCAAACACGCCGCCGGGC

Example of a Many-to-one Connectron in Archea—M. jannaschii

In this example the existence of the T1-T2 (1630-1643) long loop is controlled by four C1/C2 short loops (1629, 1642, 124 and 1533).

```
                                1629 Chromosome 1
                                1642 Chromosome 1
                                124 Chromosome 1
                                1533 Chromosome 1
                                |
*------------------*------------------*
| Chromosome 1 |
1630 1643
--------------------------------------------------------
```

A double stranded DNA loop of length 4.998 kilo-bases on chromosome 1 is bounded on the left by a T1 sequence whose identifier is 1630. This T1 control element has the DNA sequence

TTATTAATTAGTTCAAAGGATTTTTATTTAATTTCTAAGGGTTTGCTGGTTT
GATTATTTAGAATATTTGAGTTTATTGAATTATTCAGATTTTTAAAAATTA
AGATTAATTAGGAAAGGAAATAAGATTTCTCTAACAGACAAGTTAAATTT
TTGGATTTAAAAAGATAAAAAT

This double stranded DNA loop is bounded on the right by a T2 control element whose identifier is 1643. This T2 control element has the DNA sequence

TTAATTTCTAAGGGTTAGCTGGTTTGATTATTTAGAATATTTGAGTTTATTG
AATTATTCAGATTTTTAAAAATTAGGATTAATTAGGCAAGTAAATAAAAT
TTCTCTAACAAATAAGTTAAATTTTTGGATTTAAAAAGATAAAAATACTCT
GTTTTATTATGGAAAGAAAGAT

This long T1/T2 double stranded DNA loop modulates the expression of the following genes

MJ1597 MJ1598 MJ1599 MJ1600 MJ1601 MJ1602

The expression of genes in this T1/T2 long loop is controlled by the following C1/C2 short loops.

A C1/C2 short loop on chromosome 1 whose identifier is 1629 controls the expression of the genes in this T1/T2 long loop. This C1/C2 short loop is expressed as a RNA single strand that is 3′UTR to the gene MJ1597 and has the DNA sequence

ATATGTTTGAAATTTGAAAATAAGAGTATTTAGAAGTTATTAATTAGTTCA
AAGGATTTTTATTTAATTTCTAAGGGTTTGCTGGTTTGATTATTTAGAATAT
TTGAGTTTATTGAATTATTCAGATTTTTAAAAATTA

The match between the T1 sequence and the C1/C2 sequence is

TTATTAATTAGTTCAAAGGATTTTTATTTAATTTCTAAGGGTTTGCTGGTTT
GATTATTTAGAATATTTGAGTTTATTGAATTATTCAGATTTTTAAAAATTA

The match between the T2 sequence and the C1/C2 sequence is

GCTGGTTTGATTATTTAGAATATTTGAGTTTATTGAATTATTCAGATTTTTA
AAAATTA

A C1/C2 short loop on chromosome 1 whose identifier is 1642 controls the expression of the genes in this T1/T2 long loop. This C1/C2 short loop is expressed as a RNA single strand that is 3 ′UTR to the gene MJ1602 and has the DNA sequence

ATTTAATTTCTAAGGGTTAGCTGGTTTGATTATTTAGAATATTTGAGTTTAT
TGAATTATTCAGATTTTTAAAAATTAGGATTAATTAGGCAAGTAAATAAA
ATTTCTCTAACAAATAAGTTAAATTTTTGGATTTAAAAAGATAAAAATACT
CTGTTTTATTATGGAAAGAAAGAT

The match between the T1 sequence and the C1/C2 sequence is

GCTGGTTTGATTATTTAGAATATTTGAGTTTATTGAATTATTCAGATTTTTA
AAAATTA

The match between the T2 sequence and the C1/C2 sequence is

TTAATTTCTAAGGGTTAGCTGGTTTGATTATTTAGAATATTTGAGTTTATTG
AATTATTCAGATTTTTAAAAATTAGGATTAATTAGGCAAGTAAATAAAAT
TTCTCTAACAAATAAGTTAAATTTTTGGATTTAAAAAGATAAAAATACTCT
GTTTTATTATGGAAAGAAAGAT

A C1/C2 short loop on chromosome 1 whose identifier is 124 controls the expression of the genes in this T1/T2 long loop. This C1/C2 short loop is expressed as a RNA single strand that is 3′UTR to the gene MJ0112 and has the DNA sequence

ATTTAATTTCTAAGGGTTTGCTGGTTTGATTATTTAGAATATTTGAGTTTAT
TGAATTATTCAGATTTTTAAAAT

The match between the T1 sequence and the C1/C2 sequence is

ATTTAATTTCTAAGGGTTTGCTGGTTTGATTATTTAGAATATTTGAGTTTAT
TGAATTATTCAGATTTTTAAAAT

The match between the T2 sequence and the C1/C2 sequence is

GCTGGTTTGATTATTTAGAATATTTGAGTTTATTGAATTATTCAGATTTTTA
AAAAT

A C1/C2 short loop on chromosome 1 whose identifier is 1533 controls the expression of the genes in this T1/T2 long loop. This C1/C2 short loop is expressed as a RNA single strand that is 3′UTR to the gene MJ1486 and has the DNA sequence

TTTTTATTTAATTTCTAAGGGTTTGCTGGTTTGATTATTTAGAATATTTGAG
TTTATT

The match between the T1 sequence and the C1/C2 sequence is

TTTTTATTTAATTTCTAAGGGTTTGCTGGTTTGATTATTTAGAATATTTGAG
TTTATT

The match between the T2 sequence and the C1/C2 sequence is

GCTGGTTTGATTATTTAGAATATTTGAGTTTATT

Example of a Many-to-one Connectron in Single-cell Eukaryotes—S. cervesiae

In this example the existence of the T1-T2 (5515-5533) long loop on chromosome 12 is controlled by seventeen C1/C2 short loops (5516, 5532, 1939, 2323, 1942, 3286, 3649, 4764, 4751, 5536, 6102, 8023, 7356, 3293, 3291, 3289 and 146).

```
                                5516 Chromosome 12
                                5532 Chromosome 12
                                1939 Chromosome 4
                                2323 Chromosome 5
                                1942 Chromosome 5
                                3286 Chromosome 7
                                3649 Chromosome 8
                                4764 Chromosome 12
                                4751 Chromosome 12
                                5536 Chromosome 13
                                6102 Chromosome 14
                                8023 Chromosome 16
                                7356 Chromosome 16
                                3293 Chromosome 8
                                3291 Chromosome 8
                                3289 Chromosome 8
                                146 Chromosome 2
                                |
*------------------*------------------*
| Chromosome 12 |
3197 3308
--------------------------------------------------------
```

A double stranded DNA loop of length 6.466 kilo-bases on chromosome 12 is bounded on the left by a T1 sequence whose identifier is 5515. This T1 control element has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

This double stranded DNA loop is bounded on the right by a T2 control element whose identifier is 5533. This T2 control element has the DNA sequence

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

This long T1/T2 double stranded DNA loop modulates the expression of the following genes

YLR467W

This long T1/T2 double stranded DNA loop modulates the expression of the following C1/C2 short loops

A C1/C2 short loop on chromosome 12 whose identifier is 5516 controls the expression of the genes of one or more other T1/T2 long loops. This C1/C2 short loop is expressed as a RNA single strand that is 3′UTR to the gene YLR464W and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

A C1/C2 short loop on chromosome 12 whose identifier is 5532 controls the expression of the genes of one or more other T1/T2 long loops. This C1/C2 short loop is expressed as a RNA single strand that is 3′UTR to the gene YLR467W and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The expression of genes in this T1/T2 long loop is controlled by the following C1/C2 short loops.

A C1/C2 short loop on chromosome 4 whose identifier is 1939 controls the expression of the genes in this T1/T2 long loop. This C1/C2 short loop is expressed as a RNA single strand that is 3′UTR to the gene YDR545W and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGG

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGG

A C1/C2 short loop on chromosome 5 whose identifier is 2323 controls the expression of the genes in this T1/T2 long loop. This C1/C2 short loop is expressed as a RNA single strand that is 3′UTR to the gene YER189W and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTA
AGAGACAACAGGGCT

A C1/C2 short loop on chromosome 5 whose identifier is 1942 controls the expression of the genes in this T1/T2 long loop. This C1/C2 short loop is expressed as a RNA single strand that is 3′UTR to the gene YEL077C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTA
AGAGACAACAGGGCT

A C1/C2 short loop on chromosome 7 whose identifier is 3286 controls the expression of the genes in this T1/T2 long loop. This C1/C2 short loop is expressed as a RNA single strand that is 3′UTR to the gene YGR296W and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 8 whose identifier is 3649 controls the expression of the genes in this T1/T2 long loop. This C1/C2 short loop is expressed as a RNA single strand that is 3′UTR to the gene YHR219W and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 12 whose identifier is 4764 controls the expression of the genes in this T1/T2 long loop. This C1/C2 short loop is expressed as a RNA single strand that is 3′UTR to the gene YLL066C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 12 whose identifier is 4751 controls the expression of the genes in this T1/T2 long loop. This C1/C2 short loop is expressed as a RNA single strand that is 3′UTR to the gene YLL067C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 13 whose identifier is 5536 controls the expression of the genes in this T1/T2 long loop. This C1/C2 short loop is expressed as a RNA single strand that is 3′UTR to the gene YML133C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 14 whose identifier is 6102 controls the expression of the genes in this T1/T2 long loop. This C1/C2 short loop is expressed as a RNA single strand that is 3′UTR to the gene YNL339C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 16 whose identifier is 8023 controls the expression of the genes in this T1/T2 long loop. This C1/C2 short loop is expressed as a RNA single strand that is 3′UTR to the gene YPR204W and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 16 whose identifier is 7356 controls the expression of the genes in this T1/T2 long loop. This C1/C2 short loop is expressed as a RNA single strand that is 3′UTR to the gene YPL283C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

A C1/C2 short loop on chromosome 8 whose identifier is 3293 controls the expression of the genes in this T1/T2 long loop. This C1/C2 short loop is expressed as a RNA single strand that is 3′UTR to the gene YHL050C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTT

The match between the T2 sequence and the C1/C2 sequence is

ATTATGTATTGTGTAGTATAGTATATTGTAAGAAATTTTTTTTTCTAGGGA
ATATGCGTTTT

A C1/C2 short loop on chromosome 8 whose identifier is 3291 controls the expression of the genes in this T1/T2 long loop. This C1/C2 short loop is expressed as a RNA single strand that is 3′UTR to the gene YHL050C and has the DNA sequence

ATGTAGTAGTATTTCACTGTTTTGATTTAGTGTTTGTTGCACGGCAGTAGC
GAGAGACAAGTGGGAAAGAGTAGGATAAAAAGACAA

The match between the T1 sequence and the C1/C2 sequence is

ATGTAGTAGTATTTCACTGTTTTGATTTAGTGTTTGTTGCACGGCAGTAGC
GAGAGACAAGTGGGAAAGAGTAGGATAAAAAGACAA

The match between the T2 sequence and the C1/C2 sequence is

ATGTAGTAGTATTTCACTGTTTTGATTTAGTGTTTGTTGCACGGCAGTAGC
GAGAGACAAGTGGGAAAGAGTAGGATAAAAAGACAA

A C1/C2 short loop on chromosome 2 whose identifier is 145 controls the expression of the genes in this T1/T2 long loop. This C1/C2 short loop is expressed as a RNA single strand that is 3′UTR to the gene YBL113C and has the DNA sequence

CTATAAAAAGTAAACATAAAATAAAGGTAGTAAGTAGCTTTTGGTTGAAC
ATCCGGGTAAGAGACAACAGGCT

The match between the T1 sequence and the C1/C2 sequence is

CTATAAAAAGTAAACATAAAATAAAGGTAGTAAGTAGCTTTTGGTTG

The match between the T2 sequence and the C1/C2 sequence is

CTATAAAAAGTAAACATAAAATAAAGGTAGTAAGTAGCTTTTGGTTGAAC
ATCCGGGTAAGAGACAACAGGCT

A C1/C2 short loop on chromosome 8 whose identifier is 3289 controls the expression of the genes in this T1/T2 long loop. This C1/C2 short loop is expressed as a RNA single strand that is 3′UTR to the gene YHL050C and has the DNA sequence

CTATAAAAAGTAAACATAAAATAAAGGTAGTAAGTAGCTTTTGGTTGAAC
ATCCGGGTAAGAGACAACAGGCT

The match between the T1 sequence and the C1/C2 sequence is

CTATAAAAAGTAAACATAAAATAAAGGTAGTAAGTAGCTTTTGGTTG

The match between the T2 sequence and the C1/C2 sequence is

CTATAAAAAGTAAACATAAAATAAAGGTAGTAAGTAGCTTTTGGTTGAAC
ATCCGGGTAAGAGACAACAGGCT

A C1/C2 short loop on chromosome 2 whose identifier is 146 controls the expression of the genes in this T1/T2 long loop. This C1/C2 short loop is expressed as a RNA single strand that is 3′UTR to the gene YBL113C and has the DNA sequence

AGGAAATTGTTGTTACGAAAGTCAGTGATTATGTATTGTGTAGTATAGTAT
ATTGTAAGAAA

The match between the T1 sequence and the C1/C2 sequence is

AGGAAATTGTTGTTACGAAAGTCAGTGATTATGTATTGTGTAGTATAGTAT
ATTGTAAGAAA

The match between the T2 sequence and the C1/C2 sequence is

ATTATGTATTGTGTAGTATAGTATATTGTAAGAAA

Example of a Many-to-one Connectron in Multi-cell Eukaryotes—C. elegans

In this example the existence of the T1-T2 (3197-3308) long loop on chromosome 5 is controlled by three C1/C2 short loops (4382, 4375 and 28633).

```
                                  4382 Chromosome 1
                                  4375 Chromosome 1
                                  28633 Chromosome 5
                                  |
*-------------------*-------------------*
| Chromosome 5 |
28632 28697
--------------------------------------------------------
```

A double stranded DNA loop of length 58.451 kilo-bases on chromosome 5 is bounded on the left by a T1 sequence whose identifier is 28632. This T1 control element has the DNA sequence

GCAAAAATTGACTGAAAATTTGAATTTCCCGCAAAAAATTGACTGAAAAT
TTGAATTTCCCGCCAAAAATTGACTGAAAATTTGAA

This double stranded DNA loop is bounded on the right by a T2 control element whose identifier is 28697. This T2 control element has the DNA sequence

CAAAAAATTGACTGAAAATTTGAATTTCCCTCCAAAAATTGACTGAAAAT
TTGAATTTCCCGCCAAAAATTGACTGAAAATTTGAATATCCCGCCAAAAA
TTGACTGAAAATTTGAATTTCCCGCCGAAAATTAAATGAAAAATGGAATT
TCTCGCCGAA

This long T1/T2 double stranded DNA loop modulates the expression of the following genes

M162.8 M162.4 M162.3 M162.6 M162.2 M162.1 M162.7

The expression of genes in this T1/T2 long loop is controlled by the following C1/C2 short loops.

A C1/C2 short loop on chromosome 1 whose identifier is 4382 controls the expression of the genes in this T1/T2 long loop. This C1/C2 short loop is expressed as a RNA single strand that is 3′UTR to the gene Y43F8B.10 and has the DNA sequence

ATTATAGAAAATTTAAATTTCCCTCCAAAAAATTGACTGAAAATTTGAATT
TCCCTCCAAAAATTGACTGAAAATTTGAATTTCCCGCCAAAAATTGACTG
AAAATTTGAATATCCCGCCAAAAATTGACTGAAAATTTGAATTTCCCGCC
GAAAATTAAATGAAAAATGGAATTTCTCGCCGAAAAATTCAGTAAAAATT
TGAATTTCCTGCCAAAAATTGACTGAAAATTTGAATTTCTTGCCAAAAAA
GTGACTGGGAATTTGAATTTCCCTCCAAAAATTGACTGAAATTTTGAATTT
CCCGCTAAAAGTTGACT

The match between the T1 sequence and the C1/C2 sequence is

CAAAAATTGACTGAAAATTTGAATTTCCCGC

The match between the T2 sequence and the C1/C2 sequence is

CAAAAAATTGACTGAAAATTTGAATTTCCCTCCAAAAATTGACTGAAAAT
TTGAATTTCCCGCCAAAAATTGACTGAAAATTTGAATATCCCGCCAAAAA
TTGACTGAAAATTTGAATTTCCCGCCGAAAATTAAATGAAAAATGGAATT
TCTCGCCGAA

A C1/C2 short loop on chromosome 1 whose identifier is 4375 controls the expression of the genes in this T1/T2 long loop. This C1/C2 short loop is expressed as a RNA single strand that is 3′UTR to the gene Y43F8B.10 and has the DNA sequence

ATTATAGAAAATTTAAATTTCCCTCCAAAAAATTGACTGAAAATTTGAATT
TCCCTCCAAAAATTGACTGAAAATTTGAATTTCCCGCCAAAAATTGACTG
AAAATTTGAATATCCCGCCAAAAATTGACTGAAAATTTGAATTTCCCGCC
GAAAATTAAATGAAAAATGGAATTTCTCGCCGAAAAATTCAGTAAAAATT
TGAATTTCCTGCCAAAATTGACTGAAAATTTGAATTTCTTGCCAAAAAA
GTGACTGGGAATTTGAATTTCCCTCCAAAAATTGACTGAAATTTTGAATTT
CCCGCTAAAAGTTGACT

The match between the T1 sequence and the C1/C2 sequence is

CAAAAATTGACTGAAAATTTGAATTTCCCGC

The match between the T2 sequence and the C1/C2 sequence is

CAAAAAATTGACTGAAAATTTGAATTTCCCTCCAAAAATTGACTGAAAAT
TTGAATTTCCCGCCAAAAATTGACTGAAAATTTGAATATCCCGCCAAAAA
TTGACTGAAAATTTGAATTTCCCGCCGAAAATTAAATGAAAAATGGAATT
TCTCGCCGAA

A C1/C2 short loop on chromosome 5 whose identifier is 28633 controls the expression of the genes in this T1/T2 long loop. This C1/C2 short loop is expressed as a RNA single strand that is 3′UTR to the gene M162.5 and has the DNA sequence

CAAAAATTGACTGAAAATTTGAATTTCCCGCAAAAAATTGACTGAAAATT
TGAATTTCCCGCCAAAAATTGACTGAAAATTTGAA

The match between the T1 sequence and the C1/C2 sequence is

CAAAAATTGACTGAAAATTTGAATTTCCCGCAAAAAATTGACTGAAAATT
TGAATTTCCCGCCAAAAATTGACTGAAAATTTGAA

The match between the T2 sequence and the C1/C2 sequence is

CAAAAAATTGACTGAAAATTTGAATTTCCC

3. One Connectron Controls the Expression of Many Sets of Genes in Prokaryotes, Archea, Single-celled Eukaryotes and Multi-celled Eukaryotes.

One C1/C2 short loop can control the existence of a many T1-T2 long loops. The C1/C2 short loop can be on the same chromosome or on different chromosomes from the T1-T2 long loops. This relationship is described as “one-to-many”. This relationship exists in prokaryotes, archea, single-celled eukaryotes and multi-celled eukaryotes.

Example of a One-to-many Connectron in Prokaryotes—E. coli

In this example the existence of T1-T2 (3208-3315, 3436-3476, 3439-3478 and 3441-3479) long loops are controlled by one C1/C2 short loop (3206).

```
                                3206 Chromosome 1
                                |
*------------------*------------------*
| Chromosome 1 |
3208 3315
                                3206 Chromosome 1
                                |
*------------------*------------------*
| Chromosome 1 |
3436 3476
                                3206 Chromosome 1
                                |
*------------------*------------------*
| Chromosome 1 |
3439 3478
                                3206 Chromosome 1
                                |
*------------------*------------------*
| Chromosome 1 |
3441 3479
--------------------------------------------------------
```

A double stranded DNA loop of length 93.377 kilo-bases on chromosome 1 is bounded on the left by a T1 sequence whose identifier is 3208. This T1 control element has the DNA sequence

ACTCATCTTCGGGTGATGTTTGAGATATTTGCTCTTTAAAAATCTGGATCA
AGCTGAAAATTGAAACACTGAACAACGAAAGTTGTTCGTGAGTCTCTCAA
ATTTTCGCAACACGATGATGAATCGAAAGAAACATCTTCGGGTTGTGAGG
TTAAGCGACTAAGCGTACACGGTGGATGCCCTGGC...AGTGTGTTTCGACA
CACTATCATTAACTGAATCCATAGGTTAATGAGGCGAACCGGGGGAACTG
AAACATCTAAGTACCCCGAGGAAAAGAAATCAACCGAGATTCCCCCAGTA
GCGGCGAGCGAACGGGGAGCAGCCCAGAGCCTGAATCAGT

This double stranded DNA loop is bounded on the right by a T2 control element whose identifier is 3315. This T2 control element has the DNA sequence

TTTGCTCTTTAAAAATCTGGATCAAGCTGAAAATTGAAACACTGAACAAC
GAAAGTTGTTCGTGAGTCTCTCAAATTTTCGCAACTCTGAAGTGAAACATC
TTCGGGTTGTGAGGTTAAGCGACTAAGCGTACACGGTGGATGCCCTGGCA
GTCAGAGGCGATGAAGGACGTGCTAATCTGCGATA...GGTTAATGAGGCG
AACCGGGGGAACTGAAACATCTAAGTACCCCGAGGAAAAGAAATCAACC
GAGATTCCCCCAGTAGCGGCGAGCGAACGGGGAGCAGCCCAGAGCCTGA
ATCAGTGTGTGTGTTAGTGGAAGCGTCTGGAAA

This long T1/T2 double stranded DNA loop modulates the expression of the following genes

| | | | | | | | | |
|---|---|---|---|---|---|---|---|---|
| rrlC | rrfC | aspT | trpT | yifA | yifE | yifB | ilvL | ilvG_1 |
| ilvM | ilvE | ilvD | ilvA | ilvY | ilvC | ppiC | b3776 | rep |
| gppA | rhlB | trxA | rhoL | rho | rfe | wzzE | wecB | rffH |
| wecD | wecE | wzxE | yifM_2 | wecG | yifK | argX | hisR | |
| leuT | proM | aslB | aslA | hemY | hemX | hemD | cyaA | |
| cyaY | b3808 | dapF | uvrD | b3814 | corA | yigF | yigG | rarD |
| yigI | pldA | recQ | yigJ | yigK | pldB | yigL | yigM | metR |
| metE | ysgA | udp | yigN | ubiE | yigP | b3836 | yigU | |
| yigW_1 | rfaH | yigC | ubiB | fadA | fadB | pepQ | trkH | |
| hemG | rrsA | ileT | | | | | | |

The expression of genes in this T1/T2 long loop is controlled by the following C1/C2 short loops.

A C1/C2 short loop on chromosome 1 whose identifier is 3206 controls the expression of the genes in this T1/T2 long loop. This C1/C2 short loop is expressed as a RNA single strand that is 3′UTR to the gene rrsC and has the DNA sequence

GTCCCCTTCGTCTAGAGGCCCAGGACACCGCCCTTTCACGGCGGTAACAG
GGGTTCGAATCCCCTAGGGGACGCCACTTGCTGGTTTGTGAGTGAAAGTC
ACCTGCCTTAATATCTCAAAACTCATCTTCGGGTGATGTTTGAGATATTTG
CTCTTTAAAAATCTGGATCAAGCTGAAAATTGAAA...ACCGGCGATTTCCG
AATGGGGAAACCCAGTGTGTTTCGACACACTATCATTTAACTGAATCCATA
GGTTAATGAGGCGAACCGGGGGAACTGAAACATCTAAGTACCCCGAGGA
AAAGAAATCAACCGAGATTCCCCCAGTAGCGGCGAGCGAACGGGGAGCA
GCCCAGAGCCTGAATCAGT

The match between the T1 sequence and the C1/C2 sequence is

ACTCATCTTCGGGTGATGTTTGAGATATTTGCTCTTTAAAAATCTGGATCA
AGCTGAAAATTGAAACACTGAACAACGAAAGTTGTTCGTGAGTCTCTCAA
ATTTTCGCAACACGATGATGAATCGAAAGAAACATCTTCGGGTTGTGAGG
TTAAGCGACTAAGCGTACACGGTGGATGCCCTGGC...AGTGTGTTTCGACA
CACTATCATTAACTGAATCCATAGGTTAATGAGGCGAACCGGGGGAACTG
AAACATCTAAGTACCCCGAGGAAAAGAAATCAACCGAGATTCCCCCAGTA
GCGGCGAGCGAACGGGGAGCAGCCCAGAGCCTGAATCAGT

The match between the T2 sequence and the C1/C2 sequence is

TTTGCTCTTTAAAAATCTGGATCAAGCTGAAAATTGAAACACTGAACAAC
GAAAGTTGTTCGTGAGTCTCTCAAATTTTCGCAAC

A double stranded DNA loop of length 41.279 kilo-bases on chromosome 1 is bounded on the left by a T1 sequence whose identifier is 3436. This T1 control element has the DNA sequence

ACGCAACGCGTGATAAGCAATTTTCGTGTCCCCTTCGTCTAGAGGCCCAG
GACACCGCCCTTTCACGGCGGTAACAGGGGTTCGAATCCCCTAGGGGACG
CCACTTGCTGGTT

This double stranded DNA loop is bounded on the right by a T2 control element whose identifier is 3476. This T2 control element has the DNA sequence

AGTGAAAAGCAAGGCGTCTTGCGAAGCAGACTGATACGTCCCCTTCGTCT
AGAGGCCCAGGACACCGCCCTTTCACGGCGGTAACAGGGGTTCGAATCCC
CTAGGGGACGCCACTTGCTGGTTTGTGAGTGAAAGTCACCTGCCTTAATA

This long T1/T2 double stranded DNA loop modulates the expression of the following genes

| | | | | | | | | |
|---|---|---|---|---|---|---|---|---|
| gltT | rrlB | rrfB | murB | coaA | b3975 | tyrU | thrT | tufB |
| secE | nusG | rplK | rplA | rplJ | rplL | rpoB | rpoC | htrC |
| thiH | thiF | thiE | yjaE | yjaD | hemE | nfi | yjaG | hupA |
| yjaH | yjaI | hydH | purD | purH | | | | |

This long T1/T2 double stranded DNA loop modulates the expression of the following C1/C2 short loops

A C1/C2 short loop on chromosome 1 whose identifier is 3206 controls the expression of the genes in this T1/T2 long loop. This C1/C2 short loop is expressed as a RNA single strand that is 3′UTR to the gene rrsC and has the DNA sequence

GTCCCCTTCGTCTAGAGGCCCAGGACACCGCCCTTTCACGGCGGTAACAG
GGGTTCGAATCCCCTAGGGGACGCCACTTGCTGGTTTGTGAGTGAAAGTC
ACCTGCCTTAATATCTCAAAACTCATCTTCGGGTGATGTTTGAGATATTTG
CTCTTTAAAAATCTGGATCAAGCTGAAAATTGAAACACTGAACAACGAAA
GTTGTTCGTGAGTCTCTCAAATTTTCGCAACACGATGATGAATCGAAAGA
AACATCTTCGGGTTGTGAGGTTAAGCGACTAAGCGTACACGGTGGATGCC
CTGGCAGTCAGAGGCGATGAAGGACGTGCTAATCTGCGATAAGCGTCGGT
AAGGTGATATGAACCGTTATAACCGGCGATTTCCGAATGGGGAAACCCAG
TGTGTTTCGACACACTATCATTAACTGAATCCATAGGTTAATGAGGCGAA
CCGGGGGAACTGAAACATCTAAGTACCCCGAGGAAAAGAAATCAACCGA
GATTCCCCCAGTAGCGGCGAGCGAACGGGGAGCAGCCCAGAGCCTGAAT
CAGT

The match between the T1 sequence and the C1/C2 sequence is

GTCCCCTTCGTCTAGAGGCCCAGGACACCGCCCTTTCACGGCGGTAACAG
GGGTTCGAATCCCCTAGGGGACGCCACTTGCTGGTT

The match between the T2 sequence and the C1/C2 sequence is

GTCCCCTTCGTCTAGAGGCCCAGGACACCGCCCTTTCACGGCGGTAACAG
GGGTTCGAATCCCCTAGGGGACGCCACTTGCTGGTTTGTGAGTGAAAGTC
ACCTGCCTTAATA

A double stranded DNA loop of length 41.336 kilo-bases on chromosome 1 is bounded on the left by a T1 sequence whose identifier is 3439. This T1 control element has the DNA sequence

CCTTAATATCTCAAAACTCATCTTCGGGTGATGTTTGAGATATTTGCTCTTT
AAAAATCTGGATCAAGCTGAAAATTGAAACACTGAACAACGA

This double stranded DNA loop is bounded on the right by a T2 control element whose identifier is 3478. This T2 control element has the DNA sequence

GTGATGTTTGAGATATTTGCTCTTTAAAAATCTGGATCAAGCTGAAAATTG
AAACACTGAACAACGAAAGTTGTTCGTGAGTCTCTCAAATTTT

This long T1/T2 double stranded DNA loop modulates the expression of the following genes

| | | | | | | | | |
|---|---|---|---|---|---|---|---|---|
| rrlB | rrfB | murB | coaA | b3975 | tyrU | thrT | tufB | secE |
| nusG | rplK | rplA | rplJ | rplL | rpoB | rpoC | htrC | thiH |
| thiF | thiE | yjaE | yjaD | hemE | nfi | yjaG | hupA | yjaH |
| yjaI | hydH | purD | purH | gltV | | | | |

The expression of genes in this T1/T2 long loop is controlled by the following C1/C2 short loops.

A C1/C2 short loop on chromosome 1 whose identifier is 3206 controls the expression of the genes in this T1/T2 long loop. This C1/C2 short loop is expressed as a RNA single strand that is 3′UTR to the generrsC and has the DNA sequence

GTCCCCTTCGTCTAGAGGCCCAGGACACCGCCCTTTCACGGCGGTAACAG
GGGTTCGAATCCCCTAGGGGACGCCACTTGCTGGTTTGTGAGTGAAAGTC
ACCTGCCTTAATATCTCAAAACTCATCTTCGGGTGATGTTTGAGATATTTG
CTCTTTAAAAATCTGGATCAAGCTGAAAATTGAAA...ACCGGCGATTTCCG
AATGGGGAAACCCAGTGTGTTTCGACACACTATCATTAACTGAATCCATA
GGTTAATGAGGCGAACCGGGGGAACTGAAACATCTAAGTACCCCGAGGA
AAAGAAATCAACCGAGATTCCCCCAGTAGCGGCGAGCGAACGGGGAGCA
GCCCAGAGCCTGAATCAGT

The match between the T1 sequence and the C1/C2 sequence is

CCTTAATATCTCAAAACTCATCTTCGGGTGATGTTTGAGATATTTGCTCTTT
AAAAATCTGGATCAAGCTGAAAATTGAAACACTGAACAACGA

The match between the T2 sequence and the C1/C2 sequence is

GTGATGTTTGAGATATTTGCTCTTTAAAAATCTGGATCAAGCTGAAAATTG
AAACACTGAACAACGAAAGTTGTTCGTGAGTCTCTCAAATTTT

A double stranded DNA loop of length 38.285 kilo-bases on chromosome 1 is bounded on the left by a T1 sequence whose identifier is 3441. This T1 control element has the DNA sequence

AATTTTCGCAACACGATGATGAATCGAAAGAAACATCTTCGGGTTGTGAG
GTTAAGCGACTAAGCGTACACGGTGGATGCCCTGGCAGTCAGAGGCGATG
AAGGACGTGCTAATCTGCGATAAGCGTCGGTAAGGTGATATGAACCGTTA
TAACCGGCGATTTCCGAATGGGGAAACCCAGTGTGT...GATGAGAGAAGA
TTTTCAGCCTGATACAGATTAAATCAGAACGCAGAAGCGGTCTGATAAAA
CAGAATTTGCCTGGCGGCAGTAGCGCGGTGGTCCCACCTGACCCCATGCC
GAACTCAGAAGTGAAACGCCGTAGCGCCGATGGTAGTGTGGGGTCTCCCC
ATGCGAG

This double stranded DNA loop is bounded on the right by a T2 control element whose identifier is 3479. This T2 control element has the DNA sequence

AAGAAACATCTTCGGGTTGTGAGGTTAAGCGACTAAGCGTACACGGTGGA
TGCCCTGGCAGTCAGAGGCGATGAAGGACGTGCTAATCTGCGATAAGCGT
CGGTAAGGTGATATGAACCGTTATAACCGGCGATTTCCGAATGGGGAAAC
CCAGTGTGTTTCGACACACTATCATTAACTGAATCC...CAGATTAAATCAG
AACGCAGAAGCGGTCTGATAAAACAGAATTTGCCTGGCGGCAGTAGCGC
GGTGGTCCCACCTGACCCCATGCCGAACTCAGAAGTGAAACGCCGTAGCG
CCGATGGTAGTGTGGGGTCTCCCCATGCGAGAGTAGGGAACTGCCAGGCA
TCAAATTA

This long T1/T2 double stranded DNA loop modulates the expression of the following genes

| | | | | | | | | |
|---|---|---|---|---|---|---|---|---|
| rrlB | rrfB | murB | coaA | b3975 | tyrU | thrT | tufB | secE |
| nusG | rplK | rplA | rplJ | rplL | rpoB | rpoC | htrC | thiH |
| thiF | thiE | yjaE | yjaD | hemE | nfi | yjaG | hupA | yjaH |
| yjaI | hydH | purD | purH | gltV | | | | |

The expression of genes in this T1/T2 long loop is controlled by the following C1/C2 short loops.

A C1/C2 short loop on chromosome 1 whose identifier is 3206controls the expression of the genes in this T1/T2 long loop. This C1/C2 short loop is expressed as a RNA single strand that is 3′UTR to the gene rrsC and has the DNA sequence

GTCCCCTTCGTCTAGAGGCCCAGGACACCGCCCTTTCACGGCGGTAACAG
GGGTTCGAATCCCCTAGGGGACGCCACTTGCTGGTTTGTGAGTGAAAGTC
ACCTGCCTTAATATCTCAAAACTCATCTTCGGGTGATGTTTGAGATATTTG
CTCTTTAAAAATCTGGATCAAGCTGAAAATTGAAA...ACCGGCGATTTCCG
AATGGGGAAACCCAGTGTGTTTCGACACACTATCATTAACTGAATCCATA
GGTTAATGAGGCGAACCGGGGGAACTGAAACATCTAAGTACCCCGAGGA
AAAGAAATCAACCGAGATTCCCCCAGTAGCGGCGAGCGAACGGGGAGCA
GCCCAGAGCCTGAATCAGT

The match between the T1 sequence and the C1/C2 sequence is

AATTTTCGCAACACGATGATGAATCGAAAGAAACATCTTCGGGTTGTGAG
GTTAAGCGACTAAGCGTACACGGTGGATGCCCTGGCAGTCAGAGGCGATG
AAGGACGTGCTAATCTGCGATAAGCGTCGGTAAGGTGATATGAACCGTTA
TAACCGGCGATTTCCGAATGGGGAAACCCAGTGTGTTTCGACACACTATC
ATTAACTGAATCCATAGGTTAATGAGGCGAACCGGGGGAACTGAAACATC
TAAGTACCCCGAGGAAAAGAAATCAACCGAGATTCCCCCAGTAGCGGCG
AGCGAACGGGGAGCAGCCCAGAGCCTGAATCAGT

The match between the T2 sequence and the C1/C2 sequence is

AAGAAACATCTTCGGGTTGTGAGGTTAAGCGACTAAGCGTACACGGTGGA
TGCCCTGGCAGTCAGAGGCGATGAAGGACGTGCTAATCTGCGATAAGCGT
CGGTAAGGTGATATGAACCGTTATAACCGGCGATTTCCGAATGGGGAAAC
CCAGTGTGTTTCGACACACTATCATTAACTGAATCCATAGGTTAATGAGGC
GAACCGGGGGAACTGAAACATCTAAGTACCCCGAGGAAAAGAAATCAAC
CGAGATTCCCCCAGTAGCGGCGAGCGAACGGGGAGCAGCCCAGAGCCTG
AATCAGT

Example of a One-to-many Connectron in Archea—M. jannaschii

In this example the existence of T1-T2 (534-611, 1139-1159, and 1630-1643) long loops are controlled by one C1/C2 short loop (1642).

```
1642 Chromosome 1
                          |
*------------------*------------------*
| Chromosome 1  |
534  611
                            1642 Chromosome 1
                            |
*------------------*------------------*
| Chromosome 1  |
1139  1159
                          1642 Chromosome 1
                          |
*------------------*------------------*
| Chromosome 1  |
1630  1643
-------------------------------------------------------
```

A double stranded DNA loop of length 72.886 kilo-bases on chromosome 1 is bounded on the left by a T1 sequence whose identifier is 534. This T1 control element has the DNA sequence

TAAGTAAATAAAATTTCTCTAACAAATAAGTTAAATT

This double stranded DNA loop is bounded on the right by a T2 control element whose identifier is 611. This T2 control element has the DNA sequence

TAAATAAAATTTCTCTAACAAATAAGTTAAATTTTTGGATTTAAAAAGATA
AAAATGCT

This long T1/T2 double stranded DNA loop modulates the expression of the following genes

| | | | | | | |
|---|---|---|---|---|---|---|
| MJ0486 | MJ0487 | MJ0488 | MJ0489 | MJ0490 | MJ0492 | MJ0493 |
| MJ0494 | MJ0495 | MJ0496 | MJ0497 | MJ0499 | MJ0500 | MJ0501 |
| MJ0502 | MJ0503 | MJ0504 | MJ0506 | MJ0507 | MJ0508 | MJ0509 |
| MJ0510 | MJ0511 | MJ0512 | MJ0513 | MJ0514 | MJ0514 | MJ0517 |
| MJ0519 | MJ0520 | MJ0521 | MJ0522 | MJ0523 | MJ0525 | MJ0526 |
| MJ0526 | MJ0529 | MJ0530 | MJ0531 | MJ0532 | MJ0534 | MJ0535 |
| MJ0536 | MJ0538 | MJ0539 | MJ0540 | MJ0541 | MJ0542 | MJ0543 |
| MJ0544 | MJ0545 | MJ0547 | MJ0548 | MJ0549 | MJ0550 | MJ0552 |
| MJ0553 | MJ0554 | MJ0555 | MJ0556 | MJ0558 | MJ0559 | MJ0560 |
| MJ0561 | MJ0562 | MJ0563 | MJ0564 | | | |

The expression of genes in this T1/T2 long loop is controlled by the following C1/C2 short loops.

A C1/C2 short loop on chromosome 1 whose identifier is 1642 controls the expression of the genes in this T1/T2 long loop. This C1/C2 short loop is expressed as a RNA single strand that is 3′UTR to the gene MJ1602 and has the DNA sequence

ATTTAATTTCTAAGGGTTAGCTGGTTTGATTATTTAGAATATTTGAGTTTAT
TGAATTATTCAGATTTTTAAAAATTAGGATTAATTAGGCAAGTAAATAAA
ATTTCTCTAACAAATAAGTTAAATTTTTGGATTTAAAAAGATAAAAATACT
CTGTTTTATTATGGAAAGAAAGAT

The match between the T1 sequence and the C1/C2 sequence is

AAGTAAATAAAATTTCTCTAACAAATAAGTTAAATT

The match between the T2 sequence and the C1/C2 sequence is

TAAATAAAATTTCTCTAACAAATAAGTTAAATTTTTGGATTTAAAAAGATA
AAAAT

A double stranded DNA loop of length 14.509 kilo-bases on chromosome 1 is bounded on the left by a T1 sequence whose identifier is 1139. This T1 control element has the DNA sequence

ATTTATTAATTAGTTCAAAGGATTTTTATTTAATTTCTAAGGGTTAGCTGG
TTTGATTGTTTAAAATATTTGAGTTTA

This double stranded DNA loop is bounded on the right by a T2 control element whose identifier is 1159. This T2 control element has the DNA sequence

ATTTAATTTCTAAGGGTTAGCTGGTTTGATTATTTAGAATATTTGAGTTTAT
TGAATTATTCAGATTTTTAAAAATTA

This long T1/T2 double stranded DNA loop modulates the expression of the following genes

| | | | | | | |
|---|---|---|---|---|---|---|
| MJ1096 | MJ1097 | tRNA-Arg-3 | MJ1098 | MJ1099 | MJ1100 | MJ1101 |
| MJ1102 | MJ1103 | MJ1104 | MJ1105 | MJ1106 | MJ1107 | MJ1108 |

The expression of genes in this T1/T2 long loop is controlled by the following C1/C2 short loops.

A C1/C2 short loop on chromosome 1 whose identifier is 1642 controls the expression of the genes in this T1/T2 long loop. This C1/C2 short loop is expressed as a RNA single strand that is 3′UTR to the gene MJ1602 and has the DNA sequence

ATTTAATTTCTAAGGGTTAGCTGGTTTGATTATTTAGAATATTTGAGTTTAT
TGAATTATTCAGATTTTTAAAAATTAGGATTAATTAGGCAAGTAAATAAA
ATTTCTCTAACAAATAAGTTAAATTTTTGGATTTAAAAAGATAAAAATACT
CTGTTTTATTATGGAAAGAAAGAT

The match between the T1 sequence and the C1/C2 sequence is

ATTTAATTTCTAAGGGTTAGCTGGTTTGATT

The match between the T2 sequence and the C1/C2 sequence is

ATTTAATTTCTAAGGGTTAGCTGGTTTGATTATTTAGAATATTTGAGTTTAT
TGAATTATTCAGATTTTTAAAAATTA

A double stranded DNA loop of length 4.998 kilo-bases on chromosome 1 is bounded on the left by a T1 sequence whose identifier is 1630. This T1 control element has the DNA sequence

TTATTAATTAGTTCAAAGGATTTTTATTTAATTTCTAAGGGTTTGCTGGTTT
GATTATTTAGAATATTTGAGTTTATTGAATTATTCAGATTTTTAAAAATTA
AGATTAATTAGGAAAGGAAATAAGATTTCTCTAACAGACAAGTTAAATTT
TTGGATTTAAAAAGATAAAAAT

This double stranded DNA loop is bounded on the right by a T2 control element whose identifier is 1643. This T2 control element has the DNA sequence

TTAATTTCTAAGGGTTAGCTGGTTTGATTATTTAGAATATTTGAGTTTATTG
AATTATTCAGATTTTTAAAAATTAGGATTAATTAGGCAAGTAAATAAAAT
TTCTCTAACAAATAAGTTAAATTTTTGGATTTAAAAAGATAAAAATACTCT
GTTTTATTATGGAAAGAAAGAT

This long T1/T2 double stranded DNA loop modulates the expression of the following genes

MJ1597 MJ1598 MJ1599 MJ1600 MJ1601 MJ1602

The expression of genes in this T1/T2 long loop is controlled by the following C1/C2 short loops.

A C1/C2 short loop on chromosome 1 whose identifier is 1642 controls the expression of the genes in this T1/T2 long loop. This C1/C2 short loop is expressed as a RNA single strand that is 3′UTR to the gene MJ1602 and has the DNA sequence

ATTTAATTTCTAAGGGTTAGCTGGTTTGATTATTTAGAATATTTGAGTTTAT
TGAATTATTCAGATTTTTAAAAATTAGGATTAATTAGGCAAGTAAATAAA
ATTTCTCTAACAAATAAGTTAAATTTTTGGATTTAAAAAGATAAAAATACT
CTGTTTTATTATGGAAAGAAAGAT

The match between the T1 sequence and the C1/C2 sequence is

GCTGGTTTGATTATTTAGAATATTTGAGTTTATTGAATTATTCAGATTTTTA
AAAATTA

The match between the T2 sequence and the C1/C2 sequence is

TTAATTTCTAAGGGTTAGCTGGTTTGATTATTTAGAATATTTGAGTTTATTG
AATTATTCAGATTTTTAAAAATTAGGATTAATTAGGCAAGTAAATAAAAT
TTCTCTAACAAATAAGTTAAATTTTTGGATTTAAAAAGATAAAAATACTCT
GTTTTATTATGGAAAGAAAGAT

Example of a One-to-many Connectron in Single-cell Eukaryotes—S. cervesiae

In this example the existence of T1-T2 (158-171, 293-317, 4295-4308 and 5916-5923) long loops are controlled by one C1/C2 short loop (86).

```
86 Chromosome 1
                          |
*------------------*------------------*
| Chromosome 1  |
158  171
                          86 Chromosome 1
                          |
*------------------*-------------------*
| Chromosome 1  |
293  317
                          86 Chromosome 1
                          |
*-------------------*------------------*
| Chromosome 10  |
4295  4308
                          86 Chromosome 1
                          |
*-------------------*------------------*
| Chromosome 13  |
5916  5923
---------------------------------------------------------
```

A double stranded DNA loop of length 20.391 kilo-bases on chromosome 2 is bounded on the left by a T1 sequence whose identifier is 158. This T1 control element has the DNA sequence

CCAATTGTTGGAATAAAAATCAACTATCATCTACTAACTAGTATTTACGTT
ACTAGTATATTATCATATACGGTGTTAGAAGATGACGCAAATGATGAGAA
ATAGTCATCTAAATTAGTGGAAGCTGAAACGCAAGGATTGATAATGTAAT
AG

This double stranded DNA loop is bounded on the right by a T2 control element whose identifier is 171. This T2 control element has the DNA sequence

ATAATTGTTGGAATAAAAATCAACTATCATCTACTAACTAGTATTTACGTT
ACTAGTATATTATCATATACGGTGTTAGAAGATGACACAAATGATGAGAA
ATAGTCATCTAAATTAGTGGAAGCTGAAACGCAAGGATTGATAATGTAAT
AGGATCAATGAATATTAACATATAAAATGATGATAATAATA

This long T1/T2 double stranded DNA loop modulates the expression of the following genes

| | | | | | |
|---|---|---|---|---|---|
| YBL107W-A | TL(UAA)B1 | YBL107C | YBL106C | YBL105C | YBL104C |
| YBL103C | YBL102W | YBL101C | | | |

The expression of genes in this T1/T2 long loop is controlled by the following C1/C2 short loops.

A C1/C2 short loop on chromosome 1 whose identifier is 86 controls the expression of the genes in this T1/T2 long loop. This C1/C2 short loop is expressed as a RNA single strand that is 3′UTR to the gene YAR009C and has the DNA sequence

ATCTATTACATTATGGGTGGTATGTTGGAATAGAAATCAACTATCATCTAC
TAACTAGTATTTACATTACTAGTATATTATCATATACGGTGTTAGAAGATG
ACGCAAATGATGAGAAATAGTCATCTAAATTAGTGGAAGCTGAAACGCA
AGGATTGATAATGTAATAGGATCAATGAATATAAACATATAAAACGGAAT
GAGGAATAATCGTAATATTAGTATGTAGAAATATAGATTCCATTTTGAGG
ATTCCTATATCCTCGAGGAGAACTTCTAGTATATTCTGTATACCTAATATT
ATAGCCTTTATCAACAATGGAATCCCAACAATTATCTCAACATTCACCCAT
TTCTCAGAA

The match between the T1 sequence and the C1/C2 sequence is

AAATCAACTATCATCTACTAACTAGTATTTAC

The match between the T2 sequence and the C1/C2 sequence is

AAATCAACTATCATCTACTAACTAGTATTTAC

A double stranded DNA loop of length 38.470 kilo-bases on chromosome 2 is bounded on the left by a T1 sequence whose identifier is 293. This T1 control element has the DNA sequence

GAATTGTTGGAATAAAAATCCACTATCGTCTATCAACTAATAGTTATATTA
TCAATATATTATCATATACGGTGTTAAGATGATGACATAAGTTATGAGAA
GCTGTCATCGAAGTTAGAGGAAGCTGAAGTGCAAGGATTGATAATGTAAT
AGGATAATGAAACATATAAAACGGAATGAGGAATAATCGTAATATTAGT
ATGTAGAAATATAGATTCCATTTTGAGGATTCCTATATCCTTGAGGAGAAC
TTCTAGT

This double stranded DNA loop is bounded on the right by a T2 control element whose identifier is 317. This T2 control element has the DNA sequence

AATATTAGTATGTAGAAATATAGATTCCATTTTGAGGATTCCTATATCCTC
GAGGAGAACTTCTAGTATATTCTGTA

This long T1/T2 double stranded DNA loop modulates the expression of the following genes

| | | | | | |
|---|---|---|---|---|---|
| YBL005W-B | TS(AGA)B | YBL004W | YBL003C | YBL002W | YBL001C |
| YBR001C | YBR002C | YBR003W | YBR004C | YBR005W | YBR006W |
| YBR007C | YBR008C | YBR009C | YBR010W | YBR011C | YBR012C |

The expression of genes in this T1/T2 long loop is controlled by the following C1/C2 short loops.

A C1/C2 short loop on chromosome 1 whose identifier is 86 controls the expression of the genes in this T1/T2 long loop. This C1/C2 short loop is expressed as a RNA single strand that is 3′UTR to the gene YAR009C and has the DNA sequence

ATCTATTACATTATGGGTGGTATGTTGGAATAGAAATCAACTATCATCTAC
TAACTAGTATTTACATTACTAGTATATTATCATATACGGTGTTAGAAGATG
ACGCAAATGATGAGAAATAGTCATCTAAATTAGTGGAAGCTGAAACGCA
AGGATTGATAATGTAATAGGATCAATGAATATAAACATATAAAACGGAAT
GAGGAATAATCGTAATATTAGTATGTAGAAATATAGATTCCATTTTGAGG
ATTCCTATATCCTCGAGGAGAACTTCTAGTATATTCTGTATACCTAATATT
ATAGCCTTTATCAACAATGGAATCCCAACAATTATCTCAACATTCACCCAT
TTCTCAGAA

The match between the T1 sequence and the C1/C2 sequence is

AAACATATAAAACGGAATGAGGAATAATCGTAATATTAGTATGTAGAAAT
ATAGATTCCATTTTGAGGATTCCTATATCCT

The match between the T2 sequence and the C1/C2 sequence is

AATATTAGTATGTAGAAATATAGATTCCATTTTGAGGATTCCTATATCCTC
GAGGAGAACTTCTAGTATATTCTGTA

A double stranded DNA loop of length 11.020 kilo-bases on chromosome 10 is bounded on the left by a T1 sequence whose identifier is 4295. This T1 control element has the DNA sequence

AAACGCAAGGATTGATAATGTAATAGGATCAATGAATATAAACATATAAA
ACGGAATGAGGAATAATCGTAATATTAGTATGTAGAAATATAGATTCCAT
TTTGAGGATTCCTATATCCTCGAGGAGAACTTCTAGTATATTCTG

This double stranded DNA loop is bounded on the right by a T2 control element whose identifier is 4308. This T2 control element has the DNA sequence

GGAAGCTGAAACGCAAGGATTGATAATGTAATAGGATCAATGAATATAA
ACATATAAAACGGAATGAGGAATAATCGTAATATTAGTATGTAGAAATAT
AGATTCCATTTTGAGGATTCCTATATCCTCGAGGAGAACTTCTAGTATATT
CTGTATACCTAATATTATAGCCTTTATCAA

This long T1/T2 double stranded DNA loop modulates the expression of the following genes

YJR027W YJR029W

The expression of genes in this T1/T2 long loop is controlled by the following C1/C2 short loops.

A C1/C2 short loop on chromosome 1 whose identifier is 87 controls the expression of the genes in this T1/T2 long loop. This C1/C2 short loop is expressed as a RNA single strand that is 3′UTR to the gene YAR009C and has the DNA sequence

ATCTATTACATTATGGGTGGTATGTTGGAATAGAAATCAACTATCATCTAC
TAACTAGTATTTACATTACTAGTATATTATCATATACGGTGTTAGAAGATG
ACGCAAATGATGAGAAATAGTCATCTAAATTAGTGGAAGCTGAAACGCA
AGGATTGATAATGTAATAGGATCAATGAATATAAACATATAAAACGGAAT
GAGGAATAATCGTAATATTAGTATGTAGAAATATAGATTCCATTTTGAGC
ATTCCTATATCCTCGAGGAGAACTTCTAGTATATTCTGTATACCTAATATT
ATAGCCTTTATCAACAATGGAATCCCAACAATTATCTCAACATTCACCCAT
TTCTCA

A double stranded DNA loop of length 5.462 kilo-bases on chromosome 13 is bounded on the left by a T1 sequence whose identifier is 5916. This T1 control element has the DNA sequence

AAGCTGAAGTGCAAGGATTGATAATGTAATAGGATAATGAAACATATAA
AACGGAATGAGGAATAATCGTAATATTAGTATGTAGAAATATAGATTCCA
TTTTGAGGATTCCTATATCCTCGAGGAGAACTTCTAGTATATTCTGTA

This double stranded DNA loop is bounded on the right by a T2 control element whose identifier is 5923. This T2 control element has the DNA sequence

TAATAGGATAATGAAACATATAAAACGGAATGAGGAATAATCGTAATATT
AGTATGTAGAAATATAGATTCCATTTTGAGGATTCCTATATCCTCGAGGAG
AACTTCTAGTATATTCTGTATACCTAATATTATAGCCTTTATCAA

This long T1/T2 double stranded DNA loop modulates the expression of the following genes

YML045W

The expression of genes in this T1/T2 long loop is controlled by the following C1/C2 short loops.

A C1/C2 short loop on chromosome 1 whose identifier is 87 controls the expression of the genes in this T1/T2 long loop. This C1/C2 short loop is expressed as a RNA single strand that is 3′UTR to the gene YAR009C and has the DNA sequence

ATCTATTACATTATGGGTGGTATGTTGGAATAGAAATCAACTATCATCTAC
TAACTAGTATTTACATTACTAGTATATTATCATATACGGTGTTAGAAGATG
ACGCAAATGATGAGAAATAGTCATCTAAATTAGTGGAAGCTGAAACGCA
AGGATTGATAATGTAATAGGATCAATGAATATAAACATATAAAACGGAAT
GAGGAATAATCGTAATATTAGTATGTAGAAATATAGATTCCATTTTGAGG
ATTCCTATATCCTCGAGGAGAACTTCTAGTATATTCTGTATACCTAATATT
ATAGCCTTTATCAACAATGGAATCCCAACAATTATCTCAACATTCACCCAT
TTCTCA

Example of a One-to-many Connectron in Multi-cell Eukaryotes—C. elegans

In this example the existence of T1-T2 (16554-16661 and 21565-21590) long loops are controlled by one C1/C2 short loop (21591).

```
                            21591 Chromosome 5
                            |
*------------------*------------------*
| Chromosome 4  |
16554  16661
                            21591 Chromosome 5
                            |
*------------------*------------------*
| Chromosome 5  |
21565  21590
---------------------------------------------------------
```

A double stranded DNA loop of length 50.159 kilo-bases on chromosome 4 is bounded on the left by a T1 sequence whose identifier is 16554. This T1 control element has the DNA sequence

TGCCTGAAAAAATTGGCTCCGAGTTAGGACACTTGGGGTGGTCAAAAAT
TTTGTGACTATTGTCAAATGAAAGATCATAGTTGATAACATAAATTCCCAA
AGTTTCATAAAAATCGATACGCAGCGAACAAAGTTATCAATT

This double stranded DNA loop is bounded on the right by a T2 control element whose identifier is 16661. This T2 control element has the DNA sequence

CACTTGGGGTGGTCAAAAAATTTTGTGATTATTGTCAAATGAAAGATCAT
GGTTGATAACATAAATTCCCAAAGTTTCATAAAAATCGATACGCAGCGAA
CAAAGTTATGATTTTTGACCCGGAACTTATTTGGAGACCTA

This long T1/T2 double stranded DNA loop modulates the expression of the following genes

C23H5.7 C23H5.8a C23H5.3 C23H5.2 C23H5.9 C23H5.1

The expression of genes in this T1/T2 long loop is controlled by the following C1/C2 short loops.

A C1/C2 short loop on chromosome 5 whose identifier is 21591 controls the expression of the genes in this T1/T2 long loop. This C1/C2 short loop is expressed as a RNA single strand that is 3′UTR to the gene F25A2.1 and has the DNA sequence

TATTGTCAAATGAAAGATCATGGTTGATAACATAAATTCCCACAATTTCAT
AAAAATCGATACGCAGCGAACAAAGTTATGATTTTTGACCCGGAACTTAT
TTGGAGACCTAATATT

The match between the T1 sequence and the C1/C2 sequence is

TTTCATAAAAATCGATACGCAGCGAACAAAGTTAT

The match between the T2 sequence and the C1/C2 sequence is

TATTGTCAAATGAAAGATCATGGTTGATAACATAAATTCCCA

A double stranded DNA loop of length 18.142 kilo-bases on chromosome 5 is bounded on the left by a T1 sequence whose identifier is 21565. This T1 control element has the DNA sequence

CTCCGAGTTAGGACACTTGGGGTGGACAAAAAATTTTGTGACTATTGTCA
AATGAAAGATCATGGTTGATAA

This double stranded DNA loop is bounded on the right by a T2 control element whose identifier is 21590. This T2 control element has the DNA sequence

TATTGTCAAATGAAAGATCATGGTTGATAACATAAATTCCCACAATTTCAT
AAAAATCGATACGCAGCGAACAAAGTTATGATTTTTGACCCGGAACTTAT
TTGGAGACCTAATA

This long T1/T2 double stranded DNA loop modulates the expression of the following genes

T21H3.2 T21H3.1 F25A2.1

The expression of genes in this T1/T2 long loop is controlled by the following C1/C2 short loops.

A C1/C2 short loop on chromosome 5 whose identifier is 21591 controls the expression of the genes in this T1/T2 long loop. This C1/C2 short loop is expressed as a RNA single strand that is 3′UTR to the gene F25A2.1 and has the DNA sequence

TATTGTCAAATGAAAGATCATGGTTGATAACATAAATTCCCACAATTTCAT
AAAAATCGATACGCAGCGAACAAAGTTATGATTTTTGACCCGGAACTTAT
TTGGAGACCTAATATT

The match between the T1 sequence and the C1/C2 sequence is

TATTGTCAAATGAAAGATCATGGTTGATAA

The match between the T2 sequence and the C1/C2 sequence is

TATTGTCAAATGAAAGATCATGGTTGATAACATAAATTCCCACAATTTCAT
AAAAATCGATACGCAGCGAACAAAGTTATGATTTTTGACCCGGAACTTAT
TTGGAGACCTAATA

4. Connectrons Occur Between Prokaryotes and Their Plasmids.

Connectron relationships exist between prokaryotes and their plasmids. These connectrons implement a control mechanism between the two genomes that makes it possible for them to form a symbiotic relationship. In the case of D. radiodurans the relationship is not symmetric. The D. radiodurans genome sends C1/C2 short loops to the MP1 plasmid.

Example of a Prokaryote/Plasmid Connectron—D. radiodurans

In this example the existence of T1-T2 (2654-2694 and 2692-2749) long loops in chromosome 3 that is the plasmid MP1 are controlled by one C1/C2 short loop (16) in chromosome 1.

```
                            16 Chromosome 1
                            2768 Chromosome 3 (plasmid MP1)
                            2653 Chromosome 3 (plasmid MP1)
                            |
*------------------*------------------*
| Chromosome 3 (plasmid MP1)  |
2654  2694
| 2693  |
                            16 Chromosome 1
                            2768 Chromosome 3 (plasmid MP1)
                            2693 Chromosome 3 (plasmid MP1)
                            |
*------------------*------------------*
| Chromosome 3 (plasmid MP1)  |
2692  2749
| 2693  2695  |
--------------------------------------------------------
```

A double stranded DNA loop of length 46.903 kilo-bases on chromosome 3 (plasmid MP1) is bounded on the left by a T1 sequence whose identifier is 2654. This T1 control element has the DNA sequence

CAGCGTTTTTCTCGCTGTTCCTGGACGGCTGAACGCCCTGAATCTCTCCCG
GTATAGCAGCCTGCTCGGAGAGTACGATTCGTCGTTGGCTGCACCGAAGTG
ACGATGGGGCCATTCCGTGGGGCGCGTTACACCAGGCGACTGTCAGTACA
GCAATCGAGAGTGGGCTGATCAGCCCACTGTGCGTTCTGGCCATCGACGC
CTCTTTTCACCGCAAAGCCGGTCAGCACACCGCACACCTCGGCTCGTTCTG
GAATGGCTGTGCCGCGCGGACC

This double stranded DNA loop is bounded on the right by a T2 control element whose identifier is 2694. This T2 control element has the DNA sequence

GCTGAACGCCCTGAATCTCTCCCGGTATGCAGCCTGCTCGGAGAGTACGA
TTCGTCGTTGGCTGCACCGAAGTGACGATGGGGCCATTCCGTGGGGCGCG
TTACACCAGGCGACTGTCAGTACAGCAATCGAGAGTGGGCTGATCAGCCC
ACTGTGCGTTCTGGCCATCGACGCCTCTTTTCACCGCAAAGCCGGTCAGCA
CACCGCACACCTCGGCTCGTTCTGGAATGGCTGTGCCGCGCGGACCGAAC
GCGGAATCGAGCAATCCTGTTGT

This long T1/T2 double stranded DNA loop modulates the expression of the following genes

| | | | | | |
|---|---|---|---|---|---|
| DRB0020 | DRB0021 | DRB0022 | DRB0023 | DR80024 | DRB0025 |
| DRB0027 | DRB0030 | DRB0032 | DRB0033 | DRB0034 | DRB0035 |
| DRB0037 | DRB0038 | DRB0039 | DRB0041 | DRB0042 | DRB0043 |
| DRB0044 | DRB0045 | DRB0047 | DRB0051 | DRB0052 | DRB0054 |
| DRB0055 | DRB0057 | | | | |

This long T1/T2 double stranded DNA loop modulates the expression of the following C1/C2 short loops

A C1/C2 short loop on chromosome 3 (plasmid MP1) whose identifier is 2693 controls the expression of the genes of one or more other T1/T2 long loops. This C1/C2 short loop is expressed as a RNA single strand that is 3′UTR to the gene DRB0057 and has the DNA sequence

CTGATGGCCATCCTACAGTACGTTCTCAGCGCGGTCCCGCTGCGCAAGAC
GCAGCGGAATTTCCTGACCGTGCTGCTCAGCGTTTTTCTCGCTGTTCCTGG
AC

The expression of genes in this T1/T2 long loop is controlled by the following C1/C2 short loops.

A C1/C2 short loop on chromosome 1 whose identifier is 16 controls the expression of the genes in this T1/T2 long loop. This C1/C2 short loop is expressed as a RNA single strand that is 3′UTR to the gene DR0009 and has the DNA sequence

GCTGTGAAATCACCGCTTCCAATGGGTCTGATGGCCATCCTACAGTACGTT
CTCAGCGCGGTCCCGCTGCGCAAGACGCAGCGGAATTTCCTGACCGTGCT
GCTCAGCGTTTTTCTCGCTGTTCCTGGACGGCTGAACGCCCTGAATCTCTC
CCGGTATGCAGCCTGCTCGGAGAGTACGATTCGT

The match between the T1 sequence and the C1/C2 sequence is

CAGCGTTTTTCTCGCTGTTCCTGGACGGCTGAACGCCCTGAATCTCTCCCG
GTATGCAGCCTGCTCGGAGAGTACGATTCGTCGTTGGCTGCACCGAAGTG
ACGATGGGGCCATTCCGTGGGGCGCGTTACACCAGGCGACTGTCAGTACA
GCAATCGAGAGTGGGCTGATCAGCCCACTGTGCGTTCTGGCCATCGACGC
CTCTTTTCACCGCAAAGCCGGTCAGCACACCGCACACCTCGGCTCGTTCTG
GAATGGCTGTGCCGCGCGGACC

The match between the T2 sequence and the C1/C2 sequence is

GCTGAACGCCCTGAATCTCTCCCGGTATGCAGCCTGCTCGGAGAGTACGA
TTCGTCGTTGGCTGCACCGAAGTGACGATGGGGCCATTCCGTGGGGCGCG
TTACACCAGGCGACTGTCAGTACAGCAATCGAGAGTGGGCTGATCAGCCC
ACTGTGCGTTCTGGCCATCGACGCCTCTTTTCACCGCAAAGCCGGTCAGCA
CACCGCACACCTCGGCTCGTTCTGGAATGGCTGTGCCGCGCGGACCGAAC
GCGGAATCGAGCAATCCTGTTGT

A C1/C2 short loop on chromosome 3 (plasmid MP1) whose identifier is 2768 controls the expression of the genes in this T1/T2 long loop. This C1/C2 short loop is expressed as a RNA single strand that is 3′UTR to the gene DRB0133 and has the DNA sequence

GCTGTGAAATCACCGCTTCCAATGGGTCTGATGGCCATCCTACAGTACGTT
CTCAGCGCGGTCCCGCTGCGCAAGACGCAGCGGAATTTCCTGACCGTGCT
GCTCAGCGTTTTTCTCGCTGTTCCTGGACGGCTGAACGCCCTGAATCTCTC
CCGGTATGCAGCCTGCTCGGAGAGTACGATTCGT

The match between the T1 sequence and the C1/C2 sequence is

CAGCGTTTTTCTCGCTGTTCCTGGACGGCTGAACGCCCTGAATCTCTCCCG
GTATGCAGCCTGCTCGGAGAGTACGATTCGTCGTTGGCTGCACCGAAGTG
ACGATGGGGCCATTCCGTGGGGCGCGTTACACCAGGCGACTGTCAGTACA
GCAATCGAGAGTGGGCTGATCAGCCCACTGTGCGTTCTGGCCATCGACGC
CTCTTTTCACCGCAAAGCCGGTCAGCACACCGCACACCTCGGCTCGTTCTG
GAATGGCTGTGCCGCGCGGACC

The match between the T2 sequence and the C1/C2 sequence is

GCTGAACGCCCTGAATCTCTCCCGGTATGCAGCCTGCTCGGAGAGTACGA
TTCGTCGTTGGCTGCACCGAAGTGACGATGGGGCCATTCCGTGGGGCGCG
TTACACCAGGCGACTGTCAGTACAGCAATCGAGAGTGGGCTGATCAGCCC
ACTGTGCGTTCTGGCCATCGACGCCTCTTTTCACCGCAAAGCCGGTCAGCA
CACCGCACACCTCGGCTCGTTCTGGAATGGCTGTGCCGCGCGGACCGAAC
GCGGAATCGAGCAATCCTGTTGT

A C1/C2 short loop on chromosome 3 (plasmid MP1) whose identifier is 2653 controls the expression of the genes in this T1/T2 long loop. This C1/C2 short loop is expressed as a RNA single strand that is 3′UTR to the gene DRB0017 and has the DNA sequence

CGGTCCCGCTGCGCAAGACGCAGCGGAATTTCCTGACCGTGCTGCTCAGC
GTTTTTCTCGCTGTTCCTGGACGGCTGAACGCCCTGAATCTCTCCCGGTAT
GCAGCCTGCTCGGAGAGTACGATTCGTCGTTGGCTGCACCGAAGTGACGA
TGGGGCCATTCCGTGGGGCGCGTTACACCAGGCGA

The match between the T1 sequence and the C1/C2 sequence is

CAGCGTTTTTCTCGCTGTTCCTGGACGGCTGAACGCCCTGAATCTCTCCCG
GTATGCAGCCTGCTCGGAGAGTACGATTCGTCGTTGGCTGCACCGAAGTG
ACGATGGGGCCATTCCGTGGGGCGCGTTACACCAGGCGACTGTCAGTACA
GCAATCGAGAGTGGGCTGATCAGCCCACTGTGCGTTCTGGCCATCGACGC
CTCTTTTCACCGCAAAGCCGGTCAGCACACCGCACACCTCGGCTCGTTCTG
GAATGGCTGTGCCGCGCGGACC

The match between the T2 sequence and the C1/C2 sequence is

GCTGAACGCCCTGAATCTCTCCCGGTATGCAGCCTGCTCGGAGAGTACGA
TTCGTCGTTGGCTGCACCGAAGTGACGATGGGGCCATTCCGTGGGGCGCG
TTACACCAGGCGACTGTCAGTACAGCAATCGAGAGTGGGCTGATCAGCCC
ACTGTGCGTTCTGGCCATCGACGCCTCTTTTCACCGCAAAGCCGGTCAGCA
CACCGCACACCTCGGCTCGTTCTGGAATGGCTGTGCCGCGCGGACCGAAC
GCGGAATCGAGCAATCCTGTTGT

A double stranded DNA loop of length 68.612 kilo-bases on chromosome 3 (plasmid MP1) is bounded on the left by a T1 sequence whose identifier is 2692. This T1 control element has the DNA sequence

CTGATGGCCATCCTACAGTACGTTCTCAGCGCGGTCCCGCTGCGCAAGAC
GCAGCGGAATTTCCTGACCGTGCTGCTCAGCGTTTTTCTCGCTGTTCCTGG
AC

This double stranded DNA loop is bounded on the right by a T2 control element whose identifier is 2749. This T2 control element has the DNA sequence

AGCGCGGTCCCGCTGCGCAAGACGCAGCGGAATTTCCTGACCGTGCTGCT
CAGCGTTTTTCTCGCTGTTCCTGGACGGCTGAACGCCCTGAATCTCTCCCG
GT

This long T1/ T2 double stranded DNA loop modulates the expression of the following genes

| | | | | | |
|---|---|---|---|---|---|
| DRB0059 | DRB0060 | DRB0061 | DRB0062 | DRB0064 | DRB0065 |
| DRB0066 | DRB0067 | DRB0068 | DRB0069 | DRB0070 | DRB0072 |
| DRB0073 | DRB0074 | DRB0076 | DRB0077 | DRB0079 | DRB0080 |
| DRB0081 | DRB0083 | DRB0085 | DRB0086 | DRB0087 | DRB0088 |
| DRB0089 | DRB0090 | DRB0092 | DRB0093 | DRB0094 | DRB0096 |
| DRB0097 | DRB0098 | DRB0102 | DRB0103 | DRB0104 | DRB0105 |
| DRB0106 | DRB0107 | DRB0111 | DRB0112 | | |

This long T1/T2 double stranded DNA loop modulates the expression of the following C1/C2 short loops

A C1/C2 short loop on chromosome 3 (plasmid MP1) whose identifier is 2693 controls the expression of the genes of one or more other T1/T2 long loops. This C1/C2 short loop is expressed as a RNA single strand that is 3′UTR to the gene DRB0057 and has the DNA sequence

CTGATGGCCATCCTACAGTACGTTCTCAGCGCGGTCCCGCTGCGCAAGAC
GCAGCGGAATTTCCTGACCGTGCTGCTCAGCGTTTTTCTCGCTGTTCCTGG
AC

A C1/C2 short loop on chromosome 3 (plasmid MP1) whose identifier is 2695 controls the expression of the genes of one or more other T1/T2 long loops. This C1/C2 short loop is expressed as a RNA single strand that is 3′UTR to the gene DRB0057 and has the DNA sequence

GCTGAACGCCCTGAATCTCTCCCGGTATGCAGCCTGCTCGGAGAGTACGA
TTCGTCGTTGGCTGCACCGAAGTGACGATGGGGCCATTCCGTGGGGCGCG
TTACACCAGGCGACTGTCAGTACAGCAATCGAGAGTGGGCTGATCAGCCC
ACTGTGCGTTCTGGCCATCGACGCCTCTTTTCACCGCAAAGCCGGTCAGCA
CACCGCACACCTCGGCTCGTTCTGGAATGGCTGTGCCGCGCGGACCGAAC
GCGGAATCGAGCAATCCTGTTGT

The expression of genes in this T1/T2 long loop is controlled by the following C1/C2 short loops.

A C1/C2 short loop on chromosome 1 whose identifier is 16 controls the expression of the genes in this T1/T2 long loop. This C1/C2 short loop is expressed as a RNA single strand that is 3′UTR to the gene DR0009 and has the DNA sequence

GCTGTGAAATCACCGCTTCCAATGGGTCTGATGGCCATCCTACAGTACGTT
CTCAGCGCGGTCCCGCTGCGCAAGACGCAGCGGAATTTCCTGACCGTGCT
GCTCAGCGTTTTTCTCGCTGTTCCTGGACGGCTGAACGCCCTGAATCTCTC
CCGGTATGCAGCCTGCTCGGAGAGTACGATTCGT

The match between the T1 sequence and the C1/C2 sequence is

CTGATGGCCATCCTACAGTACGTTCTCAGCGCGGTCCCGCTGCGCAAGAC
GCAGCGGAATTTCCTGACCGTGCTGCTCAGCGTTTTTCTCGCTGTTCCTGG
AC

The match between the T2 sequence and the C1/C2 sequence is

AGCGCGGTCCCGCTGCGCAAGACGCAGCGGAATTTCCTGACCGTGCTGCT
CAGCGTTTTTCTCGCTGTTCCTGGACGGCTGAACGCCCTGAATCTCTCCCG
GT

A C1/C2 short loop on chromosome 3 (plasmid MP1) whose identifier is 2768 controls the expression of the genes in this T1/T2 long loop. This C1/C2 short loop is expressed as a RNA single strand that is 3′UTR to the gene DRB0133 and has the DNA sequence

GCTGTGAAATCACCGCTTCCAATGGGTCTGATGGCCATCCTACAGTACGTT
CTCAGCGCGGTCCCGCTGCGCAAGACGCAGCGGAATTTCCTGACCGTGCT
GCTCAGCGTTTTTCTCGCTGTTCCTGGACGGCTGAACGCCCTGAATCTCTC
CCGGTATGCAGCCTGCTCGGAGAGTACGATTCGT...CGGACCGAACGCGGA
ATCGAGCAATCCTGTTGTGCCCTCATTGATGTCCAGCACCGGCAGGCCTTG
ACGGTCGATGTCCGTCAGACCCTGACCGGGTCTGAGGCTCCAACTCGTCT
GGAACAG

The match between the T1 sequence and the C1/C2 sequence is

CTGATGGCCATCCTACAGTACGTTCTCAGCGCGGTCCCGCTGCGCAAGAC
GCAGCGGAATTTCCTGACCGTGCTGCTCAGCGTTTTTCTCGCTGTTCCTGG
AC

The match between the T2 sequence and the C1/C2 sequence is

AGCGCGGTCCCGCTGCGCAAGACGCAGCGGAATTTCCTGACCGTGCTGCT
CAGCGTTTTTCTCGCTGTTCCTGGACGGCTGAACGCCCTGAATCTCTCCCG
GT

A C1/C2 short loop on chromosome 3 (plasmid MP1) whose identifier is 2693 controls the expression of the genes in this T1/T2 long loop. This C1/C2 short loop is expressed as a RNA single strand that is 3′UTR to the gene DRB0057 and has the DNA sequence

CTGATGGCCATCCTACAGTACGTTCTCAGCGCGGTCCCGCTGCGCAAGAC
GCAGCGGAATTTCCTGACCGTGCTGCTCAGCGTTTTTCTCGCTGTTCCTGG
AC

The match between the T1 sequence and the C1/C2 sequence is

CTGATGGCCATCCTACAGTACGTTCTCAGCGCGGTCCCGCTGCGCAAGAC
GCAGCGGAATTTCCTGACCGTGCTGCTCAGCGTTTTTCTCGCTGTTCCTGG
AC

The match between the T2 sequence and the C1/C2 sequence is

AGCGCGGTCCCGCTGCGCAAGACGCAGCGGAATTTCCTGACCGTGCTGCT
CAGCGTTTTTCTCGCTGTTCCTGGAC

A C1/C2 short loop on chromosome 3 (plasmid MP1) whose identifier is 2653 controls the expression of the genes in this T1T2 long loop. This C1/C2 short loop is expressed as a RNA single strand that is 3′UTR to the gene DRB0017 and has the DNA sequence

CGGTCCCGCTGCGCAAGACGCAGCGGAATTTCCTGACCGTGCTGCTCAGC
GTTTTTCTCGCTGTTCCTGGACGGCTGAACGCCCTGAATCTCTCCCGGTAT
GCAGCCTGCTCGGAGAGTACGATTCGTCGTTGGCTGCACCGAAGTGACGA
TGGGGCCATTCCGTGGGGCGCGTTACACCAGGCGA

The match between the T1 sequence and the C1/C2 sequence is

CGGTCCCGCTGCGCAAGACGCAGCGGAATTTCCTGACCGTGCTGCTCAGC
GTTTTTCTCGCTGTTCCTGGAC

The match between the T2 sequence and the C1/C2 sequence is

CGGTCCCGCTGCGCAAGACGCAGCGGAATTTCCTGACCGTGCTGCTCAGC
GTTTTTCTCGCTGTTCCTGGACGGCTGAACGCCCTGAATCTCTCCCGGT

5. Connectrons Occur in Plants and Higher Animals

Connectron relationships exist in plant and higher animals.

Example of a Plant Connectron—A. thaliania

In this example the existence of the T1-T2 (423-469) long loop is controlled by six C1/C2 short loops (972, 21396, 422, 21762, 21813 and 10882). The T1-T2 long loop controls the expression of six genes on chromosome 2 in addition to two C1/C2 (426 and 430) short loops.

```
                          972 Chromosome 2
                          21396 Chromosome 4
                          422 Chromosome 2
                          21762 Chromosome 4
                          21813 Chromosome 4
                          10882 Chromosome 4
                          |
*------------------*------------------*
| Chromosome 2  |
423  469
| 426  430  |
-------------------------------------------------------
```

A double stranded DNA loop of length 42.285 kilo-bases on chromosome 2 is bounded on the left by a T1 sequence whose identifier is 423. This T1 control element has the DNA sequence

TATCTCTTTAAGGATTAAAAAGTCAAATACTAATTTAATTAATTAAATTTA
ATTAAAAAACGAAATA

This double stranded DNA loop is bounded on the right by a T2 control element whose identifier is 469. This T2 control element has the DNA sequence

TACTAATTTAATTAATTAAATTTAATTAAAAAACGAAATACATTATTAATT
TTCAAAAATAATAACC

This long T1/T2 double stranded DNA loop modulates the expression of the following genes

At2g02070 At2g02080 At2g02090 At2g02100 At2g02120 At2g02130

This long T1/T2 double stranded DNA loop modulates the expression of the following C1/C2 short loops

A C1/C2 short loop on chromosome 2 whose identifier is 426 controls the expression of the genes of one or more other T1/T2 long loops. This C1/C2 short loop is expressed as a RNA single strand that is 3′UTR to the gene At2g02060 and has the DNA sequence

TTCCAAAAATAATAACCAATCAAAATCAACATATAAGATTTGATATCTAA
ATTTT

A C1/C2 short loop on chromosome 2 whose identifier is 430 controls the expression of the genes of one or more other T1/T2 long loops. This C1/C2 short loop is expressed as a RNA single strand that is 3′UTR to the gene At2g02060 and has the DNA sequence

TTGCGGAAAAATAATATCATCATTATAAAAAAATAATTAGAGTTTTTTCG
CATAT

The expression of genes in this T1/T2 long loop is controlled by the following C1/C2 short loops.

A C1/C2 short loop on chromosome 2 whose identifier is 972 controls the expression of the genes in this T1/T2 long loop. This C1/C2 short loop is expressed as a RNA single strand that is 3′UTR to the gene At2g04240 and has the DNA sequence

GTATGCCATTAGAAATAAAATTTTAAAAGTAAATTAATTCATCTCTTTAAA
AATTAAAAAGTCAAATACTAATTTAATTAATTAAATTTAATTAAAAAACG
AAATACATTATTAATTT

The match between the T1 sequence and the C1/C2 sequence is

ATTAAAAAGTCAAATACTAATTTAATTAATTAAATTTAATTAAAAAACGA
AATA

The match between the T2 sequence and the C1/C2 sequence is

TACTAATTTAATTAATTAAATTTAATTAAAAAACGAAATACATTATTAAT
TT

A C1/C2 short loop on chromosome 4 whose identifier is 21396 controls the expression of the genes in this T1/T2 long loop. This C1/C2 short loop is expressed as a RNA single strand that is 3′UTR to the gene AT4g15300 and has the DNA sequence

TGCCATTAGAAATAAAATTTTAAAGAGTAAATTAATTTATCTCTTTAAGGA
TTAAAAAGTCAAATACTAATTTAATTAATTAAATTTAATTAAAAAACGAA
ATACATTATTAATTTCCAAAA

The match between the T1 sequence and the C1/C2 sequence is

TATCTCTTTAAGGATTAAAAAGTCAAATACTATTTAATTAATTAAATTTA
ATTAAAAAACGAAATA

The match between the T2 sequence and the C1/C2 sequence is

TACTAATTTAATTAATTAAATTTAATTAAAAAACGAAATACATTATTAATT
T

A C1/C2 short loop on chromosome 2 whose identifier is 422 controls the expression of the genes in this T1/T2 long loop. This C1/C2 short loop is expressed as a RNA single strand that is 3′UTR to the gene At2g02060 and has the DNA sequence

TAACCTTAATTTTTGTAAGTAATTATATAGGTATGCCATTAGAAATAAAAT
TTTAAAGAGTAAATTAATTTATCTCTTTAAGGATTAAAAAGTCAAATACTA
ATTTAATTAATTAAATTTAATTAAAAAACGAAATA

The match between the T1 sequence and the C1/C2 sequence is

TATCTCTTTAAGGATTAAAAAGTCAAATACTAATTTAATTAATTAAATTTA
ATTAAAAAACGAAATA

The match between the T2 sequence and the C1/C2 sequence is

TACTAATTTAATTAATTAAATTTAATTAAAAAACGAAATA

A C1/C2 short loop on chromosome 4 whose identifier is 21762 controls the expression of the genes in this T1/T2 long loop. This C1/C2 short loop is expressed as a RNA single strand that is 3′UTR to the gene AT4g17510 and has the DNA sequence

TTTAAGGATTAAAAAGTCAAATACTAATTTAATTAATTAAATTTAATTAAA
AAACGAAATACATT

The match between the T1 sequence and the C1/C2 sequence is

TTTAAGGATTAAAAAGTCAAATACTAATTTAATTAATTAAATTTAATTAAA
AAACGAAATA

The match between the T2 sequence and the C1/C2 sequence is

TACTAATTTAATTAATTAAATTTAATTAAAAAACGAAATACATT

A C1/C2 short loop on chromosome 4 whose identifier is 21813 controls the expression of the genes in this T1/T2 long loop. This C1/C2 short loop is expressed as a RNA single strand that is 3′UTR to the gene AT4g17680 and has the DNA sequence

TTTAAGGATTAAAAAGTCAAATACTAATTTAATTAATTAAATTTAATTAAA
AAACGAAATACATT

The match between the T1 sequence and the C1/C2 sequence is

TTTAAGGATTAAAAAGTCAAATACTAATTTAATTAATTAAATTTAATTAAA
AAACGAAATA

The match between the T2 sequence and the C1/C2 sequence is

TACTAATTTAATTAATTAAATTTAATTAAAAAACGAAATACATT

A C1/C2 short loop on chromosome 2 whose identifier is 10882 controls the expression of the genes in this T1/T2 long loop. This C1/C2 short loop is expressed as a RNA single strand that is 3′UTR to the gene At2g26540 and has the DNA sequence

TATCTCTTTAAGGATTAAAAAGTCAAATACTAATTTAATTAATTAAATTTA
ATTAA

The match between the T2 sequence and the C1/C2 sequence is

TACTAATTTAATTAATTAAATTTAATTAA

Example of a Animal Connectron—D. megalomaster

A double stranded DNA loop of length 88.159 kilo-bases on chromosome 4 is bounded on the left by a T1 sequence whose identifier is 3340. This T1 control element has the DNA sequence

ACCTAAAAGAAGTACCGTTTTTTACTCCTAATTACCAATTCTAACCATCCA
TATCACTTTTTGACGGACTCCGTGAAAATAATTTTTGGCCAAATTTTCGCA
TTTTTTGTAAGGGGTAACATCATAAAAATT

This double stranded DNA loop is bounded on the right by a T2 control element whose identifier is 3372. This T2 control element has the DNA sequence

AAAAAAGTACCGCGTTTTACTCCTAATTACCAATTCTAACCATCCATATCA
CTTTTTGACGGACTCCGTGAAAATAATTTTTGGCCAAATTTTCGCATTTTTT
GTAAGGGGTAACATCATCAAAATTTGCGAAAAA

This long T1/T2 double stranded DNA loop modulates the expression of the following genes

[Some of the Following Gene Names have not been Determined.]

| | | | | |
|---|---|---|---|---|
| — | — | — | — | — |
| — | CG11207 | — | CG2186 | CG2157 |
| — | Ork1 | — | — | — |
| — | | | | |

This long T1/T2 double stranded DNA loop modulates the expression of the following C1/C2 short loops

A C1/C2 short loop on chromosome 4 whose identifier is 3362 controls the expression of the genes of one or more other T1/T2 long loops. This C1/C2 short loop is expressed as a RNA single strand that is 3′UTR to the gene XXX and has the DNA sequence

AAAAAAGTACCGCGTTTTACTCCTAATTACCAATTCTAACCATCCATATCA
CTTTTTGACGGACTCCGTTAAAATAATTTTTGACCAAATTTTCGCATTTTTT
GTAATCAAAATTTGCAAAAAATTGAAAAAAC

A C1/C2 short loop on chromosome 4 whose identifier is 3364 controls the expression of the genes of one or more other T1/T2 long loops. This C1/C2 short loop is expressed as a RNA single strand that is 3′UTR to the gene XXX and has the DNA sequence

CAAAATTTGAATGCAAATCGATTGGGAATCAAAAAACAAACTCAACGAG
GTATGACATTCCATATTTGGGCCATTATTTCCAA

A C1/C2 short loop on chromosome 4 whose identifier is 3366 controls the expression of the genes of one or more other T1/T2 long loops. This C1/C2 short loop is expressed as a RNA single strand that is 3′UTR to the gene XXX and has the DNA sequence

TTTTTTCACAAAAATTAGGAAAATGATTTTGGGTAAAAAAATGAATATTT
AAGTTGGGTTTT

A C1/C2 short loop on chromosome 4 whose identifier is 3369 controls the expression of the genes of one or more other T1/T2 long loops. This C1/C2 short loop is expressed as a RNA single strand that is 3′UTR to the gene XXX and has the DNA sequence

AAATCGATTGGGAATCAAAAAACAAACCTCAACGAGGTATGACATTCCAT
ATCTGGGCCATTATTTCCAATCTTTTGATCAAAATAC

The expression of genes in this T1/T2 long loop is controlled by the following C1/C2 short loops.

A C1/C2 short loop on chromosome 4 whose identifier is 3373 controls the expression of the genes in this T1/T2 long loop. This C1/C2 short loop is expressed as a RNA single strand that is 3′UTR to the gene XXX and has the DNA sequence

AAAAAAGTACCGCGTTTTACTCCTAATTACCAATTCTAACCATCCATATCA
CTTTTTGACGGACTCCGTGAAAATAATTTTTGGCCAAATTTTCGCATTTTTT
GTAAGGGGTAACATCATCAAAATTTGCGAAAAA

The match between the T1 sequence and the C1/C2 sequence is

TTTTACTCCTAATTACCAATTCTAACCATCCATATCACTTTTTGACGGACTC
CGTGAAAATAATTTTTGGCCAAATTTTCGCATTTTTTGTAAGGGGTAACAT
CAT

Example of an Animal Connectron—H. sapiens

All of the human genome that has been fully sequenced by both the NIH-lead global sequencing project and the Celera Genomics, Inc. project. The gene descriptors for this chromosome do not yet exist. Without the positions and directions of the genes, it is not possible to select from among the possible connectrons to determine the real connectrons.

Human chromosome 22 has been processed and there 31,000 possible connectrons.

The gene descriptors for all the chromosomes of the human genome should become available within the year.

6. Permanent Connectrons Exist in Prokaryotes, Archea, Single-celled Eukaryotes and Multi-celled Eukaryotes.

C1/C2 short loops are normally expressed as the 3′UTR of some gene. A class of connectron relationships exist that permit one C1/C2 short loop to control the existence of one or more T1-T2 long loops without being subject to any expression controls other than those of the gene to which the C1/C2 is 3′UTR. These connectron relationships are described as “permanent”. Permanent connectrons exist in prokaryotes, archea, single-celled eukaryotes and multi-celled eukaryotes.

Example of a Prokaryote Permanent Connectron—E. coli

In this example the existence of the T1-T2 (3200-3210) long loop is controlled by a C1/C2 short loop (3432). The expression of this C1/C2 short loop is controlled only by the gene btuB.

```
                            3432 Chromosome 1
                            |
*------------------*------------------*
| Chromosome 1  |
3200  3210
--------------------------------------------------------
```

A double stranded DNA loop of length 93.339 kilo-bases on chromosome 1 is bounded on the left by a T1 sequence whose identifier is 3200. This T1 control element has the DNA sequence

AAGCGGCACTGCTCTTTAACAATTTATCAGACAATCTGTGTGGGCACTCG
AAGATACGGATTCTTAACGTCGCAAGACGAAAAATGAATACCAAGTCTCA
AGAGTGAACACGTAATTCATTACGAAGTTTAATTCTTTGAGCATCAAACTT
TTAAATTGAAGAGTTTGATCATGGCTCAGATTGAACGCTGGCGGCAGGCC
TAACACATGCAAGTCGAACGGTAACAGGAAACAGCTTGCTGTTTCGCTGA
CGAGTGGCGGACGGGTGAGTAATGTCTGGGAAACTGCCTGATGGAGGGG
GATAACTACTGGAAACGGTAGCTAATACCGCATAACGTCGCAAGACCAAA
GAGGGGGACCTTCGGGCCTCTTGCCATC

This double stranded DNA loop is bounded on the right by a T2 control element whose identifier is 3310. This T2 control element has the DNA sequence

CAGACAATCTGTGTGGGCACTCGAAGATACGGATTCTTAACGTCGCAAGA
CGAAAAATGAATACCAAGTCTCAAGAGTGAACACGTAATTCATTACGAAG
TTTAATTCTTTGAGCGTCAAACTTTTAAATTGAAGAGTTTGATCATGGCTC
AGATTGAACGCTGGCGGCAGGCCTAACACATGCAAGTCGAACGGTAACA
GGAAGAAGCTTGCTTCTTTGCTGACGAGTGGCGGACGGGTGAGTAATGTC
TGGGAAACTGCCTGATGGAGGGGGATAACTACTGGAAACGGTAGCTAAT
ACCGCATAACGTCGCAAGACCAAAGAGGGGGACCTTCGGGCCTCTTGCCA
TCGGATGTGCCCAGATGGGATTAGCTAGT

This long T1/T2 double stranded DNA loop modulates the expression of the following genes

| | | | | | | | | |
|---|---|---|---|---|---|---|---|---|
| rrsC | gltU | rrlC | rrfC | aspT | trpT | yifA | yifE | yifB |
| ilvL | ilvG_1 | ilvM | ilvE | ilvD | ilvA | ilvY | ilvC | ppiC |
| b3776 | rep | gppA | rhlB | trxA | rhoL | rho | rfe | wzzE |
| wecB | rffH | wecD | wecE | wzxE | yifM_2 | wecG | yifK | |
| argX | hisR | leuT | proM | aslB | aslA | hemY | hemX | |
| hemD | cyaA | cyaY | b3808 | dapF | uvrD | b3814 | corA | |
| yigF | yigG | rarD | yigI | pldA | recQ | yigJ | yigK | pldB |
| yigL | yigM | metR | metE | ysgA | udp | yigN | ubiE | yigP |
| b3836 | yigU | yigW_1 | rfaH | yigC | ubiB | fadA | fadB | |
| pepQ | trkH | hemG | | | | | | |

The expression of genes in this T1/T2 long loop is controlled by the following C1/C2 short loops.

A C1/C2 short loop on chromosome 1 whose identifier is 3432 controls the expression of the genes in this T1/T2 long loop. This C1/C2 short loop is expressed as a RNA single strand that is 3′UTR to the gene btuB and has the DNA sequence

TGCGCGGTCAGAAAATTATTTTAAATTTCCTCTTGTCAGGCCGGAATAACT
CCCTATAATGCGCCACCACTGACACGGAACAACGGCAAACACGCCGCCGG
GTCAGCGGGGTTCTCCTGAGAACTCCGGCAGAGAAAGCAAAAATAAATG
CTTGACTCTGTAGCGGGAAGGCGTATTATGCACACC...TGCAACTCGACTC
CATGAAGTCGGAATCGCTAGTAATCGTGGATCAGAATGCCACGGTGAATA
CGTTCCCGGGCCTTGTACACACCGCCCGTCACACCATGGGAGTGGGTTGC
AAAAGAAGTAGGTAGCTTAACCTTCGGGAGGGCGCTTACCACTTTGTGAT
TCATGACTGGGGTGAAGTCGTAACAAGGTAACCGTAGGGGAACCTGCGGT
TGGATCACCTCCTTACCTTAAAGAAGCGT

The match between the T1 sequence and the C1/C2 sequence is

AAGCGGCACTGCTCTTTAACAATTTATCAGACAATCTGTGTGGGCACTCG
AAGATACGGATTCTTAACGTCGCAAGACGAAAAATGAATACCAAGTCTCA
AGAGTGAACACGTAATTCATTACGAAGTTTAATTCTTTGAGC

The match between the T2 sequence and the C1/C2 sequence is

CAGACAATCTGTGTGGGCACTCGAAGATACGGATTCTTAACGTCGCAAGA
CGAAAAATGAATACCAAGTCTCAAGAGTGAACACGTAATTCATTACGAAG
TTTAATTCTTTGAGCGTCAAACTTTTAAATTGAAGAGTTTGATCATGGCTC
AGATTGAACGCTGGCGGCAGGCCTAACACATGCAAGTCGAACGGTAACA
GGAAGAAGCTTGCTTCTTTGCTGACGAGTGGCGGACGGGTGAGTAATGTC
TGGGAAACTGCCTGATGGAGGGGGATAACTACTGGAAACGGTAGCTAAT
ACCGCATAACGTCGCAAGACCAAAGAGGGGGACCTTCGGGCCTCTTGCCA
TCGGATGTGCCCAGATGGGATTAGCTAGT

Example of an Archea Permanent Connectron—H. pylori

In this example the existence of the T1-T2 (812-882) long loop is controlled by a C1/C2 short loop (1241). The expression of this C1/C2 short loop is controlled only by the gene HP1535.

```
                          1241 Chromosome 1
                          |
*------------------*------------------*
| Chromosome 1  |
812  882
--------------------------------------------------------
```

A double stranded DNA loop of length 96.385 kilo-bases on chromosome 1 is bounded on the left by a T1 sequence whose identifier is 812. This T1 control element has the DNA sequence

TTTTACTCATAGGGTTTTTATAGTTCCTAGCGGAACTAAAGCA

This double stranded DNA loop is bounded on the right by a T2 control element whose identifier is 882. This T2 control element has the DNA sequence

TAGCGGAACTAAAGCATTCATCCCAAACACTAAAGATATTTGG

This long T1/T2 double stranded DNA loop modulates the expression of the following genes

| | | | | | | |
|---|---|---|---|---|---|---|
| HP0999 | HP1000 | HP1001 | HP1002 | HP1003 | HP1005 | HP1006 |
| HP1008 | HP1009 | HPtRNA-Pro | HP1010 | HP1011 | HP1013 | HP1015 |
| HP1017 | HP1018 | HP1020 | HP1021 | HP1022 | HP1023 | HP1024 |
| HP1025 | HP1027 | HP1028 | HP1030 | HP1031 | HP1033 | HP1034 |
| HP1038 | HP1039 | HP1040 | HP1041 | HP1042 | HP1043 | HP1044 |
| HP1045 | HP1046 | HP1051 | HP1052 | HP1055 | HP1056 | HP1058 |
| HP1060 | HP1065 | HPtRNA-Ser | HP1066 | HP1067 | HP1069 | HP1070 |
| HP1074 | HP1075 | HP1076 | HP1077 | HP1078 | HP1079 | HP1080 |
| HP1081 | HP1083 | HP1084 | HP1085 | HP1088 | HP1091 | HP1092 |
| HP1093 | HP1094 | HP1095 | HP1096 | | | |

The expression of genes in this T1/T2 long loop is controlled by the following C1/C2 short loops.

A C1/C2 short loop on chromosome 1 whose identifier is 1241 controls the expression of the genes in this T1/T2 long loop. This C1/C2 short loop is expressed as a RNA single strand that is 3′UTR to the gene HP1535 and has the DNA sequence

TTTTACTCATAGGGTTTTTATAGTTCCTAGCGGAACTAAAGCATTCATCCC
AAACA

The match between the T1 sequence and the C1/C2 sequence is

TTTTACTCATAGGGTTTTTATAGTTCCTAGCGGAACTAAAGCA

The match between the T2 sequence and the C1/C2 sequence is

TAGCGGAACTAAAGCATTCATCCCAAACA

Example of a Single-celled Permanent Connectron—S. cervesiae

In this example the existence of the T1-T2 (5515-5533) long loop is controlled by a C1/C2 short loop (6102). The expression of this C1/C2 short loop is controlled only by the gene YNL339C.

```
                          6102 Chromosome 14
                          |
*------------------*------------------*
| Chromosome 12  |
5515  5533
-------------------------------------------------------
```

A double stranded DNA loop of length 6.466 kilo-bases on chromosome 12 is bounded on the left by a T1 sequence whose identifier is 5515. This T1 control element has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

This double stranded DNA loop is bounded on the right by a T2 control element whose identifier is 5533. This T2 control element has the DNA sequence

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

This long T1/T2 double stranded DNA loop modulates the expression of the following genes

YLR467W

The expression of genes in this T1/T2 long loop is controlled by the following C1/C2 short loops.

A C1/C2 short loop on chromosome 14 whose identifier is 6102 controls the expression of the genes in this T1/T2 long loop. This C1/C2 short loop is expressed as a RNA single strand that is 3′UTR to the gene YNL339C and has the DNA sequence

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTGAACATCCGGGTAAGAGACAACAGGGCT

The match between the T1 sequence and the C1/C2 sequence is

AGGAAATTGTTGTTACGAAAGTCAGTGATTATGTATTGTGTAGTATAGTAT
ATTGTAAGAAATTTTTTTTTCTAGGGAATATGCGTTTTGATGTAGTAGTAT
TTCACTGTTTTGATTTAGTGTTTGTTGCACGGCAGTAGCGAGAGACAAGTG
GGAAAGAGTAGGATAAAAAGACAATCTATAAAAAGTAAACATAAAATAA
AGGTAGTAAGTAGCTTTTGGTTG

The match between the T2 sequence and the C1/C2 sequence is

ATTATGTATTGTGTAGTATAGTATATTGTAAGAAATTTTTTTTTCTAGGGA
ATATGCGTTTTGATGTAGTAGTATTTCACTGTTTTGATTTAGTGTTTGTTGC
ACGGCAGTAGCGAGAGACAAGTGGGAAAGAGTAGGATAAAAAGACAATC
TATAAAAAGTAAACATAAAATAAAGGTAGTAAGTAGCTTTTGGTTGAACA
TCCGGGTAAGAGACAACAGGGCT

Example of a Multi-celled Permanent Connectron—C. elegans

In this example the existence of the T1-T2 (5515-5533) long loop is controlled by a C1/C2 short loop (6102). The expression of this C1/C2 short loop is controlled only by the gene YNL339C.

```
                          24442 Chromosome 5
                          |
*------------------*------------------*
| Chromosome 1  |
569  596
--------------------------------------------------------
```

A double stranded DNA loop of length 30.606 kilo-bases on chromosome 1 is bounded on the left by a T1 sequence whose identifier is 569. This T1 control element has the DNA sequence

AAATCGAGCCCGTAAATCGACACAAGCGCTACAGTAGTC

This double stranded DNA loop is bounded on the right by a T2 control element whose identifier is 596. This T2 control element has the DNA sequence

AGTGCTACAGTAGTCATTTAAAGAATTACTGTAGTTTTCGCT

The expression of genes in this T1/T2 long loop is controlled by the following C1/C2 short loops.

A C1/C2 short loop on chromosome 5 whose identifier is 24442 controls the expression of the genes in this T1/T2 long loop. This C1/C2 short loop is expressed as a RNA single strand that is 3′UTR to the gene F20D6.4 and has the DNA sequence

GAGCCCGTAAATCGACACAAGCGCTACAGTAGTCATTTAAAGAATTACTG
TAGTTTTC

The match between the T1 sequence and the C1/C2 sequence is

GAGCCCGTAAATCGACACAAGCGCTACAGTAGTC

The match between the T2 sequence and the C1/C2 sequence is

GCTACAGTAGTCATTTAAAGAATTACTGTAGTTTTC

7. Transient Connectrons Exist in Prokaryotes, Archea, Single-celled Eukaryotes and Multi-celled Eukaryotes.

A class of connectron relationships exist that permit one C1/C2 short loop to control the existence of one or more T1-T2 long loops such that this C1/C2 short loop is itself subject to expression control by another T1-T2 long loop which surrounds it. These connectron relationships are described as “transient”. Transient connectrons exist in prokaryotes, archea, single-celled eukaryotes and multi-celled eukaryotes.

Example of a Prokaryote Transient Connectron—E. coli

In this example the existence of the T1-T2 (3227-3329) long loop is controlled by the C1/C2(3225) short loop. The expression of this C1/C2 short loop is controlled by the existence of the T1-T2 (3216-3224) long loop. The existence of this T1-T2 long loop is itself determined by the expression of the C1/C2 (3223) short loop. The C1/C2 (3225) short loop is the transient connectron.

```
                          3223 Chromosome 1
                          |
*------------------*------------------*
| Chromosome 1  |
3216  3324
| 3225  |
                          3225 Chromosome 1
                          |
*------------------*-------------------*
| Chromosome 1  |
3227  3329
--------------------------------------------------------
```

A double stranded DNA loop of length 93.464 kilo-bases on chromosome 1 is bounded on the left by a T1 sequence whose identifier is 3216. This T1 control element has the DNA sequence

AGCGCAAGCGAAGCTCTTGATCGAAGCCCCGGTAAACGGCGGCCGTAACT
ATAACGGTCCTAAGGTAGCGAAATTCCTTGTCGGGTAAGTTCCGACCTGC
ACGAATGGCGTAATGATGGCCAGGCTGTCTCCACCCGAGACTCAGTGAAA
TTGAACTCGCTGTGAAGATGCAGTGTACCCGCGGCAAGACGGAAAGACCC
CGTGAACCTTTACTATAGCTTGACACTGAACATTGAGCCTTGATGTGTAGG
ATAGGTGGGAGGCTTTGAAGTGTGGACGCCAGTCTGCATGGAGCCGACCT
TGAAATACCACCCTTTAATGTTTGATGTTCTAACGT

This double stranded DNA loop is bounded on the right by a T2 control element whose identifier is 3324. This T2 control element has the DNA sequence

CCCGGTAAACGGCGGCCGTAACTATAACGGTCCTAAGGTAGCGAAATTCC
TTGTCGGGTAAGTTCCGACCTGCACGAATGGCGTAATGATGGCCAGGCTG
TCTCCACCCGAGACTCAGTGAAATTGAACTCGCTGTGAAGATGCAGTGTA
CCCGCGGCAAGACGGAAAGACCCCGTGAACCTTTACTATAGCTTGACACT
GAACATTGAGCCTTGATGTGTAGGATAGGTGGGAGGCTTTGAAGTGTGGA
CGCCAGTCTGCATGGAGCCGACCTTGAAATACCACCCTTTAATGTTTGATG
TTCTAACGTTGACCCGTAATCCGGGTTGCGGACAGT

This long T1/T2 double stranded DNA loop modulates the expression of the following genes

| | | | | | | | | |
|---|---|---|---|---|---|---|---|---|
| rrfC | aspT | trpT | yifA | yifE | yifB | ilvL | ilvG_1 | ilvM |
| ilvE | ilvD | ilvA | ilvY | ilvC | ppiC | b3776 | rep | gppA |
| rhlB | trxA | rhoL | rho | rfe | wzzE | wecB | rffH | wecD |
| wecE | wzxE | yifM_2 | wecG | yifK | argX | hisR | leuT | |
| proM | aslB | aslA | hemY | hemX | hemD | cyaA | cyaY | |
| b3808 | dapF | uvrD | b3814 | corA | yigF | yigG | rarD | yigI |
| pldA | recQ | yigJ | yigK | pldB | yigL | yigM | metR | metE |
| ysgA | udp | yigN | ubiE | yigP | b3836 | yigU | yigW_1 | rfaH |
| yigC | ubiB | fadA | fadB | pepQ | trkH | hemG | rrsA | ileT |
| rrlA | | | | | | | | |

This long T1/T2 double stranded DNA loop modulates the expression of the following C1/C2 short loops

A C1/C2 short loop on chromosome 1 whose identifier is 3225 controls the expression of the genes of one or more other T1/T2 long loops. This C1/C2 short loop is expressed as a RNA single strand that is 3′UTR to the gene rrlC and has the DNA sequence

AAACAGAATTTGCCTGGCGGCCGTAGCGCGGTGGTCCCACCTGACCCCAT
GCCGAACTCAGAAGTGAAACGCCGTAGCGCCGATGGTAGTGTGGGGTCTC
CCCATGCGAGAGTAGGGAACTGCCAGGCATCAAATTA

The expression of genes in this T1/T2 long loop is controlled by the following C1/C2 short loops.

A C1/C2 short loop on chromosome 1 whose identifier is 3323 controls the expression of the genes in this T1/T2 long loop. This C1/C2 short loop is expressed as a RNA single strand that is 3′UTR to the gene rrlA and has the DNA sequence

GCGAAGCTCTTGATCGAAGCCCCGGTAAACGGCGGCCGTAACTATAACGG
TCCTAAGGTAGCGAAATTCCTTGTCGGGTAAGTTCCGACCTGCACGAATG
GCGTAATGATGGCCAGGCTGTCTCCACCCGAGACTCAGTGAAATTGAACT
CGCTGTGAAGATGCAGTGTACCCGCGGCAAGACGGA...AACAGAATTTGC
CTGGCGGCAGTAGCGCGGTGGTCCCACCTGACCCCATGCCGAACTCAGAA
GTGAAACGCCGTAGCGCCGATGGTAGTGTGGGGTCTC

The match between the T1 sequence and the C1/C2 sequence is

GCGAAGCTCTTGATCGAAGCCCCGGTAAACGGCGGCCGTAACTATAACGG
TCCTAAGGTAGCGAAATTCCTTGTCGGGTAAGTTCCGACCTGCACGAATG
GCGTAATGATGGCCAGGCTGTCTCCACCCGAGACTCAGTGAAATTGAACT
CGCTGTGAAGATGCAGTGTACCCGCGGCAAGACGGAAAGACCCCGTGAA
CCTTTACTATAGCTTGACACTGAACATTGAGCCTTGATGTGTAGGATAGGT
GGGAGGCTTTGAAGTGTGGACGCCAGTCTGCATGGAGCCGACCTTGAAAT
ACCACCCTTTAATGTTTGATGTTCTAACGT

The match between the T2 sequence and the C1/C2 sequence is

CCCGGTAAACGGCGGCCGTAACTATAACGGTCCTAAGGTAGCGAAATTCC
TTGTCGGGTAAGTTCCGACCTGCACGAATGGCGTAATGATGGCCAGGCTG
TCTCCACCCGAGACTCAGTGAAATTGAACTCGCTGTGAAGATGCAGTGTA
CCCGCGGCAAGACGGAAAGACCCCGTGAACCTTTACTATAGCTTGACACT
GAACATTGAGCCTTGATGTGTAGGATAGGTGGGAGGCTTTGAAGTGTGGA
CGCCAGTCTGCATGGAGCCGACCTTGAAATACCACCCTTTAATGTTTGATG
TTCTAACGTTGACCCGTAATCCGGGTTGCGGACAGT

A double stranded DNA loop of length 93.749 kilo-bases on chromosome 1 is bounded on the left by a T1 sequence whose identifier is 3227. This T1 control element has the DNA sequence

AGCGCCGATGGTAGTGTGGGGTCTCCCCATGCGAGAGTAGGGAACTGCCA
GG

This double stranded DNA loop is bounded on the right by a T2 control element whose identifier is 3329. This T2 control element has the DNA sequence

CATGCGAGAGTAGGGAACTGCCAGGCATCAAATAAAACGAAAGGCTCAG
TCG

This long T1/T2 double stranded DNA loop modulates the expression of the following genes

| | | | | | | | | |
|---|---|---|---|---|---|---|---|---|
| aspT | trpT | yifA | yifE | yifB | ilvL | ilvG_1 | ilvM | ilvE |
| ilvD | ilvA | ilvY | ilvC | ppiC | b3776 | rep | gppA | rhlB |
| trxA | rhoL | rho | rfe | wzzE | wecB | rffH | wecD | wecE |
| wzxE | yifM_2 | wecG | yifK | argX | hisR | leuT | proM | |
| aslB | aslA | hemY | hemX | hemD | cyaA | cyaY | b3808 | |
| dapF | uvrD | b3814 | corA | yigF | yigG | rarD | yigI | pldA |
| recQ | yigJ | yigK | pldB | yigL | yigM | metR | metE | ysgA |
| udp | yigN | ubiE | yigP | b3836 | yigU | yigW_1 | rfaH | yigC |
| ubiB | fadA | fadB | pepQ | trkH | hemG | rrsA | ileT | rrlA |
| rrfA | | | | | | | | |

The expression of genes in this T1/T2 long loop is controlled by the following C1/C2 short loops.

A C1/C2 short loop on chromosome 1 whose identifier is 3225 controls the expression of the genes in this T1/T2 long loop. This C1/C2 short loop is expressed as a RNA single strand that is 3′UTR to the gene rrlC and has the DNA sequence

AAACAGAATTTGCCTGGCGGCCGTAGCGCGGTGGTCCCACCTGACCCCAT
GCCGAACTCAGAAGTGAAACGCCGTAGCGCCGATGGTAGTGTGGGGTCTC
CCCATGCGAGAGTAGGGAACTGCCAGGCATCAAATTA

The match between the T1 sequence and the C1/C2 sequence is

AGCGCCGATGGTAGTGTGGGGTCTCCCCATGCGAGAGTAGGGAACTGCCA
GG

The match between the T2 sequence and the C1/C2 sequence is

CATGCGAGAGTAGGGAACTGCCAGGCATCAAAT

Example of an Archea Transient Connectron—M. jannaschii

In this example the existence of the T1-T2 (1139-1159) long loop is controlled by the C1/C2 (533) short loop. The expression ofthis C1/C2 short loop is controlled by the existence of the T1-T2 (532-622) long loop. The existence of this T1-T2 long loop is itself determined by the expression of the C1/C2 (1629) short loop. The C1/C2 (533) short loop is the transient connectron.

```
                                1629 Chromosome 1
                                |
*------------------*------------------*
| Chromosome 1 |
532 622
| 533 |
                                533 Chromosome 1
                                |
*------------------*------------------*
| Chromosome 1 |
1139 1159
--------------------------------------------------------
```

A double stranded DNA loop of length 78.672 kilo-bases on chromosome 1 is bounded on the left by a T1 sequence whose identifier is 532. This T1 control element has the DNA sequence

ATATGTTTGAAATTTGAAAATAAGAGTATTTAG

This double stranded DNA loop is bounded on the right by a T2 control element whose identifier is 622. This T2 control element has the DNA sequence

TTGAAAATAAGAGCATTTAGAAGTTATTAATTAGTTCAAAGGATTTT

This long T1/T2 double stranded DNA loop modulates the expression of the following genes

| | | | | | | |
|---|---|---|---|---|---|---|
| MJ0486 | MJ0487 | MJ0488 | MJ0489 | MJ0490 | MJ0492 | MJ0493 |
| MJ0494 | MJ0495 | MJ0496 | MJ0497 | MJ0499 | MJ0500 | MJ0501 |
| MJ0502 | MJ0503 | MJ0504 | MJ0506 | MJ0507 | MJ0508 | MJ0509 |
| MJ0510 | MJ0511 | MJ0512 | MJ0513 | MJ0514 | MJ0514 | MJ0517 |
| MJ0519 | MJ0520 | MJ0521 | MJ0522 | MJ0523 | MJ0525 | MJ0526 |
| MJ0526 | MJ0529 | MJ0530 | MJ0531 | MJ0532 | MJ0534 | MJ0535 |
| MJ0536 | MJ0538 | MJ0539 | MJ0540 | MJ0541 | MJ0542 | MJ0543 |
| MJ0544 | MJ0545 | MJ0547 | MJ0548 | MJ0549 | MJ0550 | MJ0552 |
| MJ0553 | MJ0554 | MJ0555 | MJ0556 | MJ0557 | MJ0558 | MJ0559 |
| MJ0560 | MJ0561 | MJ0562 | MJ0563 | MJ0564 | MJ0565 | MJ0566 |
| MJ0568 | MJ0569 | MJ0570 | | | | |

This long T1/T2 double stranded DNA loop modulates the expression of the following C1/C2 short loops

A C1/C2 short loop on chromosome 1 whose identifier is 533 controls the expression of the genes of one or more other T1/T2 long loops. This C1/C2 short loop is expressed as a RNA single strand that is 3′UTR to the gene MJ0485 and has the DNA sequence

ATTTTTATTTAATTTCTAAGGGTTAGCTGGTTTGATTATTTAGAATATTT
GAGTTTATTGAATT

The expression of genes in this T1/T2 long loop is controlled by the following C1/C2 short loops.

A C1/C2 short loop on chromosome 1 whose identifier is 1629 controls the expression of the genes in this T1/T2 long loop. This C1/C2 short loop is expressed as a RNA single strand that is 3′UTR to the gene MJ1597 and has the DNA sequence

ATATGTTTGAAATTTGAAAATAAGAGTATTTAGAAGTTATTAATTAGTTCA
AAGGATTTTTATTTAATTTCTAAGGGTTTGCTGGTTTGATTATTTAGAATAT
TTGAGTTTATTGAATTATTCAGATTTTTAAAAATTA

The match between the T2 sequence and the C1/C2 sequence is

ATTTAGAAGTTATTAATTAGTTCAAAGGATTTT

A double stranded DNA loop of length 14.509 kilo-bases on chromosome 1 is bounded on the left by a T1 sequence whose identifier is 1139. This T1 control element has the DNA sequence

ATTTATTAATTAGTTCAAAGGATTTTTATTTAATTTCTAAGGGTTAGCTGG
TTTGATTGTTTAAAATATTTGAGTTTA

This double stranded DNA loop is bounded on the right by a T2 control element whose identifier is 1159. This T2 control element has the DNA sequence

ATTTAATTTCTAAGGGTTAGCTGGTTTGATTATTTAGAATATTTGAGTTTAT
TGAATTATTCAGATTTTTAAAAATTA

This long T1/T2 double stranded DNA loop modulates the expression of the following genes

| | | | | | | |
|---|---|---|---|---|---|---|
| MJ1096 | MJ1097 | tRNA-Arg-3 | MJ1098 | MJ1099 | MJ1100 | MJ1101 |
| MJ1102 | MJ1103 | MJ1104 | MJ1105 | MJ1106 | MJ1107 | MJ1108 |

The expression of genes in this T1/T2 long loop is controlled by the following C1/C2 short loops.

A C1/C2 short loop on chromosome 1 whose identifier is 533 controls the expression of the genes in this T1/T2 long loop. This C1/C2 short loop is expressed as a RNA single strand that is 3′UTR to the gene MJ0485 and has the DNA sequence

ATTTTTATTTAATTTCTAAGGGTTAGCTGGTTTGATTATTTAGAATATTTGA
GTTTATTGAATT

The match between the T1 sequence and the C1/C2 sequence is

ATTTTTATTTAATTTCTAAGGGTTAGCTGGTTTGATT

The match between the T2 sequence and the C1/C2 sequence is

ATTTAATTCTAAGGGTTAGCTGGTTTGATTATTTAGAATATTTGAGTTTAT
TGAATT

Example of a Single-celled Transient Connectron—S. cervesiae

In this example the existence of the T1-T2 (2840-2859) long loop is controlled by the C1/C2 (298) short loop. The expression of this C1/C2 short loop is controlled by the existence of the T1-T2 (293-320) long loop. The existence of this T1-T2 long loop is itself determined by the expression of the C1/C2 (86) short loop. The C1/C2 (298) short loop is the transient connectron.

```
                                86 Chromosome 1
                                |
*------------------*------------------*
| Chromosome 1 |
293 320
| 298 |
                                298 Chromosome 1
                                |
*------------------*------------------*
| Chromosome 7 |
2840 2859
--------------------------------------------------------
```

A double stranded DNA loop of length 38.470 kilo-bases on chromosome 2 is bounded on the left by a T1 sequence whose identifier is 293. This T1 control element has the DNA sequence

GAATTGTTGGAATAAAAATCCACTATCGTCTATCAACTAATAGTTATATTA
TCAATATATTATCATATACGGTGTTAAGATGATGACATAAGTTATGAGAA
GCTGTCATCGAAGTTAGAGGAAGCTGAAGTGCAAGGATTGATAATGTAAT
AGGATAATGAAACATATAAAACGGAATGAGGAATAATCGTAATATTAGT
ATGTAGAAATATAGATTCCATTTTGAGGATTCCTATATCCTTGAGGAGAAC
TTCTAGT

This double stranded DNA loop is bounded on the right by a T2 control element whose identifier is 320. This T2 control element has the DNA sequence

AATATTAGTATGTAGAAATATAGATTCCATTTTGAGGATTCCTATATCCTC
GAGGAGAACTTCTAGTATATTCTGTA

This long T1/T2 double stranded DNA loop modulates the expression of the following genes

| | | | | | |
|---|---|---|---|---|---|
| YBL005W-B | TS(AGA)B | YBL004W | YBL003C | YBL002W | YBL001C |
| YBR001C | YBR002C | YBR003W | YBR004C | YBR005W | YBR006W |
| YBR007C | YBR008C | YBR009C | YBR010W | YBR011C | YBR012C |

This long T1/T2 double stranded DNA loop modulates the expression of the following C1/C2 short loops

A C1/C2 short loop on chromosome 2 whose identifier is 298 controls the expression of the genes of one or more other T1/T2 long loops. This C1/C2 short loop is expressed as a RNA single strand that is 3′UTR to the gene YBL005W-B and has the DNA sequence

ATCTATTACATTATGGGTGGTATGTTGGAATAAAAATCCACTATCGTCTAT
CAACTAATAGTTATATTATCAATATATTATCATATACGGTGTTAAGATGAT
GACATAAGTTATGAGAAGCTGTCATCGAAGTTAGAGGAAGCTGAAGTGCA
AGGATTGATAATGTAATAGGATAATGAAACATATAAAACGGAATGAGGA
ATAATCGTAATATTAGTATGTAGAAATATAGATTCCATTTTGAGGATTCCT
ATATCCTTGAGGAGAACTTCTAGTATATTCTGTATACCTAATATTATAGCC
TTTATCAACAATGGAATCCCAACAATTATCTCAACATTC

The expression of genes in this T1/T2 long loop is controlled by the following C1/C2 short loops.

A C1/C2 short loop on chromosome 1 whose identifier is 86 controls the expression of the genes in this T1/T2 long loop. This C1/C2 short loop is expressed as a RNA single strand that is 3′UTR to the gene YAR009C and has the DNA sequence

ATCTATTACATTATGGGTGGTATGTTGGAATAGAAATCAACTATCATCTAC
TAACTAGTATTTACATTACTAGTATATTATCATATACGGTGTTAGAAGATG
ACGCAAATGATGAGAAATAGTCATCTAAATTAGTGGAAGCTGAAACGCA
AGGATTGATAATGTAATAGGATCAATGAATATAAACATATAAAACGGAAT
GAGGAATAATCGTAATATTAGTATGTAGAAATATAGATTCCATTTTGAGG
ATTCCTATATCCTCGAGGAGAACTTCTAGTATATTCTGTATACCTAATATT
ATAGCCTTTATCAACAATGGAATCCCAACAATTATCTCAACATTCACCCAT
TTCTCAGAA

The match between the T1 sequence and the C1/C2 sequence is

AAACATATAAAACGGAATGAGGAATAATCGTAATATTAGTATGTAGAAAT
ATAGATTCCATTTTGAGGATTCCTATATCCT

The match between the T2 sequence and the C1/C2 sequence is

AATATTAGTATGTAGAAATATAGATTCCATTTTGAGGATTCCTATATCCTC
GAGGAGAACTTCTAGTATATTCTGTA

A double stranded DNA loop of length 5.302 kilo-bases on chromosome 7 is bounded on the left by a T1 sequence whose identifier is 2840. This T1 control element has the DNA sequence

TCTGTTGGAATAAAAATCCACTATCGTCTATCAACTAATAGTTATATTATC
AATATATTATCATATACGGTGTTAAGATGATGACATAAGTTATGAGAAGC
TGTCATCGAAGTTAGAGGAAGCTGAAACGCAAGGATTGATAATGTAATAG
GATCAATGAATATAAACATATAAAACGGAATGAGGAATAATCGTAATATT
AGTATGTAGAAATATAGATTCCATTTTGAGGATTCCTATATCCTCGAGGAG
AACTTCTAGTATATTCTGTATACCTAAATTATAGCCTTTATCAACAATGGA
ATCCCAACAA

This double stranded DNA loop is bounded on the right by a T2 control element whose identifier is 2859. This T2 control element has the DNA sequence

CTATCAACTAATAGTTATATTATCAATATATTATCATATACGGTGTTAAGA
TGATGACATAAGTTATGAGAAGCTGTCATCGAAGTTAGAGGAAGCTGAAA
CGCAAGGATTGATAATGTAATAGGATCAATGAATATAAACATATAAAACG
GAATGAGGAATAATCGTAATATTAGTATGTAGAAATATAGATTCCATTTT
GAGGATTCCTATATCCTCGAGGAGAACTTCTAGTATATTCTGTATACCTAA
TATTATAGCCTTTATCAACAATGGAATCCCAACAATTATCTCAACATTCAC
ATATTTCTCAT

The expression of genes in this T1/T2 long loop is controlled by the following C1/C2 short loops.

A C1/C2 short loop on chromosome 2 whose identifier is 298 controls the expression of the genes in this T1/T2 long loop. This C1/C2 short loop is expressed as a RNA single strand that is 3′UTR to the gene YBL005W-B and has the DNA sequence

ATCTATTACATTATGGGTGGTATGTTGGAATAAAAATCCACTATCGTCTAT
CAACTAATAGTTATATTATCAATATATTATCATATACGGTGTTAAGATGAT
GACATAAGTTATGAGAAGCTGTCATCGAAGTTAGAGGAAGCTGAAGTGCA
AGGATTGATAATGTAATAGGATAATGAAACATATAAAACGGAATGAGGA
ATAATCGTAATATTAGTATGTAGAAATATAGATTCCATTTTGAGGATTCCT
ATATCCTTGAGGAGAACTTCTAGTATATTCTGTATACCTAATATTATAGCC
TTTATCAACAATGGAATCCCAACAATTATCTCAACATTC

The match between the T1 sequence and the C1/C2 sequence is

TGTTGGAATAAAAATCCACTATCGTCTATCAACTAATAGTTATATTATCAA
TATATTATCATATACGGTGTTAAGATGATGACATAAGTTATGAGAAGCTG
TCATCGAAGTTAGAGGAAGCTGAA

The match between the T2 sequence and the C1/C2 sequence is

CTATCAACTAATAGTTATATTATCAATATATTATCATATACGGTGTTAAGA
TGATGACATAAGTTATGAGAAGCTGTCATCGAAGTTAGAGGAAGCTGAA

Example of a Multi-celled Transient Connectron—C. elegans

In this example the existence of the T1-T2 (22072-22108) long loop is controlled by the C1/C2 (125) short loop. The expression of this C1/C2 short loop is controlled by the existence of the T1-T2 (110-129) long loop. The existence of this T1-T2 long loop is itself determined by the expression of the C1/C2 (16859) short loop. The C1/C2 (125) short loop is the transient connectron.

```
                              16859 Chromosome 4
                              |
*-----------------*-----------------*
| Chromosome 1 |
110 129
| 125 |
                              125 Chromosome 1
                              |
*-----------------*-----------------*
| Chromosome 5 |
22072 22108
--------------------------------------------------------
```

A double stranded DNA loop of length 18.855 kilo-bases on chromosome 1 is bounded on the left by a T1 sequence whose identifier is 110. This T1 control element has the DNA sequence

AGCTTAGGCTTAAGCTTAGGCTTAAGCTTAGGC

This double stranded DNA loop is bounded on the right by a T2 control element whose identifier is 129. This T2 control element has the DNA sequence

TTCTCCCGCATTTTTTGTAGATCTACGTAGATCAAACCGAAATGAGGCACT
TTCTGAATCCACGAGCTAGGCTTAAGCTTAGGCTTAAGCTTAGGCCTTTTC
TCAGGCTTAGGCTTAGGCTTA

This long T1/T2 double stranded DNA loop modulates the expression of the following genes

ZC123.3 ZC123.2

This long T1/T2 double stranded DNA loop modulates the expression of the following C1/C2 short loops

A C1/C2 short loop on chromosome 1 whose identifier is 125 controls the expression of the genes of one or more other T1/T2 long loops. This C1/C2 short loop is expressed as a RNA single strand that is 3′UTR to the gene ZC123.3 and has the DNA sequence

ACGCGCCGTAAATCTACCCCAGATATGGCCGAGCCAAAATGGCCTAGTTC
GGCAAACTCTTTCATTTCAATTTATGAGGGAAGCCAGAA

The expression of genes in this T1/T2 long loop is controlled by the following C1/C2 short loops.

A C1/C2 short loop on chromosome 4 whose identifier is 16859 controls the expression of the genes in this T1/T2 long loop. This C1/C2 short loop is expressed as a RNA single strand that is 3′UTR to the gene F58E2.7 and has the DNA sequence

CTTAGGCTTAAGCTTAGGCTTAAGCTTAGGCTTAAGCTTAGGCTTAAGCTT
AGGCTTAAGCTTAGGCTTAAGCTTAGGCTTAAGCTTAGGCTTAAGCTTAG
GCTTAAGCTTAGGCTTAAGCTTAGGCTTAAGCTTAGGCTTAAGCTTAGGCT
TAAGCTTAGACTTA

The match between the T1 sequence and the C1/C2 sequence is

AGCTTAGGCTTAAGCTTAGGCTTAAGCTTAGGC

The match between the T2 sequence and the C1/C2 sequence is

TAGGCTTAAGCTTAGGCTTAAGCTTAGGC

A double stranded DNA loop of length 51.031 kilo-bases on chromosome 5 is bounded on the left by a T1 sequence whose identifier is 22072. This T1 control element has the DNA sequence

CGCAACGCGCCGTAAATCTACCCCAGATATGGCCGAGCCAAAATGACCTA
GTTCGGC

This double stranded DNA loop is bounded on the right by a T2 control element whose identifier is 22108. This T2 control element has the DNA sequence

TGACAATCGCCTGCCGGACAACGCGTGGAAAAGTGTCGTGTACTCCACAC
GGACAAATACATTTAGTTTTACAACTAAAATCGAACCGCGACGCGACACG
CAACGCGACGTAAATCTACCCCAGATATGGCCGAGCCAAAATGGCCTAGT
TCGGCAAACTCTTCTATTTC

This long T1/T2 double stranded DNA loop modulates the expression of the following genes

F36H9.3 F36H9.4 F36H9.5 F36H9.2 F36H9.1 F36H9.6

The expression of genes in this T1/T2 long loop is controlled by the following C1/C2 short loops.

A C1/C2 short loop on chromosome 1 whose identifier is 125 controls the expression of the genes in this T1/T2 long loop. This C1/C2 short loop is expressed as a RNA single strand that is 3′UTR to the gene ZC123.3 and has the DNA sequence

ACGCGCCGTAAATCTACCCCAGATATGGCCGAGCCAAAATGGCCTAGTTC
GGCAAACTCTTTCATTTCAATTTATGAGGGAAGCCAGAA

The match between the T1 sequence and the C1/C2 sequence is

ACGCGCCGTAAATCTACCCCAGATATGGCCGAGCCAAAATG

The match between the T2 sequence and the C1/C2 sequence is

CGTAAATCTACCCCAGATATGGCCGAGCCAAAATGGCCTAGTTCGGCAAA
CTCTT

8. Self-limiting Connectrons Occur in Prokaryotes, Archea, Single-celled Eukaryotes and Multi-celled Eukaryotes

A class of connectron relationships exist that permit one C1/C2 short loop to control the existence of the T1-T2 long loop that surrounds it. These connectron relationships are described as “self-limiting”. Self-limiting connectrons exist in prokaryotes, archea, single-celled eukaryotes and multi-celled eukaryotes.

Example of a Prokaryotic Self-limiting Connectrons—E. coli

In this example the existence of the T1-T2 (1704-1718) long loop is controlled by two C1/C2 (1705 and 1713) short loops. The expression of these C1/C2 short loops is controlled by the existence of the T1-T2 (1704-1718) long loop. The existence of this T1-T2 long loop is itself determined by the expression of the two C1/C2 (1705 and 1713) short loops. The C1/C2 (1705 and 1713) short loops are the self-limiting connectrons.

```
                                1705 Chromosome 1
                                1713 Chromosome 1
                                |
*------------------*------------------*
| Chromosome 1 |
1704 1718
| 1705 1713 |
--------------------------------------------------------
```

A double stranded DNA loop of length 15.259 kilo-bases on chromosome 1 is bounded on the left by a T1 sequence whose identifier is 1704. This T1 control element has the DNA sequence

CGCCCCGTTCACACGATTCCTCTGTAGTTCAGTCGGTAGAACGGCGGACT
GTTAATCCGTATGTCACTGGT

This double stranded DNA loop is bounded on the right by a T2 control element whose identifier is 1718. This T2 control element has the DNA sequence

TTCAGTCGGTAGAACGGCGGACTGTTAATCCGTATGTCACTGGTTCGAGTC
CAGTCAGAGGAGCCAAATTC

This long T1/T2 double stranded DNA loop modulates the expression of the following genes

| | | | | | | | |
|---|---|---|---|---|---|---|---|
| asnT | b1978 | b1979 | b1980 | shiA | amn | b1983 | asnW |
| yeeO | asnU | | | | | | |

This long T1/T2 double stranded DNA loop modulates the expression of the following C1/C2 short loops

A C1/C2 short loop on chromosome 1 whose identifier is 1705 controls the expression of the genes of one or more other T1/T2 long loops. This C1/C2 short loop is expressed as a RNA single strand that is 3′UTR to the gene and has the DNA sequence

CGCCCCGTTCACACGATTCCTCTGTAGTTCAGTCGGTAGAACGGCGGACT
GTTAATCCGTATGTCACTGGTTCGAGTCCAGTCAGAGGAGCCAAATTC

A C1/C2 short loop on chromosome 1 whose identifier is 1713 controls the expression of the genes of one or more other T1/T2 long loops. This C1/C2 short loop is expressed as a RNA single strand that is 3′UTR to the gene asnW and has the DNA sequence

CACGATTCCTCTGTAGTTCAGTCGGTAGAACGGCGGACTGTTAATCCGTAT
GTCACTGGTTCGAGTCCAGTCAGAGGAGCCAAATT

The expression of genes in this T1/T2 long loop is controlled by the following C1/C2 short loops.

A C1/C2 short loop on chromosome 1 whose identifier is 1705 controls the expression of the genes in this T1/T2 long loop. This C1/C2 short loop is expressed as a RNA single strand that is 3′UTR to the gene and has the DNA sequence

CGCCCCGTTCACACGATTCCTCTGTAGTTCAGTCGGTAGAACGGCGGACT
GTTAATCCGTATGTCACTGGTTCGAGTCCAGTCAGAGGAGCCAAATTC

The match between the T1 sequence and the C1/C2 sequence is

CGCCCCGTTCACACGATTCCTCTGTAGTTCAGTCGGTAGAACGGCGGACT
GTTAATCCGTATGTCACTGGT

The match between the T2 sequence and the C1/C2 sequence is

TTCAGTCGGTAGAACGGCGGACTGTTAATCCGTATGTCACTGGTTCGAGTC
CAGTCAGAGGAGCCAAATTC

A C1/C2 short loop on chromosome 1 whose identifier is 1713 controls the expression of the genes in this T1/T2 long loop. This C1/C2 short loop is expressed as a RNA single strand that is 3′UTR to the gene asnW and has the DNA sequence

CACGATTCCTCTGTAGTTCAGTCGGTAGAACGGCGGACTGTTAATCCGTAT
GTCACTGGTTCGAGTCCAGTCAGAGGAGCCAAATT

The match between the T1 sequence and the C1/C2 sequence is

CACGATTCCTCTGTAGTTCAGTCGGTAGAACGGCGGACTGTTAATCCGTAT
GTCACTGGT

The match between the T2 sequence and the C1/C2 sequence is

TTCAGTCGGTAGAACGGCGGACTGTTAATCCGTATGTCACTGGTTCGAGTC
CAGTCAGAGGAGCCAAATT

Example of a Archea Self-limiting Connectrons—M. jannaschii

In this example the existence of the T1-T2 (1447-1471) long loop is controlled by two C1/C2 (1448 and 1470) short loops. The expression of these C1/C2 short loops is controlled by the existence of the T1-T2 (1447-1471) long loop. The existence of this T1-T2 long loop is itself determined by the expression of the two C1/C2 (1705 and 1713) short loops. The C1/C2 (1448 and 1470) short loops are the self-limiting connectrons.

```
                              1448 Chromosome 1
                              1470 Chromosome 1
                              |
*------------------*------------------*
| Chromosome 1 |
1447 1471
| 1448 1470 |
--------------------------------------------------------
```

A double stranded DNA loop of length 22.675 kilo-bases on chromosome 1 is bounded on the left by a T1 sequence whose identifier is 1447. This T1 control element has the DNA sequence

TTATAGAACATTATGAAGCTTTTTACTCAACTAACAACCGTATCGAATTTA
CCATTACTTGGAAATCTATTTAAAACCTCTTTAATCTTATGATA

This double stranded DNA loop is bounded on the right by a T2 control element whose identifier is 1471. This T2 control element has the DNA sequence

CAACTAACAACCGTATCGAATTTACCATTACTTGGAAATCTATTTAAAACC
TCTTTAATCTTGTGATAATAAATTCTAATCGATTCGTGACTTAT

This long T1/T2 double stranded DNA loop modulates the expression of the following genes

| | | | | | | |
|---|---|---|---|---|---|---|
| MJ1402 | MJ1403 | MJ1404 | MJ1405 | MJ1406 | MJ1407 | MJ1408 |
| MJ1409 | MJ1410 | MJ1411 | MJ1412 | MJ1413 | MJ1414 | MJ1415 |
| MJ1416 | MJ1417 | MJ1418 | MJ1419 | MJ1420 | | |

This long T1/T2 double stranded DNA loop modulates the expression of the following C1/C2 short loops

A C1/C2 short loop on chromosome 1 whose identifier is 1448 controls the expression of the genes of one or more other T1/T2 long loops. This C1/C2 short loop is expressed as a RNA single strand that is 3′UTR to the gene MJ1401 and has the DNA sequence

TTATAGAACATTATGAAGCTTTTTACTCAACTAACAACCGTATCGAATTTA
CCATTACTTGGAAATCTATTTAAAACCTCTTTAATCTTATGATAATAAATT
CTAATCGATTCGTGACTTAT

A C1/C2 short loop on chromosome 1 whose identifier is 1470 controls the expression of the genes of one or more other T1/T2 long loops. This C1/C2 short loop is expressed as a RNA single strand that is 3′UTR to the gene MJ1420 and has the DNA sequence

TTATAGAACATTATGAAGCTTTTTACTCAACTAACAACCGTATCGAATTTA
CCATTACTTGGAAATCTATTTAAAACCTCTTTAATCTTGTGATAATAAATT
CTAATCGATTCGTG

The expression of genes in this T1/T2 long loop is controlled by the following C1/C2 short loops.

A C1/C2 short loop on chromosome 1 whose identifier is 1470 controls the expression of the genes in this T1/T2 long loop. This C1/C2 short loop is expressed as a RNA single strand that is 3′UTR to the gene MJ1420 and has the DNA sequence

TTATAGAACATTATGAAGCTTTTTACTCAACTAACAACCGTATCGAATTTA
CCATTACTTGGAAATCTATTTAAAACCTCTTTAATCTTGTGATAATAAATT
CTAATCGATTCGTG

The match between the T1 sequence and the C1/C2 sequence is

TTATAGAACATTATGAAGCTTTTTACTCAACTAACAACCGTATCGAATTTA
CCATTACTTGGAAATCTATTTAAAACCTCTTTAATCTT

The match between the T2 sequence and the C1/C2 sequence is

CAACTAACAACCGTATCGAATTTACCATTACTTGGAAATCTATTTAAAACC
TCTTTAATCTTGTGATAATAAATTCTAATCGATTCGTG

A C1/C2 short loop on chromosome 1 whose identifier is 1448 controls the expression of the genes in this T1/T2 long loop. This C1/C2 short loop is expressed as a RNA single strand that is 3′UTR to the gene MJ1401 and has the DNA sequence

TTATAGAACATTATGAAGCTTTTTACTCAACTAACAACCGTATCGAATTTA
CCATTACTTGGAAATCTATTTAAAACCTCTTTAATCTTATGATAATAAATT
CTAATCGATTCGTGACTTAT

The match between the T1 sequence and the C1/C2 sequence is

TTATAGAACATTATGAAGCTTTTTACTCAACTAACAACCGTATCGAATTTA
CCATTACTTGGAAATCTATTTAAAACCTCTTTAATCTTATGATA

The match between the T2 sequence and the C1/C2 sequence is

CAACTAACAACCGTATCGAATTTACCATTACTTGGAAATCTATTTAAAACC
TCTTTAATCTT

Example of a Single-celled Self-limiting Connectron—S. cervesiae

In this example the existence of the T1-T2 (293-320) long loop is controlled by C1/C2 (298) short loop. The expression of this C1/C2 short loop is controlled by the existence of the T1-T2 (293-320) long loop. The existence of this T1-T2 long loop is itself determined by the expression of the C1/C2 (298) short loop. The C1/C2 (298) short loop is the self-limiting connectron.

```
                                298 Chromosome 2
                                |
*------------------*------------------*
| Chromosome 2 |
293 320
| 298 |
--------------------------------------------------------
```

A double stranded DNA loop of length 38.470 kilo-bases on chromosome 2 is bounded on the left by a T1 sequence whose identifier is 293. This T1 control element has the DNA sequence

GAATTGTTGGAATAAAAATCCACTATCGTCTATCAACTAATAGTTATATTA
TCAATATATTATCATATACGGTGTTAAGATGATGACATAAGTTATGAGAA
GCTGTCATCGAAGTTAGAGGAAGCTGAAGTGCAAGGATTGATAATGTAAT
AGGATAATGAAACATATAAAACGGAATGAGGAATAATCGTAATATTAGT
ATGTAGAAATATAGATTCCATTTTGAGGATTCCTATATCCTTGAGGAGAAC
TTCTAGT

This double stranded DNA loop is bounded on the right by a T2 control element whose identifier is 320. This T2 control element has the DNA sequence

AATATTAGTATGTAGAAATATAGATTCCATTTTGAGGATTCCTATATCCTC
GAGGAGAACTTCTAGTATATTCTGTA

This long T1/T2 double stranded DNA loop modulates the expression of the following genes

| | | | | | |
|---|---|---|---|---|---|
| YBL005W-B | TS(AGA)B | YBL004W | YBL003C | YBL002W | YBL001C |
| YBR001C | YBR002C | YBR003W | YBR004C | YBR005W | YBR006W |
| YBR007C | YBR008C | YBR009C | YBR010W | YBR011C | YBR012C |

This long T1/T2 double stranded DNA loop modulates the expression of the following C1/C2 short loops

A C1/C2 short loop on chromosome 2 whose identifier is 298 controls the expression of the genes of one or more other T1/T2 long loops. This C1/C2 short loop is expressed as a RNA single strand that is 3′UTR to the gene YBL005W-B and has the DNA sequence

ATCTATTACATTATGGGTGGTATGTTGGAATAAAAATCCACTATCGTCTAT
CAACTAATAGTTATATTATCAATATATTATCATATACGGTGTTAAGATGAT
GACATAAGTTATGAGAAGCTGTCATCGAAGTTAGAGGAAGCTGAAGTGCA
AGGATTGATAATGTAATAGGATAATGAAACATATAAAACGGAATGAGGA
ATAATCGTAATATTAGTATGTAGAAATATAGATTCCATTTTGAGGATTCCT
ATATCCTTGAGGAGAACTTCTAGTATATTCTGTATACCTAATATTATAGCC
TTTATCAACAATGGAATCCCAACAATTATCTCAACATTC

The expression of genes in this T1/T2 long loop is controlled by the following C1/C2 short loops.

A C1/C2 short loop on chromosome 2 whose identifier is 298 controls the expression of the genes in this T1/T2 long loop. This C1/C2 short loop is expressed as a RNA single strand that is 3′UTR to the gene YBL005W-B and has the DNA sequence

ATCTATTACATTATGGGTGGTATGTTGGAATAAAAATCCACTATCGTCTAT
CAACTAATAGTTATATTATCAATATATTATCATATACGGTGTTAAGATGAT
GACATAAGTTATGAGAAGCTGTCATCGAAGTTAGAGGAAGCTGAAGTGCA
AGGATTGATAATGTAATAGGATAATGAAACATATAAAACGGAATGAGGA
ATAATCGTAATATTAGTATGTAGAAATATAGATTCCATTTTGAGGATTCCT
ATATCCTTGAGGAGAACTTCTAGTATATTCTGTATACCTAATATTATAGCC
TTTATCAACAATGGAATCCCAACAATTATCTCAACATTC

The match between the T1 sequence and the C1/C2 sequence is

TGTTGGAATAAAAATCCACTATCGTCTATCAACTAATAGTTATATTATCAA
TATATTATCATATACGGTGTTAAGATGATGACATAAGTTATGAGAAGCTG
TCATCGAAGTTAGAGGAAGCTGAAGTGCAAGGATTGATAATGTAATAGGA
TAATGAAACATATAAAACGGAATGAGGAATAATCGTAATATTAGTATGTA
GAAATATAGATTCCATTTTGAGGATTCCTATATCCTTGAGGAGAACTTCTA
GT

The match between the T2 sequence and the C1/C2 sequence is

AATATTAGTATGTAGAAATATAGATTCCATTTTGAGGATTCCTATATCCT

Example of a Multi-celled Self-limiting Connectron—C. elegans

In this example the existence of the T1-T2 (293-320) long loop is controlled by C1/C2 (298) short loop. The expression of this C1/C2 short loop is controlled by the existence of the T1-T2 (293-320) long loop. The existence of this T1-T2 long loop is itself determined by the expression of the C1/C2 (298) short loop. The C1/C2 (298) short loop is the self-limiting connectron.

```
                                17155 Chromosome 4
                                |
*------------------*------------------*
| Chromosome 4 |
17154 17190
| 17155 |
--------------------------------------------------------
```

A double stranded DNA loop of length 89.919 kilo-bases on chromosome 4 is bounded on the left by a T1 sequence whose identifier is 17154. This T1 control element has the DNA sequence

AAATTTCCGGCAAATCGGCAAACTGGCAA

This double stranded DNA loop is bounded on the right by a T2 control element whose identifier is 17190. This T2 control element has the DNA sequence

AATTTGCCGATTTGCCGAATTTGTCGACA

This long T1/T2 double stranded DNA loop modulates the expression of the following genes

| | | | | | | |
|---|---|---|---|---|---|---|
| R08C7.11 | M01H9.2 | M01H9.3 | M01H9.4 | M01H9.1 | ZK180.1 | ZK180.2 |
| ZK180.3 | ZK180.4 | ZK180.5 | ZK180.6 | ZK185.3 | ZK185.2 | |

This long T1/T2 double stranded DNA loop modulates the expression of the following C1/C2 short loops

A C1/C2 short loop on chromosome 4 whose identifier is 17155 controls the expression of the genes of one or more other T1/T2 long loops. This C1/C2 short loop is expressed as a RNA single strand that is 3′UTR to the gene R08C7.1 and has the DNA sequence

AAATTTCCGGCAAATCGGCAAACTGGCAATTTGCCGATTTGCCGAATTTGT
CGACA

A C1/C2 short loop on chromosome 4 whose identifier is 17171 controls the expression of the genes of one or more other T1/T2 long loops. This C1/C2 short loop is expressed as a RNA single strand that is 3′UTR to the gene ZK180.2 and has the DNA sequence

TGGAAATTTCAGAATTTCAATTTTAATCGGCAAAATTGTACGCATCCTATG
AATTT

The expression of genes in this T1/T2 long loop is controlled by the following C1/C2 short loops.

A C1/C2 short loop on chromosome 4 whose identifier is 17155 controls the expression of the genes in this T1/T2 long loop. This C1/C2 short loop is expressed as a RNA single strand that is 3′UTR to the gene R08C7.1 and has the DNA sequence

AAATTTCCGGCAAATCGGCAAACTGGCAATTTGCCGATTTGCCGAATTTGT
CGACA

The match between the T1 sequence and the C1/C2 sequence is

AAATTTCCGGCAAATCGGCAAACTGGCAA

The match between the T2 sequence and the C1/C2 sequence is

AATTTGCCGATTTGCCGAATTTGTCGACA

9. Geneless Connectrons Exist in Single-celled and Multi-celled Eukaryotes

Normally T1-T2 long loops contain genes whose expression is regulated by the existence of the long loop. When a T1-T2 long loop does not contain any genes it is described as being “geneless”. The existence of the T1-T2 long loop is itself controlled by one or more C1/C2 short loops that may be on the same or different chromosomes. The geneless T1-T2 long loops must contain one or more C1/C2 short loops.

Example of a Single-celled Geneless Connectron—S. cervesiae

In this example the existence of the T1-T2 (1537-1559) long loop is controlled by three C1/C2 (3789, 5289 and 5753) short loops. The expression of 21 C1/C2 (1538 through 1558) short loops are controlled by the existence of the T1-T2 (1537-1559) long loop.

```
                                3789 Chromosome 9
                                5289 Chromosome 12
                                5753 Chromosome 13
                                |
*------------------*------------------*
| Chromosome 4 |
1537 1559
| 1538 through 1558 |
--------------------------------------------------------
```

A double stranded DNA loop of length 4.825 kilo-bases on chromosome 4 is bounded on the left by a T1 sequence whose identifier is 1537. This T1 control element has the DNA sequence

ATGAGATATATGTGGGTAATTAGATAATTGTTGGGATTCCATTGTTGATAA
AGGCTATAATATTAGGTATACAGAATATACTAGAAGTTCTCCTCGAGGAT
TTAGGAATCCATAAAAGGGAATCTGCAATTCTACACAATTCTATAAATAT
TATTATCATCGTTTTATATGTTAATATTCATTGATCCTATTACATTATCAAT
CCTTGCGTTTCAGCTTCCACTAATTTAGATGACTATTTCTCATCATTTGCGT
CATCTTCTAACACCGTATATGATAATATACTAGTAACGTAAATACTAGTTA
GTAGATGATAGTTGATTTTTATTCCAACATACCACCCATAATGTAATAGAT
CTAAT

This double stranded DNA loop is bounded on the right by a T2 control element whose identifier is 1559. This T2 control element has the DNA sequence

ATGAGATATATGTGGGTAATTAGATAATTGTTGGGATTCCATTGTTGATAA
AGGCTATAATATTAGGTATACAGAATATACTAGAAGTTCTCCTCGAGGAT
TTAGGAATCCATAAAAGGGAATCTGCAATTCTACACAATTCTATAAATAT
TATTATCATCGTTTTATATGTTAATATTCATTGATCCTATTACATTATCAAT
CCTTGCGTTTCAGCTTCCACTAATTTAGATGACTATTTCTCATCATTTGCGT
CATCTTCTAACACCGTATATGATAATATACTAGTAACGTAAATACTAGTTA
GTAGATGATAGTTGATTTTTATTCCAACATACCACCCATAATGTAATAGAT
CTAAT

There are no genes controlled by this T1/T2 loop.

This long T1/T2 double stranded DNA loop modulates the expression of the following C1/C2 short loops

A C1/C2 short loop on chromosome 4 whose identifier is 1538 controls the expression of the genes of one or more other T1/T2 long loops. This C1/C2 short loop has the DNA sequence

ATGAGATATATGTGGGTAATTAGATAATTGTTGGGATTCCATTGTTGATAA
AGGCTATAATATTAGGTATACAGAATATACTAGAAGTTCTCCTCGAGGAT
TTAGGAATCCATAAAAGGGAATCTGCAATTCTACACAATTCTATAAATAT
TATTATCATCGTTTTATATGTTAATATTCATTGATCCTATTACATTATCAAT
CCTTGCGTTTCAGCTTCCACTAATTTAGATGACTATTTCTCATCATTTGCGT
CATCTTCTAACACCGTATATGATAATATACTAGTAACGTAAATACTAGTTA
GTAGATGATAGTTGATTTTTATTCCAACATACCACCCATAATGTAATAGAT
CTAATGAATCCATTTGTTTGTTAATAGTTT

This T1-T2 loop also modulates the C1/C2 short loops numbered 1539 to 1557

A C1/C2 short loop on chromosome 4 whose identifier is 1558 controls the expression of the genes of one or more other T1/T2 long loops. This C1/C2 short loop has the DNA sequence

AGCTTCTCATAACTTATGTCATCATCTTAACACCGTATATGATAATATATT
GATAATATAACTTGTTGGAATAAAAATCAACTATCATCTACTAACTAGTAT
TTACGTTACTAGTATATTATCATATACGGTGTTAGAAGATGACGCAAATG
ATGAGAAATAGTCATCTAAATTAGTGGAAGCTGA...GTCTATCTGGCGAAT
ATAAATTTTTACGCTACACACGTCATCGACATCTAAATATGACAGTCGCTG
AACTGTTCTTAGATATCCATGCTATTTATGAAGAACAACAGGGATCGAGA
AACAG

The expression of genes in this T1/T2 long loop is controlled by the following C1/C2 short loops.

A C1/C2 short loop on chromosome 9 whose identifier is 3789 controls the expression of the genes in this T1/T2 long loop. This C1/C2 short loop is expressed as a RNA single strand that is 3′UTR to the gene YIL059C and has the DNA sequence

TTTATATGTTAATATTCATTGATCCTATTACATTATCAATCCTTGCGTTTCA
GCTTCCACTAATTTAGATGACTATTTCTCATCATTTGCGTCATCTTCTAACA
CCGTATATGATAATATACTAGTAACGTAAATACTAGTTAGTAGATGATAG
TTGATTTTTATTCCAACAGTAT

The match between the T1 sequence and the C1/C2 sequence is

TTTATATGTTAATATTCATTGATCCTATTACATTATCAATCCTTGCGTTTCA
GCTTCCACTAATTTAGATGACTATTTCTCATCATTTGCGTCATCTTCTAACA
CCGTATATGATAATATACTAGTAACGTAAATACTAGTTAGTAGATGATAG
TTGATTTTTATTCCAACA

The match between the T2 sequence and the C1/C2 sequence is

TTTATATGTTAATATTCATTGATCCTATTACATTATCAATCCTTGCGTTTCA
GCTTCCACTAATTTAGATGACTATTTCTCATCATTTGCGTCATCTTCTAACA
CCGTATATGATAATATACTAGTAACGTAAATACTAGTTAGTAGATGATAG
TTGATTTTTATTCCAACA

A C1/C2 short loop on chromosome 12 whose identifier is 5289 controls the expression of the genes in this T1/T2 long loop. This C1/C2 short loop is expressed as a RNA single strand that is 3′UTR to the gene YLR301W and has the DNA sequence

GGTGAATTTTGAGATAATTGTTGGGATTCCATTTTTAATAAGGCAATAATA
TTAGGTATGTAGAATATACTAGAAGTTCTCCTCGAGGATTTAGGAATCCAT
AAAAGGGAATCTGCAATTCTACACAATTCTATAAATATTATTATCATCGTT
TTATATGTTAATATTCATTGATCCTATTACATTATCAATCCTTGCGTTTCAG
CTTCCACTAATTTAGATGACTATTTCTCATCATTTGCGTCATCTTCTAACAC
CGTATATGATAATATACTAGTAACGTAAATACTAGTTAGTAGATGATAGT
TGATTTTTATTCCAACAC

The match between the T1 sequence and the C1/C2 sequence is

AGAATATACTAGAAGTTCTCCTCGAGGATTTAGGAATCCATAAAAGGGAA
TCTGCAATTCTACACAATTCTATAAATATTATTATCATCGTTTTATATGTTA
ATATTCATTGATCCTATTACATTATCAATCCTTGCGTTTCAGCTTCCACTAA
TTTAGATGACTATTTCTCATCATTTGCGTCATCTTCTAACACCGTATATGAT
AATATACTAGTAACGTAAATACTAGTTAGTAGATGATAGTTGATTTTTATT
CCAACA

The match between the T2 sequence and the C1/C2 sequence is

AGGATTTAGGAATCCATAAAAGGGAATCTGCAATTCTACACAATTCTATA
AATATTATTATCATCGTTTTATATGTTAATATTCATTGATCCTATTACATTA
TCAATCCTTGCGTTTCAGCTTCCACTAATTTAGATGACTATTTCTCATCATT
TGCGTCATCTTCTAACACCGTATATGATAATATACTAGTAACGTAAATACT
AGTTAGTAGATGATAGTTGATTTTTATTCCAACA

A C1/C2 short loop on chromosome 13 whose identifier is 5753 controls the expression of the genes in this T1/T2 long loop. This C1/C2 short loop is expressed as a RNA single strand that is 3′UTR to the gene YMR044W and has the DNA sequence

TTGAGAAATGGGGGAATGTTGAGATAATTGTTGGGATTCCATTGTTGATA
AAGGCTATAATATTAGGTATACAGAATATACTAGAAGTTCTCCTCAAGGA
TATAGGAATCCTCAAAATGGAATCTATATTTCTACATACTAATATTACGAT
TATTCCTCATTCCGTTTTATATGTTTCATTATCCTATTACATTATCAATCCT
TGCACTTCAGCTTCCTCTAACTTCGATGACAGCTTCTCATAACTTATGTCA
TCATCTTAACACCGTATATGATAATATATTGATAATATAACTATTAGTTGA
TAGACGATAGTGGATTTTTATTCCAACAT

The match between the T1 sequence and the C1/C2 sequence is

AGATAATTGTTGGGATTCCATTGTTGATAAAGGCTATAATATTAGGTATAC
AGAATATACTAGAAGTTCTCCTC

The match between the T2 sequence and the C1/C2 sequence is

TTGTTGGGATTCCATTGTTGATAAAGGCTATAATATTAGGTATACAGAATA
TACTAGAAGTTCTCCTCAAGGAT

Two Examples of Multi-celled Geneless Connectrons—C. elegans

In the first example the existence of the T1-T2 (2342-2344) long loop is controlled by the C1/C2 (24114) short loop. The expression of one C1/C2 (2343) short loop is controlled by the existence of the T1-T2 (2342-2344) long loop.

```
                                24114 Chromosome 5
                                |
*------------------*------------------*
| Chromosome 1 |
2342 2344
| 2343 |
--------------------------------------------------------
```

In the second example the existence of the T1-T2 (29221-29262) long loop is controlled by the C1/C2 (24114) short loop. The expression of one C1/C2 (2343) short loop is controlled by the existence of the T1-T2 (2342-2344) long loop.

```
                                4291 Chromosome 1
                                |
*------------------*------------------*
| Chromosome 5 |
29221 29262
| 29222 through 29261 |
--------------------------------------------------------
```

A double stranded DNA loop of length 67.059 kilo-bases on chromosome 1 is bounded on the left by a T1 sequence whose identifier is 2342. This T1 control element has the DNA sequence

TGAAAACTACAGTAATTCTTTAAATGACTACTGTAGC

This double stranded DNA loop is bounded on the right by a T2 control element whose identifier is 2344. This T2 control element has the DNA sequence

CTACTGTAGCGCTTGTGTCGATTTACGGGCTCGATTT

There are no genes controlled by this T1/T2 loop.

This long T1/T2 double stranded DNA loop modulates the expression of the following C1/C2 short loops

A C1/C2 short loop on chromosome 1 whose identifier is 2343 controls the expression of the genes of one or more other T1/T2 long loops. This C1/C2 short loop has the DNA sequence

TCGACACAAGCGCTACAGTAGCTATTTAAAGAATTACTGTAGTTTTCGCTA
CGAGATATTT

The expression of genes in this T1/T2 long loop is controlled by the following C1/C2 short loops.

A C1/C2 short loop on chromosome 5 whose identifier is 24114 controls the expression of the genes in this T1/T2 long loop. This C1/C2 short loop is expressed as a RNA single strand that is 3′UTR to the gene C13F10.5 and has the DNA sequence

GCGAAAACTACAGTAATTCTTTAAATGACTACTGTAGCGCTTGTGTCGATT
TACGGGCTCGATTTTCG

The match between the T1 sequence and the C1/C2 sequence is

GAAAACTACAGTAATTCTTTAAATGACTACTGTAGC

The match between the T2 sequence and the C1/C2 sequence is

CTACTGTAGCGCTTGTGTCGATTTACGGGCTCGATTT

A double stranded DNA loop of length 41.297 kilo-bases on chromosome 5 is bounded on the left by a T1 sequence whose identifier is 29221. This T1 control element has the DNA sequence

TTTAAATTTCCCGCCAAAAATTGACTGAAAATTTGGATTTTCTTTCCAAAA
ATTGACAGAAA

This double stranded DNA loop is bounded on the right by a T2 control element whose identifier is 29262. This T2 control element has the DNA sequence

TGAAAATTTGAATTTCCCGCCAAAAATTAAC

There are no genes controlled by this T1/T2 loop.

This long T1/T2 double stranded DNA loop modulates the expression of the following C1/C2 short loops

A C1/C2 short loop on chromosome 5 whose identifier is 29222 controls the expression of the genes of one or more other T1/T2 long loops. This C1/C2 short loop has the DNA sequence

AATTTCCCGCCAAAAATTGACTGAAAATTTGGATTTTCTTTCCAAAAATTG
ACAGAAA

This T1-T2 loop also modulates the C1/C2 short loops numbered 29223 to 29260

A C1/C2 short loop on chromosome 5 whose identifier is 29261 controls the expression of the genes of one or more other T1/T2 long loops. This C1/C2 short loop has the DNA sequence

AAAATTGACTGAAAATTTGAATTTCCAGCCAAAAATTGACTGAAAATTTG
AATT

The expression of genes in this T1/T2 long loop is controlled by the following C1/C2 short loops.

A C1/C2 short loop on chromosome 1 whose identifier is 4291 controls the expression of the genes in this T1/T2 long loop. This C1/C2 short loop is expressed as a RNA single strand that is 3′UTR to the gene Y43F8C.5 and has the DNA sequence

AAAATTAACTGAAAATTTGAATTTCCCGCCAAAAATTGACTGAAAATTTG
AATTTCCCGCCAAAAAAAATTGACTGAAAATTTGAATTTCCCGCCAAAAA
TTGACTGAAAATTTGAATTTCCCGCCAAAAATTAATTGAAAATTTGAATTT
CCCGCCAAAAATTAATTGAAACTTTGAATTTTCAA...ATTTCCCGCCAAAA
ATTAATTGAAACTTTGAATTTTCAAATTTCCCGCCAAAAATTGACTGAAAA
TTTGAATTTCCCGCCAAAAATTAATTGAAAATTTGAATTTTTGAATTTCCC
GCCAAAAATGACTGA

The match between the T1 sequence and the C1/C2 sequence is

TTTAAATTTCCCGCCAAAAATTGACTGAAAATTTG

The match between the T2 sequence and the C1/C2 sequence is

AAAAAAATTGACTGAAAATTTGAATTTCCCGCCAAAAATTGA

10. One Connectron Controls many Geneless Connectrons in Single-Celled and Multi-celled Eukaryotes

One C1/C2 short loop can control the existence of many geneless T1-T2 long loops.

Example of a Single-celled Geneless Connectron—S. cervesiae

In this example the existence of the three T1-T2 (1142-1156, 1242-1272 and 7102-7117) long loops is controlled by the C1/C2 (5289) short loop.

```
                            5289 Chromosome 12
                            |
*------------------*------------------*
| Chromosome 4 |
1142 1156
| 1143 through 1155 |
                            5289 Chromosome 12
                            |
*------------------*------------------*
| Chromosome 4 |
1243 1272
| 1244 through 1271 |
                            5289 Chromosome 12
                            |
*------------------*------------------*
| Chromosome 5 |
7102 7117
| 7103 through 7116 |
--------------------------------------------------------
```

A double stranded DNA loop of length 5.337 kilo-bases on chromosome 4 is bounded on the left by a T1 sequence whose identifier is 1142. This T1 control element has the DNA sequence

ATTTTGAGATAATTGTTGGGATTCCATTTTTAATAAGGCAATAATATTAGG
TATGTAGATATACTAGAAGTTCTCCTCGAGGATTTAGGAATCCATAAAAG
GGAATCTGCAATTCTACACAATTCTATAAATATTATTATCATCATTTTATA
TGTTAATATTCATTGATCCTATTACATTATCAATCCTTGCGTTTCAGCTTCC
ACTAATTTAGATGACTATTTCTCATCATTTGCGTCATCTTCTAACACCGTAT
ATGATAATATACTAGTAACGTAAATACTAGTTAGTAGATGATAGTTGATTT
TTATTCCAACA

This double stranded DNA loop is bounded on the right by a T2 control element whose identifier is 1156. This T2 control element has the DNA sequence

TTTTAATAAGGCAATAATATTAGGTATGTAGATATACTAGAAGTTCTCCTC
CAGGATTTAGGAATCCATAAAAGGGAATCTGCAATTCTACACAATTCTAT
AAATATTATTATCATCATTTTATATGTTAATATTCATTGATCCTATTACATT
ATCAATCCTTGCGTTTCAGCTTCCACTAATTTAGATGACTATTTCTCATCAT
TTGCGTCATCTTCTAACACCGTATATGATAATATACTAGTAACGTAAATAC
TAGTTAGTAGATGATAGTTGATTTTTATTCCAACAAGAA

There are no genes controlled by this T1/T2 loop.

This long T1/T2 double stranded DNA loop modulates the expression of the following C1/C2 short loops

A C1/C2 short loop on chromosome 4 whose identifier is 1143 controls the expression of the genes of one or more other T1/T2 long loops. This C1/C2 short loop has the DNA sequence

ATTTTGAGATAATTGTTGGGATTCCATTTTTAATAAGGCAATAATATTAGG
TATGTAGATATACTAGAAGTTCTCCTCGAGGATTTAGGAATCCATAAAAG
GGAATCTGCAATTCTACACAATTCTATAAATATTATTATCATCATTTTATA
TGTTAATATTCATTGATCCTATTACATTATCAAT...CTCTAAGTCTCATTGCC
TTTGTGCCAAAAAATCTGTTTCTAAATTTCTCTTCATTTGTAGACTTAATTA
TACTGATCGTTGATCTACTATCAGTAAGTAAGCCTTTAATAATTGGTTTCT
TGTTAAGTTCTTGCACAAGGTGACTGAGGTTATTCAATAGCGG

This T1-T2 loop also modulates the C1/C2 short loops numbered 1144 to 1154

A C1/C2 short loop on chromosome 4 whose identifier is 1155 controls the expression of the genes of one or more other T1/T2 long loops. This C1/C2 short loop has the DNA sequence

GAGGAGAACTTCTAGTATATCTACATACCTAATATTATTGCCTTATTAAAA
ATGGAATCCCAACAATTA

The expression of genes in this T1/T2 long loop is controlled by the following C1/C2 short loops.

A C1/C2 short loop on chromosome 12 whose identifier is 5289 controls the expression of the genes in this T1/T2 long loop. This C1/C2 short loop is expressed as a RNA single strand that is 3′UTR to the gene YLR301W and has the DNA sequence

GGTGAATTTTGAGATAATTGTTGGGATTCCATTTTTAATAAGGCAATAATA
TTAGGTATGTAGAATATACTAGAAGTTCTCCTCGAGGATTTAGGAATCCAT
AAAAGGGAATCTGCAATTCTACACAATTCTATAAATATTATTATCATCGTT
TTATATGTTAATATTCATTGATCCTATTACATTATCAATCCTTGCGTTTCAG
CTTCCACTAATTTAGATGACTATTTCTCATCATTTGCGTCATCTTCTAACAC
CGTATATGATAATATACTAGTACGTAAATACTAGTTAGTAGATGATAGTT
GATTTTTATTCCAACAC

The match between the T1 sequence and the C1/C2 sequence is

ATTTTGAGATAATTGTTGGGATTCCATTTTTAATAAGGCAATAATATTAGG
TATGTAGA

The match between the T2 sequence and the C1/C2 sequence is

TTTTAATAAGGCAATAATATTAGGTATGTAGA

A double stranded DNA loop of length 5.251 kilo-bases on chromosome 4 is bounded on the left by a T1 sequence whose identifier is 1243. This T1 control element has the DNA sequence

CGTGTTTTATCTCATGTTGTTCGTTTTGTTATTGAGATATATGTGGGTAATT
AGATAATTGTTGGGATTCCATTGTTGATAAAGGCTATAATATTAGGTATAC
AGAATATACTAGAAGTTCTCCTCGAGGATTTAGGAATCCATAAAAGGGAA
TCTGCAATTCTACACAATTCTATAAATATTATTATCATCGTTTTATATGTTA
ATATTCATTGATCCTATTACATTATCAATCCTTGCGTTTCAGCTTCCACTAA
TTTAGATGACTATTTCTCATCATTTGCGTCATCTTCTAACACCGTATATGAT
AATATACTAGTAACGTAAATACTAGTTAGTAGATGATAGTTGATTTTTATT
CCAACA

This double stranded DNA loop is bounded on the right by a T2 control element whose identifier is 1272. This T2 control element has the DNA sequence

TGAGATATATGTGGGTAATTAGATAATTGTTGGGATTCCATTGTTGATAAA
GGCTATAATATTAGGTATACAGAATATACTAGAAGTTCTCCTCGAGGATTT
AGGAATCCATAAAAGGGAATCTGCAATTCTACACAATTCTATAAATATTA
TTATCATCGTTTTATATGTTAATATTCATTGATC...TATACTAGTAACGTAA
ATACTAGTTAGTAGATGATAGTTGATTTTTATTCCAACAGTTATAAGGTTG
TTTCATATGTGTTTTATGAA

There are no genes controlled by this T1/T2 loop.

This long T1/T2 double stranded DNA loop modulates the expression of the following C1/C2 short loops

A C1/C2 short loop on chromosome 4 whose identifier is 1244 controls the expression of the genes of one or more other T1/T2 long loops. This C1/C2 short loop has the DNA sequence

TTTATCTCATGTTGTTCGTTTTGTTATTGAGATATATGTGGGTAATTAGATA
ATTGTTGGGATTCCATTGTTGATAAAGGCTATAATATTAGGTATACAGAAT
ATACTAGAAGTTCTCCTCGAGGATTTAGGAATCCATAAAAGGGAATCTGC
AATTCTACACAATTCTATAAATATTATTATCAT...GTCTCGATGTAGTATAC
GTATAAATTATTACCTGATACTTCATCTCTAAGTCTCATTGCCTTTGTGCCA
AAAAATCTGTTTCTAAATTTCTCTTCATTTGTAGACTTAATTATACTGATCG
TTGATCTACTATCAGTAAGT

This T1-T2 loop also modulates the C1/C2 short loops numbered 1245 to 1270

A C1/C2 short loop on chromosome 4 whose identifier is 1271 controls the expression of the genes of one or more other T1/T2 long loops. This C1/C2 short loop has the DNA sequence

TGTTGTATCTCAAAATGAGATATGTCAGTATGACAATACGTCATCCTAAAC
GTTCATAAAACACATATGAAACAACCTTATAACTGTTGGAATAAAAATCA
ACTATCATCTACTAACTAGTATTTACGTTACTAGTATATTATCATATACGG
TGTTAGAAGATGACGCAAATGATGAGAAATAGTC...CAACAATGGAATCC
CAACAATTATCTAATTACCCACATATATCTCATGGTAGCGCCTGTGCTTCG
GTTACTTCTAAGGAAGTCGACAGAAATCAAGATCCGTTAGACGTTTCAGC
TTCCAAAA

The expression of genes in this T1/T2 long loop is controlled by the following C1/C2 short loops.

A C1/C2 short loop on chromosome 12 whose identifier is 5289 controls the expression of the genes in this T1/T2 long loop. This C1/C2 short loop is expressed as a RNA single strand that is 3′UTR to the gene YLR301W and has the DNA sequence

GGTGAATTTTGAGATAATTGTTGGGATTCCATTTTTAATAAGGCAATAATA
TTAGGTATGTAGAATATACTAGAAGTTCTCCTCGAGGATTTAGGAATCCAT
AAAAGGGAATCTGCAATTCTACACAATTCTATAAATATTATTATCATCGTT
TTATATGTTAATATTCATTGATCCTATTACATTATCAATCCTTGCGTTTCAG
CTTCCACTAATTTAGATGACTATTTCTCATCATTTGCGTCATCTTCTAACAC
CGTATATGATAATATACTAGTAACGTAAATACTAGTTAGTAGATGATAGT
TGATTTTTATTCCAACAC

The match between the T1 sequence and the C1/C2 sequence is

AGAATATACTAGAAGTTCTCCTCGAGGATTTAGGAATCCATAAAAGGGAA
TCTGCAATTCTACACAATTCTATAAATATTATTATCATCGTTTTATATGTTA
ATATTCATTGATCCTATTACATTATCAATCCTTGCGTTTCAGCTTCCACTAA
TTTAGATGACTATTTCTCATCATTTGCGTCATCTTCTAACACCGTATATGAT
AATATACTAGTAACGTAAATACTAGTTAGTAGATGATAGTTGATTTTTATT
CCAACA

The match between the T2 sequence and the C1/C2 sequence is

AGAATATACTAGAAGTTCTCCTCGAGGATTTAGGAATCCATAAAAGGGAA
TCTGCAATTCTACACAATTCTATAAATATTATTATCATCGTTTTATATGTTA
ATATTCATTGATCCTATTACATTATCAATCCTTGCGTTTCAGCTTCCACTAA
TTTAGATGACTATTTCTCATCATTTGCGTCATCTTCTAACACCGTATATGAT
AATATACTAGTAACGTAAATACTAGTTAGTAGATGATAGTTGATTTTTATT
CCAACA

A double stranded DNA loop of length 5.296 kilo-bases on chromosome 15 is bounded on the left by a T1 sequence whose identifier is 7102. This T1 control element has the DNA sequence

CATGATTAATATGACCAATCGGCGTGTGTTTTTGAAAAGTGGGTGAATTTT
GAGATAATTGTTGGGATTCCATTTTTAATAAGGCAATAATATTAGGTATGT
AGAATGTACTAGAAGTTCTCCTCAAGGATTTAGGAATCCATGAAAGGGAA
TCTGCAATTCTACACAATTCTATAAATATTATTATCATCATTTTATATGTTA
ATATTCATTGATCCTATTACATTATCAATCCTTGCGTTTCAGCTTCCACTAA
TTTAGATGACTATTTCTCATCATTTGCGTCATCTTCTAACACCGTATATGAT
AATATACTAGTAACGTAAATACTAGTTAGTAGATGATAGTTGATTTTTATT
CCAACA

This double stranded DNA loop is bounded on the right by a T2 control element whose identifier is 7117. This T2 control element has the DNA sequence

TGAAAAGTGGGTGAATTTTGAGATAATTGTTGGGATTCCATTTTTAATAAG
GCAATAATATTAGGTATGTAGAATGTACTAGAAGTTCTCCTCAAGGATTT
AGGAATCCATGAAAGGGAATCTGCAATTCTACACAATTCTATAAATATTA
TTATCATCATTTTATATGTTAATATTCATTGATCCTATTACATTATCAATCC
TTGCGTTTCAGCTTCCACTAATTTAGATGACTATTTCTCATCATTTGCGTCA
TCTTCTAACACCGTATATGATAATATACTAGTAACGTAAATACTAGTTAGT
AGATGATAGTTGATTTTTATTCCAACAGTTTTATATACCTCTCTTATTTAGT
ATAAGAA

There are no genes controlled by this T1/T2 loop.

This long T1/T2 double stranded DNA loop modulates the expression of the following C1/C2 short loops

A C1/C2 short loop on chromosome 15 whose identifier is 7103 controls the expression of the genes of one or more other T1/T2 long loops. This C1/C2 short loop has the DNA sequence

AAGAACATTGCTGATGTGATGACAAAACCTCTTCCGATAAAAACATTTAA
ACTATTAACTAACAAATGGATTCATTAGATCTATTACATTATGGGTGGTAT
GTTGGAATAAAAATCAACTATCATCTACTAACTAGTATTTACGTTACTAGT
ATATTATCATATACGGTGTTAGAAGATGACGCAAATGATGAGAAATAGTC
ATCTAAATTAGTGGAAGCTGAAACGCAAGGATTGATAATGTAATAGGATC
AATGAATATTAACATATAAAATGATGATAATAATATTTATAGAATTGTGT
AGAATTGCAGATTCCCTTTCATGGATTCCTAAATCCTTGAGGAGAACTTCT
AGTA

This T1-T2 loop also modulates the C1/C2 short loops numbered 7104 to 7115

A C1/C2 short loop on chromosome 15 whose identifier is 7116 controls the expression of the genes of one or more other T1/T2 long loops. This C1/C2 short loop has the DNA sequence

CCATTCTGTGGAGGTGGTACTGAAGCAGGTTGAGGAGAGACATGATGATG
GTTCTCTGGAACAGCT

The expression of genes in this T1/T2 long loop is controlled by the following C1/C2 short loops.

A C1/C2 short loop on chromosome 12 whose identifier is 5289 controls the expression of the genes in this T1/T2 long loop. This C1/C2 short loop is expressed as a RNA single strand that is 3′UTR to the gene YLR301W and has the DNA sequence

GGTGAATTTTGAGATAATTGTTGGGATTCCATTTTTAATAAGGCAATAATA
TTAGGTATGTAGAATATACTAGAAGTTCTCCTCGAGGATTTAGGAATCCAT
AAAAGGGAATCTGCAATTCTACACAATTCTATAAATATTATTATCATCGTT
TTATATGTTAATATTCATTGATCCTATTACATTATCAATCCTTGCGTTTCAG
CTTCCACTAATTTAGATGACTATTTCTCATCATTTGCGTCATCTTCTAACAC
CGTATATGATAATATACTAGTAACGTAAATACTAGTTAGTAGATGATAGT
TGATTTTTATTCCAACAC

The match between the T1 sequence and the C1/C2 sequence is

GGTGAATTTTGAGATAATTGTTGGGATTCCATTTTTAATAAGGCAATAATA
TTAGGTATGTAGAAT

The match between the T2 sequence and the C1/C2 sequence is

GGTGAATTTTGAGATAATTGTTGGGATTCCATTTTTAATAAGGCAATAATA
TTAGGTATGTAGAAT

Example of a Multi-celled Geneless Connectron—C. elegans

In this example the existence of the three T1-T2 (1142-1156, 14840-15042 and 15365-15627) long loops is controlled by the C1/C2 (16760) short loop.

```
                                16760 Chromosome 4
                                |
*------------------*------------------*
| Chromosome 4 |
1142 1156
| 3103 through 3119 |
                                16760 Chromosome 4
                                |
*------------------*------------------*
| Chromosome 4 |
14840 15042
| 14841 through 15401 |
                                16760 Chromosome 4
                                |
*------------------*------------------*
| Chromosome 5 |
15365 15627
| 15366 through 15625 |
--------------------------------------------------------
```

A double stranded DNA loop of length 15.894 kilo-bases on chromosome 1 is bounded on the left by a T1 sequence whose identifier is 3101. This T1 control element has the DNA sequence

CAAATCGGCAAATTGCCGGAATTGAACATTTCC

This double stranded DNA loop is bounded on the right by a T2 control element whose identifier is 3120. This T2 control element has the DNA sequence

AAACGATTTTTCCGGCAAATCGGCAAATTGCCGGAATTGTAATTTCCGGC
AAAT

There are no genes controlled by this T1/T2 loop.

This long T1/T2 double stranded DNA loop modulates the expression of the following C1/C2 short loops

A C1/C2 short loop on chromosome 1 whose identifier is 3103 controls the expression of the genes of one or more other T1/T2 long loops. This C1/C2 short loop has the DNA sequence

TTAAAATTTCCGGCAAATCGGCAAATTGGCAGAAATGAAACTCACGGCAA
ATCGG

This T1-T2 loop also modulates the C1/C2 short loops numbered 3104 to 3118

A C1/C2 short loop on chromosome 1 whose identifier is 3119 controls the expression of the genes of one or more other T1/T2 long loops. This C1/C2 short loop has the DNA sequence

CCCGCATTTTTTGTAGATCAAACCGTAATGGGACGGCCTGGCAACACGTG
ATTTTCCAAAT

The expression of genes in this T1/T2 long loop is controlled by the following C1/C2 short loops.

A C1/C2 short loop on chromosome 4 whose identifier is 16760 controls the expression of the genes in this T1/T2 long loop. This C1/C2 short loop is expressed as a RNA single strand that is 3′UTR to the gene T23E1.2 and has the DNA sequence

GGCAAATTGCCGAAATTGAACATTTCCGGCAAATCGGCAAATTGCCGGAA
TTGAACATTTCCGGCAAATCGGCAAATTGCCGGAATTGAACATTTCCGGC
AAATCGGCAAATTGCCGGAATTGA

The match between the T1 sequence and the C1/C2 sequence is

CAAATCGGCAAATTGCCGGAATTGAACATTTCC

The match between the T2 sequence and the C1/C2 sequence is

TTTCCGGCAAATCGGCAAATTGCCGGAATTG

A double stranded DNA loop of length 86.977 kilo-bases on chromosome 3 is bounded on the left by a T1 sequence whose identifier is 14840. This T1 control element has the DNA sequence

AAAAATTTCCGGCAAGTCGGCAATTTTCCGAAAATGAAAATTTCCGGCAA
ATCGGCAAATTGCCGGAATTGAAAATTCCTGGCAAATCAGCAAATTTGCG
GCAAATCGGCAATTTGCCGAAAATGAAAATTTCCGGCAAAT

This double stranded DNA loop is bounded on the right by a T2 control element whose identifier is 15042. This T2 control element has the DNA sequence

CAAATCGGTAGGTAAATTGGCCAAACTTGAAAATTTCCGGCAAATCGGCA
AATTCCGCGAACTGAACATTTCCGGCAAATCGGCAAATTGCTCGAACT

There are no genes controlled by this T1/T2 loop.

This long T1/T2 double stranded DNA loop modulates the expression of the following C1/C2 short loops

A C1/C2 short loop on chromosome 3 whose identifier is 14841 controls the expression of the genes of one or more other T1/T2 long loops. This C1/C2 short loop has the DNA sequence

AAAAATTTCCGGCAAGTCGGCAATTTTCCGAAAATGAAAATTTCCGGGAA
ATCGGCAAATTGCCGGAATTGAAAATTCCTGGCAAATCAGCAAATTTGCG
GCAAATCGGCAATTTGCCGAAAATGAAAATTTCCGGCAAAT

This T1-T2 loop also modulates the C1/C2 short loops numbered 14842 to 15040

A C1/C2 short loop on chromosome 3 whose identifier is 15041 controls the expression of the genes of one or more other T1/T2 long loops. This C1/C2 short loop has the DNA sequence

CGGCAATTGCCGTTCGGCAATTTGCCAATTTGCCGGAAATTTTCAATTCCG
GCAA

The expression of genes in this T1/T2 long loop is controlled by the following C1/C2 short loops.

A C1/C2 short loop on chromosome 4 whose identifier is 16760 controls the expression of the genes in this T1/T2 long loop. This C1/C2 short loop is expressed as a RNA single strand that is 3′UTR to the gene T23E1.2 and has the DNA sequence

GGCAAATTGCCGAAATTGAACATTTCCGGCAAATCGGCAAATTGCCGGAA
TTGAACATTTCCGGCAAATCGGCAAATTGCCGGAATTGAACATTTCCGGC
AAATCGGCAAATTGCCGGAATTGA

The match between the T1 sequence and the C1/C2 sequence is

ATTTCCGGCAAATCGGCAAATTGCCGGAATTGAA

The match between the T2 sequence and the C1/C2 sequence is

TGAACATTTCCGGCAAATCGGCAAATTGC

A double stranded DNA loop of length 98.488 kilo-bases on chromosome 3 is bounded on the left by a T1 sequence whose identifier is 15365. This T1 control element has the DNA sequence

AAAATTTCCGGCAAATCGGCAATTTGCCAAAAATTGAAATTTCCGGCAAA
TCGGCAATTTGTCAAAAATGAAAATTTCCGGCAAATCGGCAAATTGCCGA
AAATGAAAATTTCCGGCAAATCGGCAAACTTCCGGAACTGAAAATTTCCG
GCAAATCGGCAATTTGCCATAAATGAACATTTCCGG...GGCGAAAATTAAA
ATTTCCGCCATATCGGCAATTTGCCAAAAAATTAAAATTTCCGGCAAATC
GGCAAATTGCCGGAATTCAAAATTTCCGGCAAACCGGCAAATTGCCGGAA
CTCAAAATTCCCGGCAAATCAGCAAATTGCCGGAATT

This double stranded DNA loop is bounded on the right by a T2 control element whose identifier is 15627. This T2 control element has the DNA sequence

TGGCAAACCGGCAAATTGCCGGAATTGAACATTTCCGGCAAATCGGCAAT
TTGCCGGAATTGAAATTT

There are no genes controlled by this T1/T2 loop.

This long T1/T2 double stranded DNA loop modulates the expression of the following C1/C2 short loops

A C1/C2 short loop on chromosome 3 whose identifier is 15366 controls the expression of the genes of one or more other T1/T2 long loops. This C1/C2 short loop has the DNA sequence

TGCCGATTTGCCGGAAATTTTCATTTTCGGCAATTTGCCGATTTGCCGGAA
ATTTTCATT

This T1-T2 loop also modulates the C1/C2 short loops numbered 15366 to 15624

A C1/C2 short loop on chromosome 3 whose identifier is 15625 controls the expression of the genes of one or more other T1/T2 long loops. This C1/C2 short loop has the DNA sequence

TCAAGCAAATTGTCAAATTCGCGGAACTAAACATTTCCGGCAAATCGGCA
AATT

The expression of genes in this T1/T2 long loop is controlled by the following C1/C2 short loops.

A C1/C2 short loop on chromosome 4 whose identifier is 16760 controls the expression of the genes in this T1/T2 long loop. This C1/C2 short loop is expressed as a RNA single strand that is 3′UTR to the gene T23E1.2 and has the DNA sequence

GGCAAATTGCCGAAATTGAACATTTCCGGCAAATCGGCAAATTGCCGGAA
TTGAACATTTCCGGCAAATCGGCAAATTGCCGGAATTGAACATTTCCGGC
AAATCGGCAAATTGCCGGAATTGA

The match between the T1 sequence and the C1/C2 sequence is

ATTTCCGGCAAATCGGCAAATTGCCGGAATT

The match between the T2 sequence and the C1/C2 sequence is

CGGCAAATTGCCGGAATTGAACATTTCCGGCAAATCGGCAA

What is claimed is:

1. A method of identifying DNA sequences that control the expression of different collections of genes in a genome comprising, detecting selected DNA sequences adjacent to some genes excluding exons and introns.

2. A method of identifying DNA sequences that control the expression of different collections of genes comprising, detecting, by computer, one or more pairs of non-adjacent DNA sequences to which are bound to two RNA sequences.

3. A method of identifying DNA sequences that control the expression of different collections of genes in a genome comprising detecting changes in connectron behavior in the genome.

4. A method of modifying the expression of different gene collections in a genome, comprising detecting changes in connectron behavior as a result of an exogenous stimulus.

5. A method of detecting where and when new genes are being integrated into a host genome comprising detecting the connectrons in said host genome.

6. A method of detecting the expression effect of different gene collections in a given body comprising detecting the back and forth flow of connectrons between the chromosomes thereof.

7. A method of modifying a given body comprising modifying the connectron organization therein.

8. A method of detecting connectron control and target sequences in a given genome comprising:
determining the base composition of said genome,
determining one or more sites of control sequence organization, and/or
determining one or more sites of target application.

9. A method of determining the response of a cell in any tissue to changes in the cell's environment and/or genetic composition comprising providing a complete genomic DNA sequence for the organism and determining the effect of changes in connectrons due to application of a given exogenous stimulus to the gnome.

10. In prokaryotes, archea, single-celled eukaryotes and multi-celled eukaryotes, the tetradic relationship T1=CL and T2=C2 where T1 and T2 are DNA sequences 20 or more bases in length, where the C1 sequence is adjacent to the C2 sequence, where the T1 and T2 sequences are on the same chromosome, and where the C1/C2 sequences are on the same chromosome as T1 and T2 or where the C1/C2 sequences are on a chromosome different from T1 and T2, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 40 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart, and
T2 sequence—any positive or negative strand DNA sequence of 20 bases or more that is on the same chromosome as the T1 sequence, the T2 or T1 sequences must be between about 1 kb and 105 kb apart.

11. In prokaryotes, archea, single-celled eukaryotes and multi-celled eukaryotes, the connectron relationship that permits many different C1/C2 short loops to control the existence of a T1-T2 long loop and wherein said C1/C2 short lops can be on the same chromosome or on different chromosomes from the T1 -T2 long loop, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 540 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart, and
T2 sequence—any positive or negative strand DNA sequence of 20 bases or more that is on the same chromosome as the T1 sequence, the T2 or T1 sequences must be between about 1 kb and 105 kb apart.

12. In prokaryotes, archea, single-celled eukaryotes and multi-celled eukaryotes, the connectron relationship that permits one C1/C2 short loop to control the existence of many T1-T2 long loops, the C1/C2 short loop can be on the same chromosome or on different chromosomes from the T1-T2 long loops, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 40 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart, and
T2 sequence—any positive or negative strand DNA sequence of 20 bases or more that is on the same chromosome as the T1 sequence, the T2 or T1 sequences must be between about 1 kb and 105 kb apart.

13. The connectron relationships between prokaryotes and their plasmids wherein said connectrons implement a control mechanism between the two genomes that makes it possible from them to form a symbiotic relationship, and in the case of D. radiodurans the relationship is not symmetric, and the D. radiodurans genome sends C1/C2 short loops to the MP1 plasmid, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 40 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart, and
T2 sequence—any positive or negative strand DNA sequence of 20 bases or more that is on the same chromosome as the T1 sequence, the T2 or T1 sequences must be between about 1 kb and 105 kb apart.

14. The connectron relationships that exist in plant and higher animals.

15. In prokaryotes, archea, single-celled eukaryotes and multi-celled eukaryotes, the connectron relationship that permits one C1/C2 short loop to control the existence of one or more T1-T2 long loops without being subject to any expression controls other than those of the gene to which the C1/C2 is 3′UTR, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 40 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart,
T2 sequence—any positive or negative strand DNA sequence of 20 bases or more that is on the same chromosome as the T1 sequence, the T2 or T1 sequences must be between about 1 kb and 105 kb apart, and
3′UTR—untranslated 3′ end of an mRNA is beyond the end of the last exon, a stop codon in the mRNA causes the ribosome to stop the translation of mRNA into protein.

16. In prokaryotes, archea, single-celled eukaryotes and multi-celled eukaryotes, the connectron relationship that permits one C1/C2 short loop to control the existence of one or more T1-T2 long loops such that this C1/C2 short loop is itself subject to expression control by another T1-T2 long loop which surrounds it, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 40 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart, and
T2 sequence—any positive or negative strand DNA sequence of 20 bases or more that is on the same chromosome as the T1 sequence, the T2 or T1 sequences must be between about 1 kb and 105 kb apart.

17. In prokaryotes, archea, single-celled eukaryotes and multi-celled eukaryotes, the connectron relationship that permits one C1/C2 short loop to control the existence of the T1-T2 long loop that surrounds it, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 50 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart, and
T2 sequence—any positive or negative strand DNA sequence of 20 bases or more that is on the same chromosome as the T1 sequence, the T2 or T1 sequences must be between about 1 kb and 105 kb apart.

18. The connectron relationship that do not have any genes within the T1 -T2 long loop, wherein:
T1 sequence is any positive or negative strand DNA sequence of 20 bases or more that is on the same chromosome as the T2 sequence, and
T2 sequence—any positive or negative strand DNA sequence of 20 bases or more that is on the same chromosome as the T1 sequence, and the T2 or T1 sequences must be between about 1 kb and 105 kb apart.

19. The geneless connectron relationship where one C1/C2 short loop controls the existence of many geneless T1-T2 long loops, wherein:
C1 sequence—any positive or negative strand DNA sequence of 20 bases or more, the C2 sequence must occur in the same chromosome as the C1 sequence,
C2 sequence—any positive or negative strand DNA sequence of 20 bases or more, the C1 sequence must occur in the same chromosome as the C2 sequence,
C1/C2—any positive or negative strand DNA sequence of 40 or more bases such that the C1 sequence is adjacent to the C2 sequence,
T1 sequence—any positive or negative strand DNA sequence of 20 bases or more that is on the same chromosome as the T2 sequence, the T1 and T2 sequences must be between about 1 kb and 105 kb apart, and
T2 sequence—any positive or negative strand DNA sequence of 20 bases or more that is on the same chromosome as the T1 sequence, the T2 or T1 sequences must be between about 1 kb and 105 kb apart.
